(19) United States
(12) Patent Application Publication
(10) Pub. No.: US 2022/0152117 A1
(43) Pub. Date: May 19, 2022

(54) METHODS OF PROMOTING CELLULAR MATURATION WITH AMPK ACTIVATORS

(71) Applicant: UNIVERSITY OF WASHINGTON, Seattle, WA (US)

(72) Inventors: Charles E. MURRY, SEATTLE, WA (US); Xiulan YANG, SEATTLE, WA (US)

(73) Assignee: UNIVERSITY OF WASHINGTON, Seattle, WA (US)

(21) Appl. No.: 17/440,266

(22) PCT Filed: Mar. 17, 2020
(86) PCT No.: PCT/US2020/023142

§ 371 Date: Sep. 17, 2021

Related U.S. Application Data
(60) Provisional application No. 62/820,003, filed on Mar. 18, 2019.

(51) Int. Cl. A61K 35/34; A61K 45/06; A61P 3/04; A61P 3/10; C12N 5/0793; C12N 5/077; C12N 9/12

(52) U.S. Cl. CPC A61K 35/34; A61K 45/06; A61P 3/04; A61P 3/10; C12N 2506/45; C12N 5/0657; C12N 9/12; C12Y 207/11031; C12N 2501/727; C12N 5/0619

(57) ABSTRACT

Described herein are methods and compositions related to promoting maturation of in vitro-differentiated cardiomyocytes and in vitro-differentiated neurons, and methods and compositions using the resulting cardiomyocytes and neurons.

### CROSS-REFERENCE TO RELATED APPLICATIONS

This application claims benefit under 35 U.S.C. § 119(e) of U.S. Provisional Application No. 62/820,003 filed Mar. 18, 2019, the contents of which are incorporated herein by reference in their entireties.

### GOVERNMENT SUPPORT

This invention was made with government support under Contract Nos. R01 HL084642, P01 HL094374, U01 HL100405, and P01 GM081619 awarded by the National Institute of Health. The government has certain rights in the invention.

### SEQUENCE LISTING

The instant application contains a Sequence Listing which has been submitted electronically in ASCII format and is hereby incorporated by reference in its entirety. Said ASCII copy, created on Feb. 18, 2020, is named 034186-094360WOPT_SL.txt and is 28,268 bytes in size.

### TECHNICAL FIELD

The technology described herein relates to methods of promoting maturation of in vitro-differentiated cardiomyocytes and neurons and uses thereof.

### BACKGROUND

Promoting the maturation of in vitro-differentiated cells is essential for their use in a broad range of applications such as cardiac regenerative therapies, disease modeling, and drug screening. Many stem cell therapies (e.g., stem cell engraftment therapies for cardiovascular or neuronal disease) have shown a lack of clinical efficacy due, at least in part, to the inability to produce mature in vitro-differentiated cells. Thus, new cellular targets for promoting maturation of in-vitro differentiated cells, such as cardiomyocytes and neurons, are needed to improve the evaluation of cellular toxicity in drug screening platforms, develop improved disease models, and improve engraftment into mammalian subjects.

### SUMMARY

In one aspect, described herein is a method of promoting maturation of in vitro-differentiated cardiomyocytes, the method comprising treating in vitro-differentiated cardiomyocytes with an activator of adenosine monophosphate-activated protein kinase (AMPK).

In one embodiment, the treatment is for at least two days, three days, four days, five days, six days, one week, or two weeks.

In another embodiment, the activator of AMPK comprises a small molecule, a polypeptide, a nucleic acid encoding a polypeptide or a vector encoding a polypeptide.

In another embodiment, the small molecule is 5-aminoimidizole-4-carboxamide riboside (AICAR) or a derivative thereof that activates AMPK.

In another embodiment, the derivative is 5-aminoimidazole-4-carboxamide-1-β-D-ribofuranosyl-5′-monophosphate (ZMP).

In another embodiment, the polypeptide comprises AMPK.

In another embodiment, the activator comprises a vector encoding an AMPK polypeptide.

In another embodiment, the AMPK polypeptide is a constitutively active polypeptide.

In another embodiment, the nucleic acid encoding the polypeptide or the vector that encodes the polypeptide permits inducible expression of the polypeptide.

In another embodiment, the vector is selected from the group consisting of: a lentiviral vector, an adenoviral vector, an adeno-associated virus vector (AAV), episomal vector, an EBNA1 vector, a minicircle vector, and a Sendai virus vector.

In another embodiment, the in vitro-differentiated cardiomyocytes are human.

In another embodiment, the in vitro-differentiated cardiomyocytes are differentiated from induced pluripotent stem cells (iPSCs) or from embryonic stem cells.

In another embodiment, the in vitro differentiated cardiomyocytes are derived from a subject having a cardiac disease or disorder.

In another embodiment, the cardiac disease or disorder is selected from the group consisting of: arrhythmogenic right ventricular dysplasia (ARVD), cardiomyopathy, cardiac arrhythmia, cardiomyopathy, long QT syndrome, catecholaminergic polymorphic ventricular tachycardia (CPVT), Barth syndrome, and Duchenne muscular dystrophy-related cardiac disease.

In another embodiment, treatment with an activator of AMPK promotes one or more of electrical maturity, metabolic maturity, and/or contractile maturity of in vitro-differentiated cardiomyocytes.

In another embodiment, electrical maturity is determined by one or more of the following markers as compared to a reference level: increased gene expression of an ion channel gene, increased sodium current density, increased inwardly-rectifying potassium channel current density, decreased action potential frequency, decreased calcium wave frequency, and decreased field potential frequency.

In another embodiment, metabolic maturity of in vitro-differentiated cardiomyocytes is determined by one or more of the following markers as compared to a reference level: increased activity of mitochondrial function, increased fatty acid metabolism, increased oxygen consumption rate (OCR), increased phosphorylated acetyl CoA carboxylase (ACC) levels or activity, increased level or activity of fatty acid binding protein (FABP), increased level or activity of pyruvate dehydrogenase kinase-4 (PDK4), increased mitochondrial respiratory capacity, increased mitochondrial volume, and increased levels of mitochondrial DNA.

In another embodiment, contractile maturity is determined by one or more of the following markers as compared to a reference level: decreased beat frequency, increased contractile force, increased level or activity of α-myosin heavy chain (α-MHC), increased level or activity of sarcomeres, decreased circularity index, increased level or activity of troponin, increased level or activity of titin N2b, increased cell area, and increased aspect ratio.

In another embodiment, the method further comprises contacting the in vitro-differentiated cardiomyocytes with a nanopatterned substrate.

In another aspect, described herein is a method of transplanting in vitro-differentiated cardiomyocytes in a subject, the method comprises: (a) contacting in vitro-differentiated cardiomyocytes with an activator of AMPK; and (b) transplanting said in vitro-differentiated cardiomyocytes into the subject.

In one embodiment, the method further comprises administering metformin to the subject.

In another embodiment, the metformin modulates the electrical maturity, metabolic maturity, and/or contractile maturity of in vitro-differentiated cardiomyocytes.

In another embodiment, the metformin enhances engraftment of the in vitro-differentiated cardiomyocytes.

In another aspect, described herein is a method of promoting maturation of in vitro-differentiated neurons, the method comprising contacting in vitro-differentiated neurons with an activator of adenosine monophosphate-activated protein kinase (AMPK).

In one embodiment, the activator of AMPK comprises a small molecule, a polypeptide, a nucleic acid encoding a polypeptide or a vector encoding a polypeptide.

In another embodiment, the small molecule is 5-aminoimidizole-4-carboxamide riboside (AICAR) or a derivative thereof that activates AMPK.

In another embodiment, the derivative is 5-aminoimidazole-4-carboxamide-1-β-D-ribofuranosyl-5′-monophosphate (ZMP).

In another embodiment, the polypeptide comprises AMPK.

In another embodiment, the activator comprises a vector encoding an AMPK polypeptide.

In another embodiment, the AMPK polypeptide is a constitutively active polypeptide.

In another embodiment, the nucleic acid encoding the polypeptide or the vector that encodes the polypeptide permits inducible expression of the polypeptide.

In another embodiment, the vector is selected from the group consisting of: a lentiviral vector, an adenoviral vector, an adeno-associated virus vector (AAV), episomal vector, an EBNA1 vector, a minicircle vector, and a Sendai virus vector.

In another embodiment, the in vitro differentiated neurons are human.

In another embodiment, the in vitro-differentiated neurons are differentiated from induced pluripotent stem cells (iPSCs) or from embryonic stem cells.

In another embodiment, the in vitro-differentiated neurons are derived from a subject having a neurological disease or disorder.

In another embodiment, the neurological disease or disorder is selected from the group consisting of: Alzheimer's disease; Parkinson's disease; Down syndrome; dementia; multiple sclerosis; and amyotrophic lateral sclerosis (ALS).

In another embodiment, treatment with an activator of AMPK promotes a reduction in the level or activity of amyloid beta (Aβ) or phosphorylated Tau protein.

In another embodiment, treatment with an activator of AMPK promotes electrical maturity or metabolic maturity of in vitro-differentiated neurons.

In another embodiment, treatment with an activator of AMPK promotes maturity of in vitro-differentiated neurons as compared to a reference level, in one or more of the following markers of maturity: increased levels or activity of PPARα, increased levels or activity of TFAM, increased levels or activity of PDK4, increased levels or activity of NeuN, reduced levels or activity of amyloid beta (Aβ), reduced levels or activity of phosphorylated Tau protein, increased activity of mitochondrial function, increased fatty acid metabolism, and increased levels of mitochondrial DNA.

In another embodiment, the Aβ is Aβ₁₋₄₂.

In another aspect, described herein is a method of evaluating toxicity of an agent, the method comprising contacting in vitro-differentiated cardiomyocytes or neurons prepared by the methods described herein with an agent.

In one embodiment, the method further comprises detecting at least one phenotypic characteristic of the cardiomyocytes or neurons.

In another embodiment, the agent is selected from the group consisting of a small molecule, an antibody, a peptide, a genome editing system, and a nucleic acid.

In another embodiment, toxicity of an agent is indicated by the agent's effect on one or more of: cell viability, cell size, a biopotential or electrical property, mitochondrial function, gene expression, beat rate, and contractile function.

In another aspect, described herein is a composition comprising in vitro-differentiated cardiomyocytes made by contacting in vitro-differentiated cardiomyocytes with an activator of adenosine monophosphate-activated protein kinase (AMPK), wherein the cardiomyocytes have a more mature phenotype as compared with in vitro-differentiated cardiomyocytes that were not contacted with an activator of adenosine monophosphate-activated protein kinase (AMPK).

In another aspect, described herein is a composition comprising in vitro-differentiated neurons made by contacting in vitro-differentiated neurons with an activator of adenosine monophosphate-activated protein kinase (AMPK), wherein the neurons have a more mature phenotype as compared with in vitro-differentiated neurons that were not contacted with an activator of adenosine monophosphate-activated protein kinase (AMPK).

In another aspect, described herein is an activator of AMPK for use in promoting the maturation of in vitro-differentiated cardiomyocytes.

In another aspect, described herein is an activator of AMPK for use in promoting the maturation of in vitro-differentiated neurons.

In another aspect, described herein is a composition comprising in vitro-differentiated cardiomyocytes and an activator of AMPK for use in the treatment of a cardiac disease or disorder.

In another aspect, described herein is a composition comprising in vitro-differentiated neurons and an activator of AMPK for use in the treatment of a neurological disease or disorder.

In another aspect, described herein is the use of a composition comprising in vitro-differentiated cardiomyocytes that have been treated with an activator of AMPK for treatment of a cardiac disease or disorder.

In another aspect, described herein is the use of a composition comprising in vitro-differentiated neurons that have been treated with an activator of AMPK for treatment of a neurological disease or disorder.

In another aspect, described herein is a composition comprising in vitro-differentiated cardiomyocytes that have been treated or contacted with an activator of AMPK for use in transplant to cardiac tissue of a subject in need thereof.

In another aspect, described herein is a composition comprising in vitro-differentiated neurons that have been treated or contacted with an activator of AMPK for use in transplant to neuronal tissue of a subject in need thereof.

### BRIEF DESCRIPTION OF THE DRAWINGS

FIG. 1A-1D shows AMPK activation enhances fatty acid oxidation capacity of hPSC-CMs partly by phosphorylating ACC. FIG. 1A shows representative traces from palmitate XF96 extracellular flux assay. Note the marked increase in palmitate oxidation with AICAR treatment (red line); FIG. 1B shows statistical analysis of OCR increase induced by palmitate-albumin for control and AICAR-treated hPSC-CMs; FIG. 1C shows AICAR treatment led to robust ACC phosphorylation; FIG. 1D shows AMPK activation resulted in increased FABP and PDK4 expression levels. Gene expression is shown normalized first to HPRT mRNA levels and then normalized to control cells. #P<0.001, *P<0.05 versus control hPSC-CMs.

FIG. 2A-2E shows the effect of AMPK activation on mitochondrial function and biogenesis. FIG. 2A shows representative traces for control and AICAR treated hiPSC-CMs responding to the ATP synthase inhibitor oligomycin, the respiratory uncoupler FCCP, and the Complex I and Complex III inhibitors rotenone and antimycin A. FIG. 2B shows statistical analysis of the differences in mitochondrial maximum respiratory capacity. FIG. 2C shows he mtDNA to nDNA ratio after AICAR treatment relative to control hiPSC-CMs. FIG. 2D demonstrates relative mitochondrial volume determined using electron microscopy images with P<0.0001 between groups. FIG. 2E shows AMPK activation resulted in increased ERRα, PPARα, PGC-1α, and TFAM expression. Gene expression is shown normalized first to HPRT mRNA levels and then normalized to control cells. *P<0.05, #P<0.01 vs. control hPSC-CMs.

FIG. 3A-3I shows the effects of AMPK activation on hPSC-CM morphology and cardiac gene expression. Representative control (FIG. 3A) and AICAR-treated (FIG. 3B) cells were stained with α-actinin, F-actin, and Hoechst 33342. Scale bar: 20 μm. Compared to control hPSC-CMs, AICAR-treated cardiomyocytes exhibited significant changes in cell area (FIG. 3C), circularity index (FIG. 3D). AICAR treatment led to an increased α-MHC, KCNJ2, and SCN5a expression (FIG. 3E). AICAR treatment led to a significant increase in titin N2B (FIG. 3F) and cTnI (FIG. 3H) expression levels. As a result, the ratios of N2BA/N2B (FIG. 3G) and ssTnI/cTnI (FIG. 3I) were significantly downregulated. Gene expression is shown normalized first to HPRT mRNA levels and then normalized to control cells. *P<0.05, #P<0.001 vs control hPSC-CMs.

FIG. 4A-4F shows AMPK activation enhances contractility, passive tension, and reduces automaticity. FIG. 4A shows representative force traces generated by control and AICAR-treated hPSC-CMs. Total force per beat (FIG. 4B); average twitch force per post; and passive tension (FIG. 4D) were increased by AICAR treatment. FIG. 4E shows beating frequency was reduced with AICAR treatment. FIG. 4F shows cell area on the microposts was upregulated by AICAR treatment. *P<0.05, #P<0.001 versus control hPSC-CMs.

FIG. 5 shows 1 mM AICAR treatment activates multiple intracellular signal pathways in hPSC-CMs. The level of AMPK, ACC, Akt, ERK, and p38-MAPK phosphorylation were detected at the indicated time points after 1 mM AICAR treatment.

FIG. 6A-6E shows the expression of exogenous AMPKα1-CA and α2-CA in hPSC-CMs. FIG. 6A shows Western blot analysis of phospho-ACC and phospho-AMPK 72 hours after transfection with Ad-GFP or Ad-(α1+α2)-CA-AMPK. FIG. 6B shows XF96 palmitate assay after one week of Ad-GFP or Ad-(α1+α2)-CA-AMPK transduction. FIG. 6C shows Statistical analysis of OCR increase induced by palmitate-albumin for Ad-GFP or Ad-(α1+α2)-CA-AMPK-transduced hPSC-CMs. FIG. 6D shows representative traces for Ad-GFP or Ad-(α1+α2)-CA-AMPK-transduced hPSC-CMs responding to the ATP synthase inhibitor oligomycin, the respiratory uncoupler FCCP, and the Complex I and Complex III inhibitors rotenone and antimycin A in XF 96 assay. FIG. 6E shows statistical analysis of the differences in mitochondrial maximum respiratory capacity.

FIG. 7 shows induction of pathological features of ARVD/C through AMPK activation. Both AICAR and AICAR plus PPAR-γ agonists induced significant cell death in the cardiomyocytes-derived from a mutant PKP2-iPSCs than the cardiomyocytes from a normal iPSCs. The cells were stained with α-actinin (green), TUNEL (red), and DAPI (blue).

FIG. 8A-8D shows AMPK activation increases neuronal maturation and reduces secreted Aβ peptides from FAD patient cells. maturation. FIG. 8A shows quantitative PCR of the mtDNA:nDNA ratio in hiPSC-derived neurons after AICAR treatment. FIG. 8B shows AMPK activation resulted in increased PPARα, TFAM, PDK4, and NeuN expression. Gene expression is shown normalized first to HPRT mRNA levels and then normalized to control cells. *P<0.05, #P<0.01 vs. control hiPSC-derived neurons. FIG. 8C shows Treatment of neurons derived from an FAD patient cell line harboring a duplication of the amyloid precursor protein (APP gene) with AICAR significantly reduces secreted Aβ₁₋₄₀ peptides and (FIG. 8D) the more pathogenic Aβ₁₋₄₂ peptides.

FIG. 9A-9D shows expression of hepatic and mitochondrial-related genes in hiPS-HLCs. FIG. 9A shows a schematic diagram of differentiation of iPSCs to HLCs. FIG. 9B shows ALB and AFP expression analysis during the maturation step after addition of AICAR (1 mM) for 1 to 5 days between day 15 and 20 of differentiation, showed longer treatment with AICAR will affect hepatic maturation adversely by downregulating ALB while two-days treatment enhanced ALB and diminished AFP. Further analysis of mature hepatic specific genes expression of the two-days treated cells; days 15-17, with AICAR (1 mM) revealed significantly reduced AFP and HNF4α expression in the treated group (*p<0.05) while the treatment had no impact in other genes. (C). Mitochondrial related gene expression after one-week treatment with AICAR (untreated-1 w & 1 mM-1 w) showed non-significant reduction in most of the genes in treated and untreated cells in comparison to day 20, except for PDK4 which significantly increased with 1 mM AICAR (D) (*p<0.05).

### DETAILED DESCRIPTION

The compositions and methods described herein are related, in part, to a method of promoting maturation of in vitro-differentiated cardiomyocytes (hPSC-CMs) and neurons. Promoting the maturation of hPSC-CMs and neurons is essential for their use in a broad range of applications such as regenerative therapies, disease modeling, and drug screening.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein the term “human stem cell” refers to a human cell that can self-renew and differentiate to at least one cell type. The term “human stem cell” encompasses human stem cell lines, human-derived iPS cells, human embryonic stem cells, human pluripotent cells, human multipotent stem cells, amniotic stem cells, placental stem cells, or human adult stem cells.

As used herein, “in vitro-differentiated cardiomyocytes” refers to cardiomyocytes that are generated in culture, typically via step-wise differentiation from a precursor cell such as a human embryonic stem cell, an induced pluripotent stem cell, an early mesoderm cell, a lateral plate mesoderm cell or a cardiac progenitor cell.

The term “differentiate”, or “differentiating” is a relative term that indicates a “differentiated cell” is a cell that has progressed further down the developmental pathway than its precursor cell. Thus in some embodiments, a stem cell as the term is defined herein, can differentiate to lineage-restricted precursor cells (e.g. a human cardiac progenitor cell or mid-primitive streak cardiogenic mesoderm progenitor cell), which in turn can differentiate into other types of precursor cells further down the pathway (such as a tissue specific precursor, such as a cardiomyocyte or neuronal precursor), and then to an end-stage differentiated cell, which plays a characteristic role in a certain tissue type, and may or may not retain the capacity to proliferate further. Methods for in vitro differentiation of stem cells to cardiomyocytes and/or to neurons are known in the art and described herein.

As used herein, the terms, “maturation” or “mature phenotype” when applied to cardiomyocytes or neurons refers to the phenotype of a cell that comprises a phenotype similar to adult cardiomyocytes or neurons and does not comprise at least one feature of a fetal cardiomyocyte or neuron. In some embodiments, markers which indicate increased maturity of an in vitro-differentiated cell include, but are not limited to, electrical maturity, metabolic maturity, genetic marker maturity, and contractile maturity.

As used herein, “electrical maturity,” refers to a the enhance electrical properties or functional phenotype of a cell as described herein. Electrical maturity can be determined by one or more of the following markers compared to a reference level selected from the group consisting of: increased gene expression of ion channel genes, increased sodium current density, increased inwardly-rectifying potassium channel current density, decreased action potential frequency, decreased calcium wave frequency, and decreased field potential frequency.

As used herein, “metabolic maturity,” refers to a the enhance metabolic properties or functional phenotype of a cell as described herein. Metabolic maturity can be determined by one or more of the following markers compared to a reference level selected from the group consisting of: increased activity of mitochondrial function, increased fatty acid metabolism, increased oxygen consumption rate (OCR), increased phosphorylated ACC levels or activity, increased level or activity of fatty acid binding protein (FABP), increased level or activity of pyruvate dehydrogenase kinase-4 (PDK4), increased mitochondrial respiratory capacity, increased mitochondrial volume, and increased levels of mitochondrial DNA.

As used herein, “contractile maturity,” refers to a the enhance contractile properties or functional phenotype of a cardiomyocyte as described herein. Contractile maturity can be determined by one or more of the following markers compared to a reference level selected from the group consisting of: decreased beat frequency, increased contractile force, increased level or activity of α-myosin heavy chain (α-MHC), increased level or activity of sarcomeres, decreased circularity index, increased level or activity of troponin, increased level or activity of titin N2b, increased cell area, and increased aspect ratio.

As used herein, “treating” or “administering” are used interchangeably in the context of the placement of an agent (e.g. a small molecule) described herein, into a subject, by a method or route which results in at least partial localization of the agent at a desired site, such as the gastrointestinal tract, heart, brain, or a region thereof, such that a desired effect(s) is produced (e.g., increased AMPK level or activity). The agent described herein can be administered by any appropriate route which results in delivery to a desired location in the subject. The half-life of the agent after administration to a subject can be as short as a few minutes, hours, or days, e.g., twenty-four hours, to a few days, to as long as several years, i.e., long-term. In some embodiments of any of the aspects, the term “treatment” refers to the administration of a pharmaceutical composition comprising one or more agents or contacting a cell, tissue, or organ with an agent. The administering can be done by contacting the cells of interest, direct injection (e.g., directly administered to a target cell or tissue), subcutaneous injection, muscular injection, oral, or nasal delivery to the subject in need thereof. Administering can be transient, local, or systemic.

As used herein, the term “adenosine monophosphate-activated protein kinase” or “AMPK” refers to a ubiquitously expressed heterotrimeric kinase. AMPK is a serine-threonine kinase that is allosterically activated by increases in the ratio of [AMP] or [ADP] to [ATP]. AMPK regulates several pathways involved in fatty acid and glucose transport into the cell, increases glycolytic flux, and enhances mitochondrial entry of fatty acyl carnitine. Generally, AMPK activation is associated with inhibition of energy consumption by a cell (e.g., decreasing ATP consuming pathways), and increasing glucose uptake, lipolysis, and mitochondrial metabolism. Sequences for AMPK are known for a number of species, e.g., human (NCBI Gene ID: 5562) polypeptide and mRNA (e.g., NCBI Reference Sequence: NP_006242.5 and NCBI Reference Sequence: NM_006251.5). AMPK can refer to human AMPK, including naturally occurring variant molecules, genetically engineered AMPK, and alleles thereof. AMPK refers to the mammalian AMPK of, e.g., mouse, rat, rabbit, dog, cat, cow, horse, pig, and the like. The amino acid sequence of human AMPK is shown in SEQ ID NO: 1. The human mRNA transcript sequence is shown in SEQ ID NO: 2.

As used herein, the term “AICAR” or “5-amino-4-imidazolecarboxamide riboside-1-β-D-ribofuranoside” refers to an activator of AMPK. AICAR is taken up by adenosine transporters and subsequently phosphorylated by adenosine kinase to ZMP (5-aminoimidazole-4-carboxamide-1-β-D-furanosyl 5′-monophosphate), which in turn mimics AMP to activate AMPK.

An “activator” or “agent” as used herein is a chemical molecule of synthetic or biological origin. In the context of the present invention, an activator is generally a molecule that can be used in a pharmaceutical composition.

As used herein, “activator of adenosine monophosphate-activated protein kinase (AMPK)”, is any agent, compound, small molecule, nucleic acid, polypeptide, etc. that increases the activity or levels of AMPK directly or indirectly. Non-limiting examples of activators for AMPK include AICAR, ZMP, or any derivative thereof, including those disclosed in U.S. Pat. No. 5,777,100, hereby incorporated by reference herein and prodrugs or precursors of AICAR (such as those disclosed in U.S. Pat. No. 5,082,829, hereby incorporated by reference herein).

As used herein, the terms “disease” or “disorder” refers to a disease, syndrome, or disorder, partially or completely, directly or indirectly, caused by one or more abnormalities in the genome, physiology, or behavior, or health of a subject.

The disease or disorder can be a cardiac disease or disorder. As used herein, the term, “cardiac disease” refers to a disease that affects the circulatory system of a subject. Non-limiting examples of cardiac diseases include arrhythmogenic right ventricular dysplasia (ARVD), cardiomyopathy, cardiac arrhythmia, cardiomyopathy, long QT syndrome, catecholaminergic polymorphic ventricular tachycardia (CPVT), Barth syndrome, and Duchenne muscular dystrophy. The disease or disorder can be a neurological disease or disorder.

As used herein, the term, “neurological disease” refers to a disease that affects the central or peripheral nervous system of a subject. Non-limiting examples of neurological diseases includes Alzheimer's disease, Parkinson's disease, Down syndrome, dementia, multiple sclerosis, and amyotrophic lateral sclerosis (ALS).

The terms “patient”, “subject” and “individual” are used interchangeably herein, and refer to an animal, particularly a human, to whom treatment, including prophylactic treatment is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment of any of the aspects, the subject is human. In another embodiment, of any of the aspects, the subject is an experimental animal or animal substitute as a disease model. In another embodiment, of any of the aspects, the subject is a domesticated animal including companion animals (e.g., dogs, cats, rats, guinea pigs, hamsters etc.). A subject can have previously received a treatment for a disease, or has never received treatment for a disease. A subject can have previously been diagnosed with having a disease, or has never been diagnosed with a disease.

As used herein, a “substrate” refers to a structure, comprising a biocompatible material that provides a surface suitable for adherence and proliferation of cells. A nanopatterned substrate can further provide mechanical stability and support. A nanopatterned substrate can be in a particular shape or form so as to influence or delimit a three-dimensional shape or form assumed by a population of proliferating cells. Such shapes or forms include, but are not limited to, films (e.g. a form with two-dimensions substantially greater than the third dimension), ribbons, cords, sheets, flat discs, cylinders, spheres, 3-dimensional amorphous shapes, etc. The substrate can be nanopatterned or micropatterned to permit the formation of engineered tissues on the substrate.

As used herein, the term “transplanting” is used in the context of the placement of cells, e.g. stem cells, cardiomyocytes, and/or neurons, as described herein into a subject, by a method or route which results in at least partial localization of the introduced cells at a desired site, such as a site of injury or repair, such that a desired effect(s) is produced. The cells e.g. cardiomyocytes or neurons, or their differentiated progeny (e.g. cardiac fibroblasts etc.) and cardiomyocytes or neurons can be implanted directly to the heart or spinal cord, or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment. As one of skill in the art will appreciate, long-term engraftment of the cardiomyocytes is desired as cardiomyocytes and neurons as they do not proliferate to an extent that the heart or spinal cord can heal from an acute injury comprising cell death. In other embodiments, the cells can be administered via an indirect systemic route of administration, such as an intraperitoneal or intravenous route.

As used herein, “in vitro-differentiated neurons” refers to neurons that are generated in culture, typically via step-wise differentiation from a precursor cell such as a human embryonic stem cell, an induced pluripotent stem cell, an early ectodermal cell or a neuronal progenitor cell.

As used herein, “amyloid beta” or “Aβ” refers to a neurotoxic polypeptide containing about 40 amino acid residues. It is produced by enzymatic cleavage of a larger precursor protein, beta-amyloid precursor protein, which is encoded by a gene on human chromosome 21, and is found in the brains of individuals suffering from Alzheimers disease in deposits known as senile plaques, among others. The Aβ described herein can be Aβ₁₋₄₂ or Aβ₁₋₄₀.

As used herein, “Tau protein” refers to a protein expressed in neurons that stabilizes microtubules. Tau is a phosphoprotein with 79 potential Serine (Ser) and Threonine (hr) phosphorylation sites on the longest tau isoform. The phosphorylated form of tau, as used herein as “phosphorylated tau protein” is a hallmark of Alzheimers disease. The accumulation of hyperphosphorylated tau in neurons can lead to the neurofibrillary degeneration.

The term “agent” or “activator” as used herein means any compound or substance such as, but not limited to, a small molecule, nucleic acid, polypeptide, peptide, drug, ion, etc. An “agent” can be any chemical, entity or moiety, including without limitation, synthetic and naturally-occurring proteinaceous and non-proteinaceous entities. In some embodiments of any of the aspects, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased,” “increase,” “increases,” or “enhance” or “activate” are all used herein to generally mean an increase of a property, level, or other parameter by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level. For example, increasing activity can refer to activating AMPK or increasing levels of AMPK directly or indirectly.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with a disease, or a biological sample that has not been contacted with an agent or composition disclosed herein).

As used herein, an “appropriate control” refers to an untreated, otherwise identical cell or population (e.g., a biological sample that was not contacted by an agent or composition described herein, or not contacted in the same manner, e.g., for a different duration, as compared to a non-control cell).

As used herein, the term “phenotypic characteristic,” as applied to in vitro differentiated cells (e.g., cardiomyocytes or neurons), or culture of in vitro-differentiated cells, refers to any of the parameters described herein as measures of cell function. A “change in a phenotypic characteristic” as described herein is indicated by a statistically significant increase or decrease in a functional property with respect to a reference level or appropriate control.

As used herein, the term “contacting” when used in reference to a cell, tissue, or organ, encompasses both introducing or administering an agent, surface, hormone, etc. to the cell, tissue, or organ in a manner that permits physical contact of the cell with the agent, surface, hormone etc., and introducing an element, such as a genetic construct or vector, that permits the expression of an agent, such as a miRNA, polypeptide, or other expression product in the cell. It should be understood that a cell genetically modified to express an agent, is “contacted” with the agent, as are the cell's progeny that express the agent.

The term “statistically significant” or “significantly” refers to statistical significance and generally means a two standard deviation (2SD) or greater difference.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean±1%.

### Cell Preparations

The methods and compositions described herein can use cardiomyocytes and neurons differentiated in vitro, e.g., from embryonic stem cells, pluripotent stem cells, such as induced pluripotent stem cells, or other stem cells that permit such differentiation. The following describes various stem cells that can be used to prepare in vitro-differentiated cardiomyocytes and neurons for use in the compositions and methods described herein.

Stem cells are cells that retain the ability to renew themselves through mitotic cell division and can differentiate into more specialized cell types. Three broad types of mammalian stem cells include: embryonic stem (ES) cells that are found in blastocysts, induced pluripotent stem cells (iPSCs) that are reprogrammed from somatic cells, and adult stem cells that are found in adult tissues. Other sources of stem cells can include amnion-derived or placental-derived stem cells. Pluripotent stem cells can differentiate into cells derived from any of the three germ layers.

Cardiomyocytes and neurons useful in the compositions and methods described herein can be differentiated from both embryonic stem cells and induced pluripotent stem cells, among others.

In one embodiment, the compositions and methods provided herein use human cardiomyocytes and/or neurons differentiated from embryonic stem cells. Alternatively, in some embodiments, the compositions and methods provided herein do not encompass generation or use of human cardiogenic cells and/or neurons made from cells taken from a viable human embryo.

Embryonic stem cells: Embryonic stem cells and methods for their retrieval are described, for example, in Trounson A. O. Reprod. Fertil. Dev. (2001) 13: 523, Roach M L Methods Mol. Biol. (2002) 185: 1, and Smith A. G. Annu Rev Cell Dev Biol (2001) 17:435. The term “embryonic stem cell” is used to refer to the pluripotent stem cells of the inner cell mass of the embryonic blastocyst (see e.g., U.S. Pat. Nos. 5,843,780, 6,200,806). Such cells can similarly be obtained from the inner cell mass of blastocysts derived from somatic cell nuclear transfer (see, for example. U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that the cell can be distinguished from other cells. Exemplary distinguishing embryonic stem cell characteristics include, without limitation, gene expression profile, proliferative capacity, differentiation capacity, karyotype, responsiveness to particular culture conditions, and the like. Markers of embryonic stem cells include, for example, any one or any combination of Oct3, Nanog, SOX2, SSEA1, SSEA4 and TRA-1-60.

Cells derived from embryonic sources can include embryonic stem cells or stem cell lines obtained from a stem cell bank or other recognized depository institution. Other means of producing stem cell lines include methods comprising the use of a blastomere cell from an early stage embryo prior to formation of the blastocyst (at around the 8-cell stage). Such techniques use, for example, single cells removed in the pre-implantation genetic diagnosis technique routinely practiced in assisted reproduction clinics. The single blastomere cell is co-cultured with established ES-cell lines and then separated from them to form fully competent ES cell lines.

Undifferentiated embryonic stem (ES) cells are easily recognized by those skilled in the art, and typically appear in the two dimensions of a microscopic view as colonies of cells with high nuclear/cytoplasmic ratios and prominent nucleoli. Markers of embryonic stem cells include, for example, anyone or any combination of Oct3, Nanog, SOX2, SSEA1, SSEA4 and TRA-1-60. In some embodiments, the human cardiomyocytes and/or neurons described herein are not derived from embryonic stem cells or any other cells of embryonic origin.

Induced Pluripotent Stem Cells (iPSCs): In some embodiments, the compositions and methods described herein utilize cardiomyocytes and/or neurons that are differentiated in vitro from induced pluripotent stem cells. An advantage of using iPSCs to generate cardiomyocytes and/or neurons for the compositions and methods described herein is that, if so desired, the cells can be derived from the same subject to which the desired human cardiomyocytes or neurons are to be administered. That is, a somatic cell can be obtained from a subject, reprogrammed to an induced pluripotent stem cell, and then re-differentiated into a human cardiomyocyte or neuron to be administered to the subject (i.e., autologous cells). Since the cardiomyocytes and/or neurons (or their differentiated progeny) are essentially derived from an autologous source, the risk of engraftment rejection or allergic responses is reduced compared to the use of cells from another subject or group of subjects. While this is an advantage of iPS cells, in alternative embodiments, the cardiomyocytes and/or neurons useful for the methods and compositions described herein are derived from non-autologous sources (i.e., allogenic cells). In addition, the use of iPSCs negates the need for cells obtained from an embryonic source.

Although differentiation is generally irreversible under physiological contexts, several methods have been developed in recent years to reprogram somatic cells to induced pluripotent stem cells. Exemplary methods are known to those of skill in the art and are described briefly herein below.

Reprogramming is a process that alters or reverses the differentiation state of a differentiated cell (e.g., a somatic cell). Stated another way, reprogramming is a process of driving the differentiation of a cell backwards to a more undifferentiated or more primitive type of cell. It should be noted that placing many primary cells in culture can lead to some loss of fully differentiated characteristics. However, simply culturing such cells included in the term differentiated cells does not render these cells non-differentiated cells or pluripotent cells. The transition of a differentiated cell to pluripotency requires a reprogramming stimulus beyond the stimuli that lead to partial loss of differentiated character when differentiated cells are placed in culture. Reprogrammed cells also have the characteristic of the capacity of extended passaging without loss of growth potential, relative to primary cell parents, which generally have capacity for only a limited number of divisions in culture.

The cell to be reprogrammed can be either partially or terminally differentiated prior to reprogramming. Thus, cells to be reprogrammed can be terminally differentiated somatic cells, as well as adult or somatic stem cells.

In some embodiments, reprogramming encompasses complete reversion of the differentiation state of a differentiated cell (e.g., a somatic cell) to a pluripotent state or a multipotent state. In some embodiments, reprogramming encompasses complete or partial reversion of the differentiation state of a differentiated cell to an undifferentiated cell (e.g., an embryonic-like cell). Reprogramming can result in expression of particular genes by the cells, the expression of which further contributes to reprogramming. In certain embodiments described herein, reprogramming of a differentiated cell causes the differentiated cell to assume an undifferentiated state with the capacity for self-renewal and differentiation to cells of all three germ layer lineages. These are induced pluripotent stem cells (iPSCs or iPS cells).

Methods of reprogramming somatic cells into iPS cells are described, for example, in U.S. Pat. Nos. 8,129,187 B2; 8,058,065 B2; US Patent Application 2012/0021519 A1; Singh et al. Front. Cell Dev. Biol. (February, 2015); and Park et al., Nature 451: 141-146 (2008); which are incorporated by reference in their entireties. Specifically, iPSCs are generated from somatic cells by introducing a combination of reprogramming transcription factors. The reprogramming factors can be introduced as, for example, proteins, nucleic acids (mRNA molecules, DNA constructs or vectors encoding them) or any combination thereof. Small molecules can also augment or supplement introduced transcription factors. While additional factors have been determined to affect, for example, the efficiency of reprogramming, a standard set of four reprogramming factors sufficient in combination to reprogram somatic cells to an induced pluripotent state includes Oct4 (Octamer binding transcription factor-4), SOX2 (Sex determining region Y)-box 2, Klf4 (Kruppel Like Factor-4), and c-Myc. Additional protein or nucleic acid factors (or constructs encoding them) including, but not limited to LIN28+Nanog, Esrrb, Pax5 shRNA, C/EBPα, p53 siRNA, UTF1, DNMT shRNA, Wnt3a, SV40 LT(T), hTERT) or small molecule chemical agents including, but not limited to BIX-01294, BayK8644, RG108, AZA, dexamethasone, VPA, TSA, SAHA, PD0325901+CHIR99021(2i) and A-83-01 have been found to replace one or the other reprogramming factors from the basal or standard set of four reprogramming factors, or to enhance the efficiency of reprogramming.

Reprogrammed somatic cells as disclosed herein can express any number of stem cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; β-III-tubulin; α-smooth muscle actin (α-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fthl17; Sal14; undifferentiated embryonic cell transcription factor (Utfl); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; other general markers for pluripotency, etc. Other markers can include Dnmt3L; Sox15; Stat3; Grb2; β-catenin, and Bmi1. Such cells can also be characterized by the down-regulation of markers characteristic of the somatic cell from which the induced pluripotent stem cell is derived.

The specific approach or method used to generate pluripotent stem cells from somatic cells (e.g., any cell of the body with the exclusion of a germ line cell; fibroblasts, etc.) is not critical to the claimed invention. Thus, any method that re-programs a somatic cell to the pluripotent phenotype would be appropriate for use in the methods described herein.

The efficiency of reprogramming (i.e., the number of reprogrammed cells) derived from a population of starting cells can be enhanced by the addition of various small molecules as shown by Shi, Y., et al. (2008) Cell-Stem Cell 2:525-528, Huangfu, D., et al. (2008) Nature Biotechnology 26(7):795-797, and Marson, A., et al. (2008) Cell-Stem Cell 3:132-135. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), among others.

To confirm the induction of pluripotent stem cells for use with the methods described herein, isolated clones can be tested for the expression of one or more stem cell markers. Such expression in a cell derived from a somatic cell identifies the cells as induced pluripotent stem cells. Stem cell markers can include but are not limited to SSEA3, SSEA4, CD9, Nanog, Oct4, Fbx15, Ecat1, Esg1, Eras, Gdf3, Fgf4, Cripto, Dax1, Zpf296, Slc2a3, Rex1, Utfl, and Nat1, among others. In one embodiment, a cell that expresses Nanog and SSEA4 is identified as pluripotent. Methods for detecting the expression of such markers can include, for example, RT-PCR and immunological methods that detect the presence of the encoded polypeptides, such as Western blots or flow cytometric analyses. Intracellular markers may be best identified via RT-PCR, while cell surface markers are readily identified, e.g., by immunocytochemistry.

The pluripotent stem cell character of isolated cells can be confirmed by tests evaluating the ability of the iPSCs to differentiate to cells of each of the three germ layers. As one example, teratoma formation in nude mice can be used to evaluate the pluripotent character of the isolated clones. The cells are introduced to nude mice and histology and/or immunohistochemistry using antibodies specific for markers of the different germ line lineages is performed on a tumor arising from the cells. The growth of a tumor comprising cells from all three germ layers, endoderm, mesoderm and ectoderm further indicates or confirms that the cells are pluripotent stem cells.

Adult Stem Cells: Adult stem cells are stem cells derived from tissues of a post-natal or post-neonatal organism or from an adult organism. An adult stem cell is structurally distinct from an embryonic stem cell not only in markers it does or does not express relative to an embryonic stem cell, but also by the presence of epigenetic differences, e.g. differences in DNA methylation patterns. It is contemplated that cardiomyocytes and/or neurons differentiated from adult stem cells can also be used for the methods described herein. Methods of isolating adult stem cell are described for example, in U.S. Pat. No. 9,206,393 B2; and US Application No. 2010/0166714 A1; which are incorporated herein by reference in their entireties.

### In Vitro-Differentiation

The methods and compositions described herein use in vitro-differentiated cardiomyocytes and neurons. Methods for the differentiation of either cell type from ESCs or iPSCs are described in, e.g., LaFlamme et al., Nature Biotech 25:1015-1024 (2007), which describes the differentiation of cardiomyocytes, and Yuan et al. PloS One 6: e17540 (2011), which describes the differentiation of neurons. The contents of each of these references in regard to the differentiation of pluripotent stem cells to the respective cell types are incorporated herein by reference in their entireties.

With regard to cardiomyocytes, the basic step-wise differentiation of ESCs or iPSCs to cardiomyocytes proceeds in the following order: ESC or iPSC>cardiogenic mesoderm>cardiac progenitor cells>cardiomyocytes. See e.g., Lian et al. Nat. Prot. (2013); US published patent application No. 2017/0058263 A1; 2008/0089874 A1; 2006/0040389 A1; U.S. Pat. Nos. 10,155,927 B2; 9,994,812 B2; and 9,663,764 B2, the contents of each of which are incorporated herein by reference their entireties.

A number of protocols for differentiating ESCs and iPSCs to cardiomyocytes can be used. For example, agents can be added or removed from cell culture media to direct differentiation to cardiomyocytes in a step-wise fashion. Non-limiting examples of factors and agents that can promote cardiomyocyte differentiation include small molecules (e.g., Wnt inhibitors, GSK3 inhibitors), polypeptides (e.g., growth factors), nucleic acids or vectors encoding them, and patterned substrates (e.g., nanopatterns). The addition of growth factors necessary in cardiovascular development, including but not limited to fibroblast growth factor 2 (FGF2), transforming growth factor β (TGFβ) superfamily growth factors—Activin A and BMP4, vascular endothelial growth factor (VEGF), and the Wnt inhibitor DKK-1, can also be beneficial in directing differentiation along the cardiac lineage. Additional examples of factors and conditions that help promote cardiomyocyte differentiation include but are not limited to B27 supplement lacking insulin, cell-conditioned media, external electrical pacing, and nanopatterned substrates, among others. Example 1 herein below demonstrates a representative approach for the generation of cardiomyocytes from iPS cells following the method of Yang et al., J. Mol. Cell. Cardiol. 72: 296-304 (2014), the contents of which are also incorporated herein by reference in their entirety. Cardiomyocytes have characteristic morphology and marker expression and also spontaneously beat in culture. Additional metabolic, structural and functional characteristics are described elsewhere herein and are measured in the Examples, but at a minimum, cardiomyocytes will express cardiac Troponin T (cTnT).

For the in-vitro differentiation of neurons, the basic step-wise differentiation of ESCs or iPSCs to neurons proceeds as follows: ESC or iPSC>neural ectoderm>neural progenitor cells>neurons. See e.g., Yuan et al., PloS One. 6: e17540 (2011); Israel et al., Nature. 2012; 482:216-20.; and Yeo et al., PLoS Comput Biol. 2007; 3:1951-1967, which are incorporated herein by reference in their entireties.

Any of a number of protocols for differentiating ESCs and iPSCs to neurons can be used. Non-limiting examples of factors and agents that can promote neural differentiation include small molecules (e.g., SB431542), polypeptides (e.g., growth factors, BDNF), nucleic acids and vectors encoding them. Differentiation can include the addition of growth factors necessary in neural development, including but not limited to Noggin, SB431542, the withdrawal of bovine fibroblast growth factor (bFGF), and the addition of brain-derived neurotrophic factor (BDNF), glial cell line-derived neurotrophic factor (GDNF), and/or dibutyryl cyclic AMP (dbCAMP). By way of example only, cells that express Sox1 and nestin can be readily differentiated into neurons upon withdrawal of FGF-2. Example 1 herein below demonstrates a representative approach for the generation of neurons from iPS cells following the method of Yuan et al., PloS One. 6: e17540 (2011). Neurons have characteristic morphology and marker expression. Markers for neurons include, but are not limited to βIII tubulin, synaptophysin, synapsin, GABA, Map2a/b and tyrosine hydroxylase. NeuN (Neuronal nuclei protein, FOX3) is expressed in nearly all neuronal types and provides a measure of neuronal maturation, with levels increasing with increasing degree of maturity. Additional metabolic, structural and functional characteristics are described elsewhere herein and are measured in the Examples, but at a minimum, neurons will express βIII tubulin (also known as TuJI).

In some embodiments, the desired cells (e.g., in vitro-differentiated cardiomyocytes or neurons) are an enriched population of cells; that is, the percentage of human in vitro-differentiated cardiomyocytes or neurons (e.g., percent of cells) in a population of cells is at least 10% of the total number of cells in the population. For example, an enriched population comprises at least 15% definitive cardiomyocytes or neurons, at least 20%, at least 30%, at least 40%, at least 50%, at least 60%, at least 70%, at least 80%, at least 90%, at least 95%, at least 99% or even 100% of the population comprises human in vitro-differentiated cardiomyocytes or neurons. In some embodiments, a population of cells comprises at least 100 cells, at least 500 cells, at least 1000 cells, at least 1×10⁴ cells, at least 1×10⁵ cells, at least 1×10⁶ cells, at least 1×10⁷ cells, at least 1×10⁸ cells, at least 1×10⁹ cells, at least 1×10¹⁰ cells, at least 1×10¹¹ cells, at least 1×10¹² cells, or more.

The stem cells or cardiomyocyte/neural progenitors can be cultured on a mouse embryonic fibroblast (MEF) feeder layer of cells, Matrigel®, collagenase IV, or any other matrix or scaffold that substantially promotes in-vitro differentiation of the desired cell type. Furthermore, specific cell types of in-vitro differentiated cardiomyocytes or neurons can be isolated or enriched for by a variety of methods including but not limited to cell sorting, such as fluorescent activated cell sorting (FACS) or magnetic activated cell sorting (MACS), microfluidic devices, buoyancy activated cell sorting, or a microraft array, panning methods, magnetic particle selection, particle sorter selection and other methods known to persons skilled in the art, including density separation (Xu et al. (2002) Circ. Res. 91:501; U.S.S.N. 20030022367) and separation based on other physical properties (Doevendans et al. (2000) J. Mol. Cell. Cardiol. 32:839-851). Negative selection can be performed, including selecting and removing cells with undesired markers or characteristics, for example fibroblast markers, epithelial cell markers etc.

For example, undifferentiated ES cells express genes that can be used as markers to detect the presence of undifferentiated cells. Exemplary ES cell markers include stage-specific embryonic antigen (SSEA)-3, SSEA-4, TRA-I-60, TRA-1-81, alkaline phosphatase or those described in e.g., U.S.S.N. 2003/0224411; or Bhattacharya (2004) Blood 103(8):2956-64, each herein incorporated by reference in their entirety. Exemplary markers expressed on cardiac progenitor cells include, but are not limited to, TMEM88, GATA4, ISL1, MYL4, and NKX2-5. Such markers can be assessed or used to remove or determine the presence of undifferentiated or progenitor cells in, e.g., a population of in vitro-differentiated cardiomyocytes. Similarly, the presence of markers of undifferentiated cells, whether embryonic markers or otherwise, can be used to evaluate populations of cardiomyocytes and/or neurons useful in the methods and compositions described herein.

Exemplary markers expressed by cardiomyocytes include, but are not limited to, NKX2-5, MYH6, MYL7, TBX5, ATP2a2, RYR2, and cTnT.

Exemplary markers expressed by neurons include, but are not limited to, amyloid beta (Aβ), neuronal nuclei (NeuN), nestin, SOX2, ABCG2, FGF R4, Frizzle-9, GABA, Choline acetyltransferase (ChAT), tyrosine hydroxylase (TH), neuron specific enolaste (NSE), and microtubule-associated protein 2 (MAP-2).

Monitoring Function and Maturity of Cardiomyocytes and/or Neurons

The methods and compositions described herein use in vitro-differentiated cardiomyocytes and/or neurons prepared, for example, as described above and in the Examples herein below. As also described above, the maturity of the in vitro-differentiated cells can be promoted or enhanced by treatment or contacting of the cells with an activator of AMPK.

The degree of cellular maturation or maturity can be determined by a number of parameters such as electrical maturity of a cell, metabolic maturity of a cell, or contractile maturity of a cardiomyocyte.

With regard to cardiomyocytes, electrical maturity is determined by one or more of the following markers as compared to a reference level: increased gene expression of an ion channel gene, increased sodium current density, increased inwardly-rectifying potassium channel current density, decreased action potential frequency, decreased calcium wave frequency, and decreased field potential frequency. Non-limiting examples of cardiac ion channel genes include SCN5A, KCNJ2, KCNJ5, KCNJ11, KCNJ8, KCNH2, KCNE1, KCNQ1, KCNE2, CACNA1C, SCN1B, SCN10A, CACNA1S, and KCNA5. Methods of measuring gene expression are known in the art, e.g., RT-PCR and immunodetection methods, such as Western blotting and immunocytochemistry, among others.

Mature cardiomyocytes have functional ion channels that permit the synchronization of cardiac muscle contraction. The electrical function of cardiomyocytes can be measured by a variety of methods. Non-limiting examples of such methods include whole cell patch clamp (manual or automated), multielectrode arrays, field potential stimulation, calcium imaging and optical mapping, among others. Cardiomyocytes can be electrically stimulated during whole cell current clamp or field potential recordings to produce an electrical and/or contractile responses. Measurement of field potentials and biopotentials of cardiomyocytes can be used to determine or monitor their differentiation stage and cell maturity.

Metabolic maturity of in vitro-differentiated cardiomyocytes is determined by one or more of the following markers as compared to a reference level: increased activity of mitochondrial function, increased fatty acid metabolism, increased oxygen consumption rate (OCR), increased phosphorylated ACC levels or activity, increased level or activity of fatty acid binding protein (FABP), increased level or activity of pyruvate dehydrogenase kinase-4 (PDK4), increased mitochondrial respiratory capacity, increased mitochondrial volume, and increased levels of mitochondrial DNA.

Metabolic assays can be used to determine the differentiation stage and cell maturity of the stem cell-derived cardiomyocytes as described herein. Non-limiting examples of metabolic assays include cellular bioenergetics assays (e.g., Seahorse Bioscience XF Extracellular Flux Analyzer), and oxygen consumption tests. Specifically, cellular metabolism can be quantified by oxygen consumption rate (OCR), OCR trace during a fatty acid stress test, maximum change in OCR, maximum change in OCR after FCCP addition, and maximum respiratory capacity, among other parameters.

Furthermore, a metabolic challenge or lactate enrichment assay can provide a measure of stem cell-derived cardiomyocyte maturity or a measure of the effects of various treatments of such cells. Mammalian cells generally use glucose as their main energy source. However, cardiomyocytes are capable of energy production from different sources such as lactate or fatty acids. In some embodiments, lactate-supplemented and glucose-depleted culture medium, or the ability of cells to use lactate or fatty acids as an energy source is useful to identify mature cardiomyocytes and variations in their function.

Contractile maturity is determined by one or more of the following markers as compared to a reference level: decreased beat frequency, increased contractile force, increased level or activity of α-myosin heavy chain (α-MHC), increased level or activity of sarcomeres, decreased circularity index, increased level or activity of troponin, increased level or activity of titin N2b, increased cell area, and increased aspect ratio. Contractility can be measured by optical tracking methods such as video analysis. For video tracking methods, contraction (or systole) of the cardiomyocytes is considered to be the point in time and space where the cell or cardiac tissue is at the shortest length. Relaxation (diastole) is considered to be the point in time or space where the cell or cardiac tissue is at the largest length. These parameters are determined by measuring displacement of tissues or single cells.

In addition to optical tracking, impedimetric measurements can also be performed. For example, the cardiomyocytes described herein can have contractility or beat rate measurements determined by xCelligence™ real time cell analysis (Acea Biosciences, Inc., San Diego, Calif.).

A useful parameter to determine cardiomyocyte function is beat rate. The frequency of the contraction, beat rate, change in beat interval (ΔBI), or beat period, can be used to determine stem cell differentiation stage and stem cell-derived cardiomyocyte maturity. Beat rate can be measured by optical tracking. The beat rate is typically elevated in fetal cardiomyocytes and is reduced as cardiomyocytes develop. During disease states the change in beat rate can be variable and lack a constant frequency due to electrophysiological or structural instability.

Another useful parameter to determine cardiomyocyte function and contractile maturity is contractile force. Optical tracking can be used to determine the displacement of cardiac tissue as the tissue beats in culture. Force tracing of paced cardiac tissue over time can be calculated with custom software. Force output of the cardiac tissues can be increased using pharmaceuticals known in the art (e.g., isoproterenol) to measure the relative changes in contractile function with each dose.

For neurons, markers specific for neural maturity can include increased levels or activity of PPARα, increased levels or activity of TFAM, increased levels or activity of PDK4, increased levels or activity of NeuN, reduced levels or activity of amyloid beta (Aβ), reduced levels or activity of phosphorylated Tau protein, increased activity of mitochondrial function, increased fatty acid metabolism, increased levels and activity of ion channel genes or the channels themselves, and increased levels of mitochondrial DNA when compared to an appropriate control. Non-limiting examples of neural ion channel genes include SCN1A, SCN2A, SCN3A, SCN8A, KCNA1, KCNA2, KCNA3, KCNA4, KCNA6, KCNB1, KCNB2, KCNC1, KCND1, KCNQ1, KCNQ2, KCNQ3, KCNQ5, KCNV1, KCNH1, KCNF1, CACNA1C, CACNA1D, CACNA1A, CACNA1B, CACNA1E, CACNA1G, CACNA1H, and CACNA1I.

In addition, the electrical and metabolic function of neurons can be measured by a variety of methods as described above for cardiomyocytes.

### Activators of AMPK

The methods and compositions described herein employ an activator of adenosine monophosphate-activated protein kinase (AMPK) for the maturation of in vitro differentiated cells. Assays for AMPK activity are known in the art, and include, for example, the assay described by Lim et al., Methods Enzymol. 514: 271-287 (2012), which is incorporated herein by reference. Briefly, the assay involves immunoprecipitating AMPK from the tissue or cells of interest, followed by quantification of its enzyme activity using labeled ATP in the presence of a substrate. A key physiological substrate is acetyl-CoA carboxylase, and this substrate can be used in an in vitro assay as well, with detection of phosphorylation through radiolabeled ATP providing a readout of AMPK activity. Peptide substrates are also known. A FRET-based assay is described by Wilson et al., Bio-Protocol 9, Issue 8, 2019, which is incorporated herein by reference. A different FRET-based assay, which measures AMPK conformational state is described by Pelosse et al. Nature Commun. 10: 1038 (2019), and is incorporated herein by reference. AMPK Thr(172) phosphorylation, detected, for example, via immunoassay can also be used as a surrogate marker for AMPK activity. ThermoFisher Scientific sells an ELISA-based kit for measuring Thr(172) phosphorylation of human AMPK—see Catalog #KHO0651. Any of these assays can be used to determine, for example, whether a given agent, whether small molecule, polypeptide, polynucleotide or other, can activate AMPK activity. Briefly, an assay run separately with and without a candidate agent can provide a readout of the effect of the candidate agent on AMPK activity. Such an assay conducted, for example, with varying amounts of the candidate agent can provide a curve from which the effective concentration range of the agent can be determined.

As an alternative, or in addition to the assays that measure AMPK enzyme activity, if the agent promotes the expression of AMPK, or if the agent is or encodes an AMPK polypeptide, e.g., a wild-type or constitutively active AMPK polypeptide or enzymatically-active fragment thereof, measurement of the level of AMPK protein can provide a readout of AMPK activity or activation.

In some embodiments, the activator of AMPK comprises a small molecule, a polypeptide, a nucleic acid encoding a polypeptide or a vector encoding a polypeptide.

As used herein, the term “small molecule” refers to a organic or inorganic molecule, either natural (i.e., found in nature) or non-natural (i.e., not found in nature), which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. Examples of small molecules that occur in nature include, but are not limited to, taxol, dynemicin, and rapamycin. Examples of “small molecules” that are synthesized in the laboratory include, but are not limited to, compounds described in Tan et al., (“Stereoselective Synthesis of over Two Million Compounds Having Structural Features Both Reminiscent of Natural Products and Compatible with Miniaturized Cell-Based Assays” J. Am. Chem. Soc. 120:8565, 1998; incorporated herein by reference). In certain other preferred embodiments, natural-product-like small molecules are utilized.

In one embodiment, the small molecule is 5-aminoimidizole-4-carboxamide riboside (AICAR) or a derivative thereof that activates AMPK. In some embodiments, the activator includes analogs of AICAR (such as those disclosed in U.S. Pat. No. 5,777,100, hereby incorporated by reference herein) and prodrugs or precursors of AICAR (such as those disclosed in U.S. Pat. No. 5,082,829, hereby incorporated by reference herein), which increase the bioavailability of AICAR.

In one embodiment, a derivative is a molecule structurally similar to AICAR or ZMP which activates AMPK. In the normal course of cellular metabolism, AMPK activity is regulated by the ratio of ADP:ATP or AMP:ATP. For example, increases in the ratio of AMP:ATP allow for AMP interaction with the gamma (γ)-subunit of AMPK. When AMP is bound to the allosteric site on the γ-subunit of AMPK, this allows for phosphorylation of the α-subunit by other kinases. When residue T172 of AMPK's alpha (α)-subunit is phosphorylated e.g., by LKB1 and CAMKKβ, AMPK is activated. Thus, derivatives of AICAR or ZMP that mimic the regulatory activity of AMP are potentially useful in the compositions and methods described herein. The crystal structure of the regulatory fragment of human AMPK complexed with AMP has been solved. See, e.g., RCSB Protein Data Bank accession 2V8Q. Using those crystal coordinates and molecular modeling software, one can determine which variants of AMP or variants of AICAR and/or ZMP, including but not limited to those described in U.S. Pat. No. 5,777,100 or 5,082,829, for example, will likely bind AMPKγ subunit at the allosteric site and induce the desired conformational change that activates the enzyme.

In some embodiments, the activator of AMPK is a nucleic acid encoding a polypeptide or a vector encoding a polypeptide.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures. For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues provided herein are preferred to be in the “L” isomeric form. However, residues in the “D” isomeric form may be substituted for any L-amino acid residue provided the desired properties of the polypeptide are retained.

As used herein, the term “nucleic acid” includes one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, without limitation, gDNA; hnRNA; mRNA; rRNA, tRNA, micro RNA (miRNA), small interfering RNA (siRNA), small nucleolar RNA (snORNA), small nuclear RNA (snRNA), and small temporal RNA (stRNA), and the like, and any combination thereof.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide (e.g. AMPK) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free host cells. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Another example of a non-integrative vector is a minicircle vector. Minicircle vectors are circularized vectors in which the plasmid backbone has been released leaving only the eukaryotic promoter and cDNA(s) that are to be expressed.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

In another embodiment of any of the aspects, AMPK is increased in the cell's genome using any genome editing system including, but not limited to, zinc finger nucleases, TALENS, meganucleases, and CRISPR/Cas systems. In one embodiment of any of the aspects, the genomic editing system used to incorporate the nucleic acid encoding one or more guide RNAs into the cell's genome is not a CRISPR/Cas system; this can prevent undesirable cell death in cells that retain a small amount of Cas enzyme/protein. It is also contemplated herein that either the Cas enzyme or the sgRNAs are each expressed under the control of a different inducible promoter, thereby allowing temporal expression of each to prevent such interference. The gene editing system can directly or indirectly modulate levels or activity of AMPK, e.g. by inhibiting transcriptional repressors that results in an increase in AMPK transcription.

In one embodiment, the treatment with an activator of AMPK is for at least two days, three days, four days, five days, six days, one week, or two weeks or more. In one embodiment, treatment is continued until a chosen marker or markers of maturity as known in the art or as described herein, whether, for example a protein marker or level thereof, or a functional marker, e.g., a metabolic marker, or a combination of protein and functional markers, reaches a level indicative of enhanced maturity relative to pre-treatment levels or indicative of a likelihood of improved transplant function.

In another embodiment, the activity of AMPK is increased by at least 20%, at least 30%, at least 40%, at least 50%, at least 60%, at least 70%, at least 80%, at least 90%, or more, e.g., at least 2-fold, at least 3-fold or more as compared to an appropriate control.

Amounts of AMPK activators effective to promote maturation of in vitro differentiated cardiomyocytes can vary depending upon the activator. For example, AICAR can be used in the range of about 50 micromolar to about 10 mM, e.g. about 50 micromolar to about 5 mM, about 50 micromolar to about 1 mM, about 100 micromolar to about 10 mM, about 100 micromolar to about 5 mM, about 100 micromolar to about 1 mM, about 200 micromolar to about 10 mM, about 200 micromolar to about 5 mM, about 200 micromolar to about 1 mM, about 300 micromolar to about 10 mM, about 300 micromolar to about 5 mM, about 300 micromolar to about 1 mM, about 400 micromolar to about 10 mM, about 400 micromolar to about 5 mM, about 400 micromolar to about 1 mM, about 500 micromolar to about 10 mM, about 500 micromolar to about 5 mM, about 500 micromolar to about 1 mM, about 600 micromolar to about 10 mM, about 600 micromolar to about 5 mM, about 600 micromolar to about 1 mM, about 700 micromolar to about 10 mM, about 700 micromolar to about 5 mM, about 700 micromolar to about 1 mM, about 800 micromolar to about 10 mM, about 800 micromolar to about 5 mM, about 800 micromolar to about 1 mM, about 900 micromolar to about 10 mM, about 900 micromolar to about 5 mM, about 900 micromolar to about 1 mM, about 1 mM to about 10 mM, about 2 mM to about 10 mM, about 3 mM to about 10 mM, about 4 mM to about 10 mM, about 5 mM to about 10 mM, about 6 mM to about 10 mM, about 7 mM to about 10 mM, about 8 mM to about 10 mM or about 9 mM to about 10 mM. Amounts of AICAR derivatives will generally be similar, depending upon the specific derivative and its effect on AMPK activity, although derivatives that act at lower concentrations relative to AICAR are specifically contemplated as being beneficial.

### Pharmaceutically Acceptable Carriers

The methods of administering matured human cardiomyocytes or neurons to a subject as described herein involve the use of therapeutic compositions comprising such cells. Therapeutic compositions contain a physiologically tolerable carrier together with the cell composition. In a preferred embodiment, the therapeutic composition is not substantially immunogenic when administered to a mammal or human patient for therapeutic purposes, unless so desired. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset, transplant rejection, allergic reaction, and the like. A pharmaceutically acceptable carrier will not promote the raising of an immune response to an agent with which it is admixed, unless so desired.

Cells for transplant can be formulated, for example, as a suspension, e.g., admixed in saline or other pharmaceutically acceptable isotonic carrier solution. Aqueous carriers can contain more than one buffer salt, as well as salts such as sodium and potassium chlorides, dextrose, polyethylene glycol and other solutes. Such a suspension can be injectable as is, or can be supplemented with or contain a matrix that improves consistency or other properties favoring engraftment of the administered cells. In one embodiment, an injectable matrix formulation provides a scaffold for the administered cells. Alternatively, cells can be placed or prepared on a scaffold that is then placed or implanted surgically, rather than by injection. Scaffolds and matrices suitable for such formulations are described herein below.

The cells and any other active ingredient can be mixed with excipients which are pharmaceutically acceptable and in amounts suitable for use in the therapeutic methods described herein. One of skill in the art will recognize that a pharmaceutically acceptable carrier to be used in with a cell composition will not include buffers, compounds, cryopreservation agents, preservatives, or other agents in amounts that substantially interfere with the viability of the cells to be delivered to the subject. A formulation comprising cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with cardiomyocytes or neurons as described herein using only routine experimentation.

### Scaffold Compostions

In one aspect, the cardiomyocytes and/or neurons described herein can be admixed with or grown in or on a preparation that provides a scaffold or nanopatterned substrate to support the cells. Such a scaffold or nanopatterned substrate can provide a physical advantage in securing the cells in a given location, e.g., after implantation, as well as a biochemical advantage in providing, for example, extracellular cues for the further maturation or, e.g., maintenance of phenotype until the cells are established.

Biocompatible synthetic, natural, as well as semi-synthetic polymers can be used for synthesizing polymeric particles that can be used as a scaffold material. In general, for the practice of the methods described herein, it is preferable that a scaffold biodegrades such that the cardiomyocytes and/or neurons can be isolated from the polymer prior to implantation or such that the scaffold degrades over time in a subject and does not require removal. Thus, in one embodiment, the scaffold provides a temporary structure for growth and/or delivery of cardiomyocytes and/or neurons to a subject in need thereof. In some embodiments, the scaffold permits human cells to be grown in a shape suitable for transplantation or administration into a subject in need thereof, thereby permitting removal of the scaffold prior to implantation and reducing the risk of rejection or allergic response initiated by the scaffold itself.

Examples of polymers which can be used include natural and synthetic polymers, although synthetic polymers are preferred for reproducibility and controlled release kinetics. Synthetic polymers that can be used include biodegradable polymers such as poly(lactide) (PLA), poly(glycolic acid) (PGA), poly(lactide-co-glycolide) (PLGA), and other polyhydroxyacids, poly(caprolactone), polycarbonates, polyamides, polyanhydrides, polyphosphazene, polyamino acids, polyortho esters, polyacetals, polycyanoacrylates and biodegradable polyurethanes; non-biodegradable polymers such as polyacrylates, ethylene-vinyl acetate polymers and other acyl-substituted cellulose acetates and derivatives thereof; polyurethanes, polystyrenes, polyvinyl chloride, polyvinyl fluoride, poly(vinyl imidazole), chlorosulphonated polyolefins, and polyethylene oxide. Examples of biodegradable natural polymers include proteins such as albumin, collagen, fibrin, silk, synthetic polyamino acids and prolamines; polysaccharides such as alginate, heparin; and other naturally occurring biodegradable polymers of sugar units. Alternately, combinations of the aforementioned polymers can be used. In one aspect, a natural polymer that is not generally found in the extracellular matrix can be used.

PLA, PGA and PLA/PGA copolymers are particularly useful for forming biodegradable scaffolds. PLA polymers are usually prepared from the cyclic esters of lactic acids. Both L(+) and D(−) forms of lactic acid can be used to prepare the PLA polymers, as well as the optically inactive DL-lactic acid mixture of D(−) and L(+) lactic acids. Methods of preparing polylactides are well documented in the patent literature. The following U.S. Patents, the teachings of which are hereby incorporated by reference, describe in detail suitable polylactides, their properties and their preparation: U.S. Pat. No. 1,995,970 to Dorough; U.S. Pat. No. 2,703,316 to Schneider; U.S. Pat. No. 2,758,987 to Salzberg; U.S. Pat. No. 2,951,828 to Zeile; U.S. Pat. No. 2,676,945 to Higgins; and U.S. Pat. Nos. 2,683,136; 3,531,561 to Trehu.

PGA is a homopolymer of glycolic acid (hydroxyacetic acid). In the conversion of glycolic acid to poly(glycolic acid), glycolic acid is initially reacted with itself to form the cyclic ester glycolide, which in the presence of heat and a catalyst is converted to a high molecular weight linear-chain polymer. PGA polymers and their properties are described in more detail in Cyanamid Research Develops World's First Synthetic Absorbable Suture”, Chemistry and Industry, 905 (1970).

Fibers can be formed by melt-spinning, extrusion, casting, or other techniques well known in the polymer processing area. Preferred solvents, if used to remove a scaffold prior to implantation, are those which are completely removed by the processing or which are biocompatible in the amounts remaining after processing.

Polymers for use in the matrix should meet the mechanical and biochemical parameters necessary to provide adequate support for the cells with subsequent growth and proliferation. The polymers can be characterized with respect to mechanical properties such as tensile strength using an Instron tester, for polymer molecular weight by gel permeation chromatography (GPC), glass transition temperature by differential scanning calorimetry (DSC) and bond structure by infrared (IR) spectroscopy.

The substrate or scaffold can be nanopatterned or micropatterned with grooves an ridges that permit growth of cardiac tissues on the scaffold. Scaffolds can be of any desired shape and can comprise a wide range of geometries that are useful for the methods described herein. A non-limiting list of shapes includes, for example, patches, hollow particles, tubes, sheets, cylinders, spheres, and fibers, among others. The shape or size of the scaffold should not substantially impede cell growth, cell differentiation, cell proliferation or any other cellular process, nor should the scaffold induce cell death via e.g., apoptosis or necrosis. In addition, care should be taken to ensure that the scaffold shape permits appropriate surface area for delivery of nutrients from the surrounding medium to cells in the population, such that cell viability is not impaired. The scaffold porosity can also be varied as desired by one of skill in the art.

In some embodiments, attachment of the cells to a polymer is enhanced by coating the polymers with compounds such as basement membrane components, fibronectin, agar, agarose, gelatin, gum arabic, collagens types I, II, III, IV, and V, laminin, glycosaminoglycans, polyvinyl alcohol, mixtures thereof, and other hydrophilic and peptide attachment materials known to those skilled in the art of cell culture or tissue engineering. Examples of a material for coating a polymeric scaffold include polyvinyl alcohol and collagen. As will be appreciated by one of skill in the art, Matrigel™ is not suitable for administration to a human subject, thus the compositions described herein do not include Matrigel™.

In some embodiments it can be desirable to add bioactive molecules/factors to the scaffold. A variety of bioactive molecules can be delivered using the matrices described herein.

In one embodiment, the bioactive factors include growth factors. Examples of growth factors include platelet derived growth factor (PDGF), transforming growth factor alpha or beta (TGFβ), bone morphogenic protein 4 (BMP4), acidic fibroblast growth factor (aFGF), basis fibroblast growth factor (bFGF), fibroblastic growth factor 7 (FGF7), fibroblast growth factor 10 (FGF10), epidermal growth factor (EGF/TGFα), vascular endothelial growth factor (VEGF), nerve growth factor (NGF) some of which are also angiogenic factors.

These factors are known to those skilled in the art and are available commercially or described in the literature. Bioactive molecules can be incorporated into the matrix and released over time by diffusion and/or degradation of the matrix, or they can be suspended with the cell suspension.

Treatment of Cardiac or Neurodegenerative Disease and/or Injury

In some aspects, provided herein are methods for the treatment and/or prevention of a cardiac injury or a cardiac disease or disorder in a subject in need thereof. In some aspects, provided herein are methods for the treatment or prevention of a neurological disease or disorder. The methods described herein can be used to treat, ameliorate, prevent or slow the progression of a number of diseases or their symptoms, such as those resulting in pathological damage to the structure and/or function of the heart, brain, or spinal cord.

In some embodiments of any of the aspects, the method comprises transplanting a composition comprising cells treated to promote or enhance maturity as described herein into a subject.

The terms “cardiac disease,” “cardiac disorder,” and “cardiac injury,” are used interchangeably herein and refer to a condition and/or disorder relating to the heart. Such cardiac diseases or cardiac-related disease include, but are not limited to, myocardial infarction, heart failure, cardiomyopathy, congenital heart defect (e.g., non-compaction cardiomyopathy), hypertrophic cardiomyopathy, dilated cardiomyopathy, myocarditis, heart failure, arrhythmogenic right ventricular dysplasia (ARVD), cardiac arrhythmia, cardiomyopathy, long QT syndrome, catecholaminergic polymorphic ventricular tachycardia (CPVT), Barth syndrome, Duchenne muscular dystrophy-related cardiac disease, and cardiomegaly.

As used herein, the term, “neurological disease” refers to a disease that affects the central or peripheral nervous system of a subject. Non-limiting examples of neurological diseases includes Alzheimer's disease, Parkinson's disease, Down syndrome, dementia, multiple sclerosis, and amyotrophic lateral sclerosis (ALS).

As used herein, the terms “administering,” “introducing” and “transplanting” are used interchangeably in the context of the placement of cells, e.g. cardiomyocytes or neurons, as described herein into a subject, by a method or route which results in at least partial localization of the introduced cells at a desired site, such as a site of injury or repair, such that a desired effect(s) is produced. The cardiomyocytes can be implanted directly to the heart or brain, for example, or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years or more, i.e., long-term engraftment. As one of skill in the art will appreciate, long-term engraftment is desired as both cardiomyocytes and neurons generally do not proliferate to an extent that the heart or nervous tissues can heal from an acute injury comprising cardiomyocyte or neuronal cell death.

When provided prophylactically, the cardiomyocytes or neurons can be administered to a subject in advance of any symptom of a disorder, e.g., heart failure due to prior myocardial infarction or left ventricular insufficiency, congestive heart failure etc. Accordingly, the prophylactic administration of a population of cells serves to prevent a cardiac heart failure disorder or maladaptive cardiac remodeling, or, for example, symptoms of a neurodegenerative disorder.

In some embodiments of the aspects described herein, the population of cells being administered according to the methods described herein comprises allogeneic cells or their progeny obtained or derived from one or more donors. In this context, “allogeneic” refers to a cardiomyocytes or neurons differentiated in vitro from stem cells derived from one or more different donors of the same species, where the genes at one or more loci are not identical. For example, cardiomyocytes or neurons being administered to a subject can be derived from umbilical cord blood obtained from one more unrelated donor subjects, or from one or more non-identical siblings. In some embodiments, syngeneic cell populations can be used, such as those obtained from genetically identical animals, or from identical twins. In other embodiments of this aspect, the cardiomyocytes or neurons are autologous cells; that is, the cells are differentiated in vitro from stem cells, e.g., iPS cells, derived from a subject and administered to the same subject, i.e., the donor and recipient are the same.

### Administration and Efficacy

In one aspect, described herein is a method of transplanting in vitro-differentiated cardiomyocytes in a subject, the method comprising: (a) contacting in vitro-differentiated cardiomyocytes with an activator of AMPK; and (b) transplanting the in vitro-differentiated cardiomyocytes into the subject.

In another aspect, described herein is a method of transplanting in vitro-differentiated neurons in a subject, the method comprises: (a) contacting in vitro-differentiated neurons with an activator of AMPK; and (b) transplanting the in vitro-differentiated neurons into the subject.

In another aspect, described herein is a composition comprising in vitro-differentiated cardiomyocytes or in vitro-differentiated neurons that have been contacted with an activator of AMPK for use in a method of transplant.

While it is generally considered that AMPK activator treatment is performed on in vitro differentiated cells in vitro, prior to transplant, it is contemplated that AMPK activator treatment can be conducted or continued in vivo, for example, as a component of the cell formulation or composition (including, but not necessarily limited to a matrix or scaffold) administered to the subject, or separately. Local administration of AMPK activator at the site of implantation is specifically contemplated, and can include, for example, implantation of a depot, osmotic pump, or other device or formulation for local delivery or extended release of an AMPK activator. In other embodiments, however, the AMPK activator treatment is only performed in vitro, prior to transplant of the cells.

Provided herein are methods for treating a cardiac disease, a cardiac disorder, a cardiac injury, a neurological disease, or a neurological injury comprising administering cardiomyocytes or neurons to a subject in need thereof. In some embodiments, methods and compositions are provided herein for the prevention of an anticipated disorder e.g., heart failure following myocardial injury or Alzheimer's disease.

Measured or measurable parameters for efficacy include clinically detectable markers of function or disease, for example, elevated or depressed levels of a clinical or biological marker, functional parameters, as well as parameters related to a clinically accepted scale of symptoms or markers for health or a disease or disorder. It will be understood, however, that the total usage of the compositions and formulations as disclosed herein will be decided by the attending physician within the scope of sound medical judgment. The exact amount required will vary depending on factors such as the type of disease being treated.

The term “effective amount” as used herein refers to the amount of a population of cardiomyocytes and/or neurons needed to alleviate at least one or more symptoms of a disease or disorder, including but not limited to an injury, disease, or disorder. An “effective amount” relates to a sufficient amount of a composition to provide the desired effect, e.g., treat a subject having an infarct zone following myocardial infarction, improve cardiomyocyte engraftment, prevent onset of heart failure following cardiac injury, enhance vascularization of a graft, prevent or inhibit memory loss, etc. The term “therapeutically effective amount” therefore refers to an amount of human cardiomyocytes and/or neurons or a composition including such cells that is sufficient to promote a particular effect when administered to a typical subject, such as one who has, or is at risk for, a cardiac disease or neurological disorder. An effective amount as used herein also includes an amount sufficient to prevent or delay the development of a symptom of the disease, alter the course of a disease symptom (for example but not limited to, slow the progression of a symptom of the disease), or reverse a symptom of the disease. It is understood that for any given case, an appropriate “effective amount” can be determined by one of ordinary skill in the art using routine experimentation.

In some embodiments, the subject is first diagnosed as having a disease or disorder affecting the myocardium, brain or nervous tissue prior to administering the cells according to the methods described herein. In some embodiments, the subject is first diagnosed as being at risk of developing a disease (e.g., heart failure following myocardial injury or Alzheimer's disease) or disorder prior to administering the cells.

For use in the various aspects described herein, an effective amount of human cardiomyocytes and/or neurons comprises at least 1×10³, at least 1×10⁴, at least 1×10⁵, at least 5×10⁵, at least 1×10⁶, at least 2×10⁶, at least 3×10⁶, at least 4×10⁶, at least 5×10⁶, at least 6×10⁶, at least 7×10⁶, at least 8×10⁶, at least 9×10⁶, at least 1×10⁷, at least 1.1×10⁷, at least 1.2×10⁷, at least 1.3×10⁷, at least 1.4×10⁷, at least 1.5×10⁷, at least 1.6×10⁷, at least 1.7×10⁷, at least 1.8×10⁷, at least 1.9×10⁷, at least 2×10⁷, at least 3×10⁷, at least 4×10⁷, at least 5×10⁷, at least 6×10⁷, at least 7×10⁷, at least 8×10⁷, at least 9×10⁷, at least 1×10⁸, at least 2×10⁸, at least 5×10⁸, at least 7×10⁸, at least 1×10⁹, at least 2×10⁹, at least 3×10⁹, at least 4×10⁹, at least 5×10⁹ or more cardiomyocytes and/or neurons.

In some embodiments, a composition comprising cardiomyocytes treated with an AMPK activator permits engraftment of the cells in the heart at an efficiency at least 20% greater than the engraftment when such cardiomyocytes are administered without AMPK activator treatment; in other embodiments, such efficiency is at least 30/a, at least 40%, at least 50%, at least 60%, at least 70%, at least 80%, at least 90%, at least 1-fold, at least 2-fold, at least 5-fold, at least 10-fold, at least 100-fold or more than the efficiency of engraftment when cardiomyocytes are administered without treatment with an AMPK activator.

In some embodiments, an effective amount of cardiomyocytes is administered to a subject by intracardiac administration or delivery. In this context, “intracardiac” administration or delivery refers to all routes of administration whereby a population of cardiomyocytes is administered in a way that results in direct contact of these cells with the myocardium of a subject, including, but not limited to, direct cardiac injection, intra-myocardial injection(s), intra-infarct zone injection, injection during surgery (e.g., cardiac bypass surgery, during implantation of a cardiac mini-pump or a pacemaker, etc.). In some such embodiments, the cells are injected into the myocardium (e.g., cardiomyocytes), or into the cavity of the atria and/or ventricles. In some embodiments, intracardiac delivery of cells includes administration methods whereby cells are administered, for example as a cell suspension, to a subject undergoing surgery via a single injection or multiple “mini” injections into the desired region of the heart.

In some embodiments, a composition comprising neurons treated with an AMPK activator permits engraftment of the cells in the brain, spinal cord or nervous tissue at an efficiency at least 20% greater than the engraftment when such neurons are administered without AMPK activator treatment; in other embodiments, such efficiency is at least 30%, at least 40%, at least 50%, at least 60%, at least 70%, at least 80%, at least 90%, at least 1-fold, at least 2-fold, at least 5-fold, at least 10-fold, at least 100-fold or more than the efficiency of engraftment when neurons are administered alone without being treated with an AMPK activator.

In some embodiments, an effective amount of cardiomyocytes or neurons is administered to a subject by systemic administration, such as intravenous administration. In some embodiments, the cardiomyocytes or neurons are administered by a minimally invasive procedure, e.g., via a catheter or a port to the desired site of engraftment. The phrases “systemic administration,” “administered systemically”, “peripheral administration” and “administered peripherally” are used herein refer to the administration of a population of cardiomyocytes and/or neurons other than directly into a target site, tissue, or organ, such as the heart, such that it enters, instead, the subject's circulatory system.

The choice of formulation will depend upon the specific composition used and the number of cardiomyocytes and/or neurons to be administered; such formulations can be adjusted by the skilled practitioner. However, as an example, where the composition is cardiomyocytes and/or neurons in a pharmaceutically acceptable carrier, the composition can be a suspension of the cells in an appropriate buffer (e.g., saline buffer) at an effective concentration of cells per mL of solution. The formulation can also include cell nutrients, a simple sugar (e.g., for osmotic pressure regulation) or other components to maintain the viability of the cells. Alternatively, the formulation can comprise a scaffold, such as a biodegradable scaffold as described herein or as known in the art.

In some embodiments, additional agents to aid in treatment of the subject can be administered before or following treatment with the cardiomyocytes and/or neurons as described. Such additional agents can be used, for example, to prepare the target tissue for administration of the progenitor cells. Alternatively, the additional agents can be administered after the cardiomyocytes and/or neurons to support the engraftment and growth or integration of the administered cell into the heart, spinal cord, brain, or other desired administration site. In some embodiments, the additional agent comprises growth factors, such as VEGF, PDGF, FGF, aFGF, bFGF or NGF. Other exemplary agents can be used to reduce the load on the heart while the cardiomyocytes are engrafting (e.g., beta blockers, medications to lower blood pressure etc.).

The efficacy of treatment can be determined by the skilled clinician. However, a treatment is considered “effective treatment,” as the term is used herein, if any one or all of the symptoms, or other clinically accepted symptoms or markers of disease, e.g., cardiac disease, heart failure, cardiac injury and/or a cardiac disorder are reduced, e.g., by at least 10% following treatment with a composition comprising human cardiomyocytes cells as described herein. Methods of measuring these indicators are known to those of skill in the art and/or described herein.

Indicators of a cardiac disease or cardiac disorder, or cardiac injury include functional indicators or parameters, e.g., stroke volume, heart rate, left ventricular ejection fraction, heart rhythm, blood pressure, heart volume, regurgitation, etc. as well as biochemical indicators, such as a decrease in markers of cardiac injury, such as serum lactate dehydrogenase, or serum troponin, among others. As one example, myocardial ischemia and reperfusion are associated with reduced cardiac function. Subjects that have suffered an ischemic cardiac event and/or that have received reperfusion therapy have reduced cardiac function when compared to that before ischemia and/or reperfusion. Measures of cardiac function include, for example, ejection fraction and fractional shortening. Ejection fraction is the fraction of blood pumped out of a ventricle with each heartbeat. The term ejection fraction applies to both the right and left ventricles. LVEF refers to the left ventricular ejection fraction (LVEF). Fractional shortening refers to the difference between end-diastolic and end-systolic dimensions divided by end-diastolic dimension.

Non-limiting examples of clinical tests that can be used to assess cardiac functional parameters include echocardiography (with or without Doppler flow imaging), electrocardiogram (EKG), exercise stress test, Holter monitoring, or measurement of β-natriuretic peptide.

Indicators of a neurological disease or neurological disorder, or brain injury include functional indicators or parameters, e.g., memory, cognitive function, sensory or motor function, breathing, etc. as well as biochemical indicators, such as a decrease in markers of brain injury or disease, such as a reduction in N-acetylaspartate (NAA) or NAA to creatine ratio (NAA/Cr), amyloid beta (Aβ), or an increase in GABA to creatine ratio (GABA/Cr) and/or glutamate to creatine ratio (Glu/Cr), among others.

Where necessary or desired, animal models of injury or disease can be used to gauge the effectiveness of a particular composition as described herein. For example, an isolated working rabbit or rat heart model, or a coronary ligation model in either canines or porcines can be used. Animal models of cardiac function are useful for monitoring infarct zones, coronary perfusion, electrical conduction, left ventricular end diastolic pressure, left ventricular ejection fraction, heart rate, blood pressure, degree of hypertrophy, diastolic relaxation function, cardiac output, heart rate variability, and ventricular wall thickness, etc. Animal models for neurodegenerative diseases are also useful for determining the progression of symptoms with and without administration of compositions comprising neurons as described herein.

In some embodiments, a composition comprising cardiomyocytes as described herein is delivered at least 6 hours following the initiation of reperfusion, for example, following a myocardial infarction. During an ischemic insult and subsequent reperfusion, the microenvironment of the heart or that of the infarcted zone can be too “hostile” to permit engraftment of cardiomyocytes administered to the heart. Thus, in some embodiments it is preferable to administer such compositions at least 6 hours, at least 12 hours, at least 18 hours, at least 24 hours, at least 36 hours, at least 48 hours, at least 60 hours, at least 72 hours, at least 84 hours, at least 96 hours, at least 5 days, at least 6 days, at least 7 days, at least 8 days, at least 9 days, at least 10 days or more following the initiation of reperfusion. In some embodiments, the compositions comprising cardiomyocytes as described herein can be administered to an infarcted zone, peri-infarcted zone, ischemic zone, penumbra, or the border zone of the heart at any length of time after a myocardial infarction (e.g., at least 1 month, at least 6 months, at least one year, at least 2 years, at least 5 years, at least 10 years, at least 20 years, at least 30 years or more), however as will be appreciated by those of skill in the art, the success of engraftment following a lengthy interval of time after infarct will depend on a number of factors, including but not limited to, amount of scar tissue deposition, density of scar tissue, size of the infarcted zone, degree of vascularization surrounding the infarcted zone, etc. As such, earlier intervention by administration of compositions comprising cardiomyocytes may be more efficacious than administration after e.g., a month or more after infarct.

Compositions comprising cardiomyocytes as described herein can be administered to any desired region of the heart including, but not limited to, an infarcted zone, peri-infarcted zone, ischemic zone, penumbra, the border zone, areas of wall thinning, areas of non-compaction, or in area(s) at risk of maladaptive cardiac remodeling. Compositions comprising neurons as described herein can be administered to any desired region of the brain, spinal cord or nervous tissue. Preferably, the neurons described herein are administered to a site of injury or a diseased region of the brain. The site can be determined by a skilled physician using standard techniques in medical imaging.

### Screening Assays

Compositions comprising cardiomyocytes and/or neurons as described herein can be used in screening assays for determining the toxicity, or alternatively the efficacy of a bioactive agent on viability, maturation, electroconductivity etc. of a given cell type. A screening assay will contact stem cell-derived in vitro differentiated cardiomyocytes, neurons, or other cells matured as described herein via AMPK activator with a candidate agent, and one or more parameters of the cells will be monitored or measured in the presence and absence, and/or presence of various concentrations of the candidate agent. Combinations of two or more agents can be used if so desired. In one approach, an agent is screened in hopes that it does affect the cultured cell, e.g. to identify a drug that has a therapeutic effect on a target cell. In another approach, agents, including but not limited to agents that are therapeutically active on another cell type, can be screened for potential deleterious or detrimental effects, including but not limited to toxicity, against cardiomyocytes, neurons, or other cells matured as described herein.

As used herein, the term “test compound” or “candidate agent” refers to an agent or collection of agents (e.g., compounds) that are to be screened for their ability to have an effect on the cell. Test compounds can include a wide variety of different compounds, including chemical compounds, mixtures of chemical compounds, e.g., polysaccharides, small organic or inorganic molecules (e.g. molecules having a molecular weight less than 2000 Daltons, less than 1000 Daltons, less than 1500 Dalton, less than 1000 Daltons, or less than 500 Daltons), biological macromolecules, e.g., peptides, proteins, peptide analogs, and analogs and derivatives thereof, peptidomimetics, nucleic acids, nucleic acid analogs and derivatives, an extract made from biological materials such as bacteria, plants, fungi, or animal cells or tissues, naturally occurring or synthetic compositions.

Depending upon the particular embodiment being practiced, the test compounds can be provided free in solution, or can be attached to a carrier, or a solid support, e.g., beads. A number of suitable solid supports can be employed for immobilization of the test compounds. Examples of suitable solid supports include agarose, cellulose, dextran (commercially available as, i.e., Sephadex™, Sepharose™) carboxymethyl cellulose, polystyrene, polyethylene glycol (PEG), filter paper, nitrocellulose, ion exchange resins, plastic films, polyaminemethylvinylether maleic acid copolymer, glass beads, amino acid copolymer, ethylene-maleic acid copolymer, nylon, silk, etc. Additionally, for the methods described herein, test compounds can be screened individually, or in groups. Group screening is particularly useful where hit rates for effective test compounds are expected to be low such that one would not expect more than one positive result for a given group.

A number of small molecule libraries are commercially available. These small molecule libraries can be screened using the screening methods described herein. A chemical library or compound library is a collection of stored chemicals that can be used in conjunction with the methods described herein to screen candidate agents for a particular effect. A chemical library also comprises information regarding the chemical structure, purity, quantity, and physiochemical characteristics of each compound. Compound libraries can be obtained commercially, for example, from Enzo Life Sciences, Aurora Fine Chemicals, Exclusive Chemistry Ltd., ChemDiv, ChemBridge, TimTec Inc., AsisChem, and Princeton Biomolecular Research, among others.

Without limitation, the compounds can be tested at any concentration that can exert an effect on the cells relative to a control over an appropriate time period. In some embodiments, compounds are tested at concentrations in the range of about 0.01 nM to about 100 mM, about 0.1 nM to about 500 μM, about 0.1 μM to about 20 μM, about 0.1 μM to about 10 μM, or about 0.1 μM to about 5 μM.

The compound screening assay can be used in a high through-put screen. High through-put screening is a process in which libraries of compounds are tested for a given activity. High through-put screening seeks to screen large numbers of compounds rapidly and in parallel. For example, using microtiter plates and automated assay equipment, a laboratory can perform as many as 100,000 assays per day in parallel.

The compound screening assays described herein can involve more than one measurement of the cell or reporter function (e.g., measurement of more than one parameter and/or measurement of one or more parameters at multiple points over the course of the assay). Multiple measurements can allow for following the biological activity over incubation time with the test compound. In one embodiment, the reporter function is measured at a plurality of times to allow monitoring of the effects of the test compound at different incubation times.

The screening assay can be followed by a subsequent assay to further identify whether the identified test compound has properties desirable for the intended use. For example, the screening assay can be followed by a second assay selected from the group consisting of measurement of any of: bioavailability, toxicity, or pharmacokinetics, but is not limited to these methods.

Toxicity of an agent or test compound is indicated by the agent's effect on one or more of: cell viability, cell size, a biopotential or electrical property, mitochondrial function, gene expression, beat rate, and contractile function.

The screening assay can also determine the electrical, metabolic, contractile or other function of in vitro-differentiated cardiomyocytes in the presence of a test compound, in comparison to a reference level. With regard to neurons, the effect of a test compound on electrical, metabolic, or other function of in vitro-differentiated neurons can be measured as compared to a reference level. A change in any of these functions can indicate likely effects of the test compound or agent on the function or health of cardiac or neuronal cells or tissues.

Some embodiments of the compositions and methods described herein can be defined according to any of the following numbered paragraphs:
1. A method of promoting maturation of in vitro-differentiated cardiomyocytes, the method comprising treating in vitro-differentiated cardiomyocytes with an activator of adenosine monophosphate-activated protein kinase (AMPK).
2. The method of paragraph 1, wherein the activator of AMPK comprises a small molecule, a polypeptide, a nucleic acid encoding a polypeptide or a vector encoding a polypeptide.
3. The method of paragraph 2, wherein the small molecule is 5-aminoimidizole-4-carboxamide riboside (AICAR) or a derivative thereof that activates AMPK.
4. The method of paragraph 3, wherein the derivative is 5-aminoimidazole-4-carboxamide-1-β-D-ribofuranosyl-5′-monophosphate (ZMP).
5. The method of paragraph 2, wherein the polypeptide comprises AMPK.
6. The method of paragraph 1 or paragraph 2, wherein the activator comprises a vector encoding an AMPK polypeptide.
7. The method of paragraphs 2, 5 or 6, wherein the AMPK polypeptide is a constitutively active polypeptide.
8. The method of either of paragraphs 2 and 6, wherein the nucleic acid encoding the polypeptide or the vector that encodes the polypeptide permits inducible expression of the polypeptide.
9. The method of paragraph 2 or paragraph 6, wherein the vector is selected from the group consisting of: a lentiviral vector, an adenoviral vector, an adeno-associated virus vector (AAV), episomal vector, an EBNA1 vector, a minicircle vector, and a Sendai virus vector.
10. The method of any one of paragraphs 1-9, wherein the in vitro differentiated cardiomyocytes are human.
11. The method of any one of paragraphs 1-10, wherein the in vitro differentiated cardiomyocytes are differentiated from induced pluripotent stem cells (iPSCs) or from embryonic stem cells.
12. The method of any one of paragraphs 1-11, wherein the in vitro differentiated cardiomyocytes are derived from a subject having a cardiac disease or disorder.
13. The method of paragraph 12, wherein the cardiac disease or disorder is selected from the group consisting of: arrhythmogenic right ventricular dysplasia (ARVD), cardiomyopathy, cardiac arrhythmia, cardiomyopathy, long QT syndrome, catecholaminergic polymorphic ventricular tachycardia (CPVT), Barth syndrome, and Duchenne muscular dystrophy-related cardiac disease.
14. The method of any one of paragraphs 1-13, wherein treatment with an activator of AMPK promotes one or more of electrical maturity, metabolic maturity, and/or contractile maturity of in vitro-differentiated cardiomyocytes.
15. The method of paragraph 14, wherein electrical maturity is determined by one or more of the following markers as compared to a reference level: increased gene expression of an ion channel gene, increased sodium current density, increased inwardly-rectifying potassium channel current density, decreased action potential frequency, decreased calcium wave frequency, and decreased field potential frequency.
16. The method of paragraph 14, wherein metabolic maturity of in vitro-differentiated cardiomyocytes is determined by one or more of the following markers as compared to a reference level: increased activity of mitochondrial function, increased fatty acid metabolism, increased oxygen consumption rate (OCR), increased phosphorylated ACC levels or activity, increased level or activity of fatty acid binding protein (FABP), increased level or activity of pyruvate dehydrogenase kinase-4 (PDK4), increased mitochondrial respiratory capacity, increased mitochondrial volume, and increased levels of mitochondrial DNA.
17. The method of paragraph 14, wherein contractile maturity is determined by one or more of the following markers as compared to a reference level: decreased beat frequency, increased contractile force, increased level or activity of α-myosin heavy chain (α-MHC), increased level or activity of sarcomeres, decreased circularity index, increased level or activity of troponin, increased level or activity of titin N2b, increased cell area, and increased aspect ratio.
18. The method of any one of paragraphs 1-17, further comprising contacting the in vitro-differentiated cardiomyocytes with a nanopatterned substrate.
19. A method of transplanting in vitro-differentiated cardiomyocytes in a subject, the method comprising:
   (a) contacting in vitro-differentiated cardiomyocytes with an activator of AMPK; and
   (b) transplanting said in vitro-differentiated cardiomyocytes into the subject.
20. The method of paragraph 19, further comprising administering metformin to the subject.
21. The method of paragraph 20, wherein the metformin modulates the electrical maturity, metabolic maturity, and/or contractile maturity of in vitro-differentiated cardiomyocytes.
22. The method of paragraph 20 or paragraph 21, wherein the metformin enhances engraftment of the in vitro-differentiated cardiomyocytes.
23. A method of promoting maturation of in vitro-differentiated neurons, the method comprising contacting in vitro-differentiated neurons with an activator of adenosine monophosphate-activated protein kinase (AMPK).
24. The method of paragraph 23, wherein the activator of AMPK comprises a small molecule, a polypeptide, a nucleic acid encoding a polypeptide or a vector encoding a polypeptide.
25. The method of paragraph 24, wherein the small molecule is 5-aminoimidizole-4-carboxamide riboside (AICAR) or a derivative thereof that activates AMPK.
26. The method of paragraph 25, wherein the derivative is 5-aminoimidazole-4-carboxamide-1-β-D-ribofuranosyl-5′-monophosphate (ZMP).
27. The method of paragraph 24, wherein the polypeptide comprises AMPK.
28. The method of paragraph 23 or paragraph 24, wherein the activator comprises a vector encoding an AMPK polypeptide.
29. The method of any one of paragraphs 24, 27 or 28, wherein the AMPK polypeptide is a constitutively active polypeptide.
30. The method of either of paragraphs 24 or paragraph 28, wherein the nucleic acid encoding the polypeptide or the vector that encodes the polypeptide permits inducible expression of the polypeptide.
31. The method of paragraph 24 or paragraph 28, wherein the vector is selected from the group consisting of: a lentiviral vector, an adenoviral vector, an adeno-associated virus vector (AAV), episomal vector, an EBNA1 vector, a minicircle vector, and a Sendai virus vector.
32. The method of any one of paragraphs 23-31, wherein the in vitro differentiated neurons are human.
33. The method of any one of paragraphs 23-32, wherein the in vitro differentiated neurons are differentiated from induced pluripotent stem cells (iPSCs) or from embryonic stem cells.
34. The method of any one of paragraphs 23-33, wherein the in vitro differentiated neurons are derived from a subject having a neurological disease or disorder.
35. The method of paragraph 34, wherein the neurological disease or disorder is selected from the group consisting of: Alzheimer's disease, Parkinson's disease, Down syndrome, dementia, multiple sclerosis, and amyotrophic lateral sclerosis (ALS).
36. The method of any one of paragraphs 23-35, wherein treatment with an activator of AMPK promotes a reduction in the level or activity of amyloid beta (Aβ) or phosphorylated Tau protein.
37. The method of any one of paragraphs 23-36, wherein treatment with an activator of AMPK promotes electrical maturity or metabolic maturity of in vitro-differentiated neurons.
38. The method of any one of paragraphs 23-37, wherein treatment with an activator of AMPK promotes maturity of in vitro-differentiated neurons as compared to a reference level, in one or more of the following markers of maturity: increased levels or activity of PPARα, increased levels or activity of TFAM, increased levels or activity of PDK4, increased levels or activity of NeuN, reduced levels or activity of amyloid beta (Aβ), reduced levels or activity of phosphorylated Tau protein, increased activity of mitochondrial function, increased fatty acid metabolism, and increased levels of mitochondrial DNA.
39. The method of paragraph 36 or paragraph 38 wherein the Aβ is Aβ₁₋₄₂.
40. A method of evaluating toxicity of an agent, the method comprising contacting in vitro-differentiated cardiomyocytes or neurons prepared by the method of any one of paragraphs 1-39, respectively, with an agent.
41. The method of paragraph 40, further comprising detecting at least one phenotypic characteristic of the cardiomyocytes or neurons.
42. The method of paragraph 40 or paragraph 41, wherein the agent is selected from the group consisting of a small molecule, an antibody, a peptide, a genome editing system, and a nucleic acid.
43. The method of any one of paragraphs 40-42, wherein toxicity of an agent is indicated by the agent's effect on one or more of: cell viability, cell size, a biopotential or electrical property, mitochondrial function, gene expression, beat rate, and contractile function.
44. A composition comprising in vitro-differentiated cardiomyocytes made by contacting in vitro-differentiated cardiomyocytes with an activator of adenosine monophosphate-activated protein kinase (AMPK), wherein the cardiomyocytes have a more mature phenotype as compared with in vitro-differentiated cardiomyocytes that were not contacted with an activator of adenosine monophosphate-activated protein kinase (AMPK).
45. A composition comprising in vitro-differentiated neurons made by contacting in vitro-differentiated neurons with an activator of adenosine monophosphate-activated protein kinase (AMPK), wherein the neurons have a more mature phenotype as compared with in vitro-differentiated neurons that were not contacted with an activator of adenosine monophosphate-activated protein kinase (AMPK).
46. An activator of AMPK for use in promoting the maturation of in vitro-differentiated cardiomyocytes.
47. An activator of AMPK for use in promoting the maturation of in vitro-differentiated neurons.
48. A composition comprising in vitro-differentiated cardiomyocytes and an activator of AMPK for use in the treatment of a cardiac disease or disorder.
49. A composition comprising in vitro-differentiated neurons and an activator of AMPK for use in the treatment of a neurological disease or disorder.
50. A composition of paragraph 44 for use in the treatment of a cardiac disease or disorder.
51. A composition of paragraph 45 for use in the treatment of a neurological disease or disorder.
52. A composition of paragraph 44 for use in a transplant to cardiac tissue of a subject in need thereof.
53. A composition of paragraph 45 for use in a transplant to neuronal tissue of a subject in need thereof.

Unless otherwise explained, all technical and scientific terms used herein have the same meaning as commonly understood by one of ordinary skill in the art to which this disclosure belongs.

It should be understood that this disclosure is not limited to the particular methodology, protocols, and reagents, etc., described herein and as such may vary. The terminology used herein is for the purpose of describing particular embodiments only, and is not intended to limit the scope of the present disclosure, which is defined solely by the claims.

All patents and other publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents are based on the information available to the applicants and do not constitute any admission as to the correctness of the dates or contents of these documents.

### EXAMPLES

### Example 1: Activation of AMPK Promotes Maturation of Cardiomyocytes and Neurons Derived from Human Pluripotent Stem Cells

### Introduction

The immaturity of human pluripotent stem cell (hPSC) derivatives limits their utility for drug screening, disease modeling, and tissue repair. Adenosine monophosphate-activated protein kinase (AMPK) is a developmentally regulated energy sensor that controls many cellular pathways. It was tested whether activating AMPK would induce metabolic, structural, and functional maturation in hPSC-cardiomyocytes (CMs), -neurons, and -hepatocytes. Activating AMPK with the small molecule, AICAR, more than doubles the ability of hPSC-CMs to oxidize fatty acids, associated with increased phosphorylation of acetyl CoA carboxylase, mitochondrial maximum respiration capacity, and mitochondrial biogenesis. AMPK activation increases hPSC-CM size and elongation, induces protein isoform switching for titin and troponin I, and increases the contractile force and passive tension of the hPSC-CMs. AMPK activation phosphorylates multiple intracellular signaling kinases, including protein kinase B (Akt), extracellular signal regulated-kinase (ERK), and p38-mitogen-activated protein kinase (p38-MAPK). This accelerated maturation allowed detection of a disease phenotype in hiPSC-CMs from patients with adult onset arrhythmogenic cardiomyopathy. Overexpressing constitutively active AMPKα1 and α2 phenocopies AICAR treatment. AMPK also promotes the mitochondria biogenesis in hiPSC-neurons and enhances the expression of NeuN, a neuronal maturation marker. Conversely, in hiPS-derived hepatocytes, AMPK activation decreased maturation and did not promote mitochondria biogenesis. Thus, AMPK activation has context-dependent effects on maturation. Controlling maturation via AMPK may facilitate use of hPSC derivatives for therapeutic applications and disease modeling.

Promoting the maturation of hPSC-CMs is essential for their use in a broad range of applications such as cardiac regenerative therapies, disease modeling, and drug screening. A variety of approaches using two dimensional culture and three dimensional tissue engineering have been taken and significant progress has been achieved to enhance hPSC-CM maturation^{1,2,3}. It is now possible, by long-term culture^{3, 4, and others}, hormonal treatment⁵⁻⁷, substrate stiffness^{8, 9}, microRNAs^{10, 11}, to obtain hPSC-CMs exhibit some morphological and molecular characteristics similar to adult cardiomyocytes, and displaying better-developed calcium-handling apparatus and greater contractile force^{2,3, 12}. Generating tissue constructs that produce Frank-Starling curves has also been demonstrated¹³, and interventions such as mechanical conditioning have been shown to increase cardiac contractility at the tissue level¹⁴. However, little is known in regard to the metabolic aspects of hPSC-CM maturation. The immature cardiomyocytes rely heavily on glucose¹⁵ and approaches for enhancing the fatty acid oxidation capabilities of hPSC-CMs have not been well investigated. In fact, it is currently unclear whether hPSC-CMs are capable of oxidizing fatty acids at all.

AMPK is a ubiquitously expressed heterotrimeric kinase that is viewed as a “cellular fuel gauge” and “super metabolic regulator”¹⁶. It is a serine-threonine kinase that is allosterically activated by increases in the ratio of [AMP] or [ADP] to [ATP]. Its activation also requires phosphorylation of the α subunit, which can occur via liver kinase B1 (LKB1) or calcium/calmodulin-dependent protein kinase kinase 2 (CAMKKβ), although the LKB1 pathway is thought to predominate in cardiomyocytes¹⁶. Shortly after birth, cardiac AMPK protein and activity levels increase in association with the conversion from glucose metabolism to fatty acid oxidation¹⁷. AMPK directly regulates pathways involved in fatty acid and glucose transport into the cell, increases glycolytic flux, and enhances mitochondrial entry of fatty acyl carnitine¹⁷. Additionally, AMPK regulates transcription via the estrogen-related receptor-α (ERR-α)¹⁸ and peroxisome proliferator-activated receptor coactivator 1-α (PGC-1α)¹⁹. Through these pathways, AMPK contributes to control of mitochondrial biogenesis and other gene regulatory networks. Without being bound by a particular theory it was hypothesized that AMPK agonist treatment leads to cardiac metabolic maturation.

AICAR (5-amino-4-imidazolecarboxamide riboside-1-β-D-ribofuranoside) has been widely used to induce AMPK activation in cardiomyocytes^{20, 21}. AICAR is taken up by adenosine transporters and subsequently phosphorylated by adenosine kinase to ZMP (5-aminoimidazole-4-carboxamide-1-β-D-furanosyl 5′-monophosphate), which in turn mimics AMP to activate AMPK. In this study, it was found that AMPK activation by AICAR markedly enhances the fatty acid oxidation capacity of hPSC-CMs partly by phosphorylating ACC. Surprisingly, it was observed that AMPK activation leads to enhanced mitochondrial biogenesis, increase in cell size, a decrease in circularity index, enhanced cardiac gene expression, and improved contractile force production. Also, the effect of AMPK activation on the maturation of neurons, and hepatocytes-derived from hPSCs was examined.

### 2. Methods

2.1 Cell Culture

Undifferentiated human IMR90-induced pluripotent stem cells, originally derived from lung fibroblasts²² (James A. Thomson, University of Wisconsin-Madison), were expanded using mouse embryonic fibroblast-conditioned medium supplemented with 5 ng/ml basic fibroblast growth factor. Cardiomyocytes were obtained using a previously described protocol² that involves the serial application of activin A and bone morphogenetic protein-4 (BMP4) under serum-free, monolayer culture conditions. The cultures were also supplemented with the Wnt agonist CHIR99021 in the early stages of differentiation followed by the Wnt antagonist Xav939. In this study, cardiomyocytes derived from human undifferentiated IMR90-induced stem cells were used, and for brevity, these are referred to human induced pluripotent stem cell-derived cardiomyocytes (hiPSC-CM). After 20 days of in vitro differentiation, the cells were dispersed using 0.05% trypsin-EDTA and re-plated. Cultures were fed every other day thereafter with serum-free RPMI-B27 plus insulin. Only cell preparations containing >80% cardiac troponin T-positive cardiomyocytes (by flow cytometry) were used for the current investigation. Twenty days after induction, cells were re-plated at different densities based on experimental purposes (described below). Cells were treated with 1 mM AICAR for two weeks (unless otherwise mentioned specifically), with media being changed every other day. This study was mainly performed with cardiomyocytes-derived from IMR90 iPS cell line. However, cardiomyocytes-derived from another iPS cell line, WTC11 iPS cells²², and a human embryonic stem cell line (hESC), RUES2 cells, were also used for some of the experiments. Rues and WTC undifferentiated cells were cultured with mTeSR medium. Similar differentiation protocols as to IMR90 were used to obtain cardiomyocytes from Rues and WTC cells. Only cell preparations containing >80% cardiac troponin T-positive cardiomyocytes (by flow cytometry) were used for the current investigation. Undifferentiated iPSCs were maintained on StemAdhere™ (Primorigin Biosciences®) with mTesR medium supplemented with bFGF. The formation of hepatocyte-like cells from iPSCs was achieved as described previously^{24,25}. Briefly, cells were induced to form endoderm by addition of 100 ng/ml Activin A, 10 ng/ml BMP4, and 20 ng/ml FGF2 for two days followed by 100 ng/ml Activin A for an additional three days. Hepatic progenitor cells were formed by supplementing the medium with 20 ng/ml BMP4 and 10 ng/ml FGF2. Next, 20 ng/ml HGF was added between days 10 and 15 and induced maturation of the hepatocytes by culturing the cells in HCM medium (Lonza®) containing 20 ng/ml Oncostatin M until day 20. In some experiments, the differentiation medium was supplemented with 1.0 mM AICAR between days 15 and 20. hiPSC-derived neurons were generated following previously published protocols^{26, 27, 28}. Briefly, hiPSCs from a control individual²⁸ and from a patient with the duplication of the amyloid precursor protein (APP) gene²⁷ were cultured on mouse embryonic fibroblasts in the presence of 20 ng/ml bFGF (Peprotech®). Neural stem cells were generated by hiPSC co-culture on the mouse stromal cell line PA6 in the presence of 0.5 ug/ml Noggin (Peprotech®) and 10 uM SB431542 (Peprotech®). After 12 days, neural rosettes were dissociated and FACS sorted for the cell surface markers CD184/CD24⁺; CD44/CD271⁻. Neural stem cells were propagated in 20 ng/ml bFGF and neuronal differentiation was induced by the withdrawal of bFGF and the addition of 20 ng/ml BDNF (Peprotech®), 20 ng/ml GDNF (Peprotech®), 0.5 uM dbCAMP (Peprotech®).

Secreted Amyloid beta (Ab) peptides were measured from the neuronal culture media at one, two and three-week time points after AICAR treatment by ELISA assay (Meso Scale Discovery®).

2.2 Mitochondrial Oxidation of Palmitate

The Seahorse XF96 extracellular flux Analyzer™ was used to assess the fatty acid oxidation capacity of the hPSC-CMs. XF96 plates were pre-treated with fibronectin at a concentration of 1 μg/cm². At around 20 days after induction of differentiation, the cardiomyocytes were seeded onto the XF96 plates at a density of 30,000 cells per well (2,500/mm²). The cells were treated with 1 mM AICAR for two weeks before the palmitate-albumin assay. An hour prior to performing this assay, culture medium was exchanged for basal media (unbuffered DMEM, Sigma D5030, supplemented with 2 mM glutamine, 1 mM pyruvate, 25 mM glucose, and 400 μM carnitine). Palmitate-albumin complexes were made following the Seahorse Bioscience protocol. Substrates were injected during the measurements to achieve two spike concentrations of palmitate-albumin at 200 μM each. The oxygen consumption rates (OCR values) were further normalized to the number of cells present in each well, quantified by staining with Hoechst 33342 (Sigma-Aldrich) as measured using fluorescence at 355 nm excitation and 460 nm emission. The highest OCR values induced by palmitate-albumin were used for statistics. Due to variations in the absolute magnitude of OCR measurements in different experiments, the relative AICAR treated/untreated control levels were used to compare and summarize independent biological replicates (N=7).

2.3 Mitochondrial Oxidation of Glucose

The Seahorse XF96 extracellular flux Analyzer™ was used to assess mitochondria function with glucose as substrate. Cell preparation and treatment for this assay were the same as the palmitate assay. On the assay day, culture medium was changed to base media (unbuffered DMEM, Sigma D5030, supplemented with 2 mM glutamine, 25 mM glucose, 1 mM pyruvate) 1 hour before the assay and for the duration of the measurement. Selective inhibitors were injected during the measurements to achieve final concentrations of oligomycin at 2.5 μM, FCCP at 1 μM, rotenone at 2.5 μM, and antimycin A at 2.5 μM. The OCR values were further normalized to the number of cells present in each well, quantified by Hoechst staining. Maximal OCR was the OCR difference between uncoupler FCCP and ATPase inhibitor oligomycin. Due to variations in the absolute magnitude of OCR measurements in different experiments, the relative AICAR treated/untreated control levels were used to compare and summarize independent biological replicates (N=6).

2.4 Electron Microscopy and Point Counting

Adherent cells in tissue culture dishes were fixed for transmission electron microscopy in half-strength Karnovsky's fixative (2.0% paraformaldehyde, 2.5% glutaraldehyde, 0.1M phosphate buffer, pH 7.4) overnight. The cells were then washed in 0.1M phosphate buffer two times (5 minutes each) and post-fixed in 1.0% OsO4 in 0.1M phosphate buffer. Cells were rinsed twice (5 minutes each) then dehydrated in 50/6, 70% and 90% alcohol, 2 minutes each, then in 100% alcohol twice (5 minutes each). Cells were embedded in Polybed 812 Resin™ (Ted Pella®, Redding, Calif.) by first infiltrating with a mixture of 50% alcohol/50% polybed for 1 hour and then pure polybed for 3 hours. Beem capsules filled with fresh resin were inverted over areas of cells. Polymerization procedure was performed for 24 hours at 65° C. Removal of beem capsules was achieved by placing plates in boiling water then gently peeling the capsule containing the polymerized resin and cells away from the plastic plate. Sections for light and transmission electron microscopy were cut using a Reichert Ultracut E Microtome™. Sections were mounted on copper grids and stained with uranyl acetate and lead citrate. Samples were examined at an accelerating voltage of 80 kV using a Tecnai G2 Spirit BioTWIN™ transmission electron microscope (FEI®, Hillsboro, Or, USA). Images were acquired using a side-mounted digital camera (Advanced Microscopy Techniques Corp®., Woburn, Mass., USA). The relative mitochondrial volume was calculated by point counting, in which 20 horizontal and 20 perpendicular lines were drawn in the images and the crosspoints that overlapped with mitochondria was counted and then divided by the total points.

2.5 Western Blotting

Total protein was acquired from control cardiomyocytes or cardiomyocytes after two weeks of AICAR treatment and subjected to SDS-PAGE. The lanes were loaded with equal amount of protein and were checked by Ponceau S staining. After blocking with 5% milk, the membranes were incubated with anti-pACC (Ser 79) rabbit polyclonal antibody (Cell Signaling Technology), anti-pAMPK, anti-pAkt, anti-pERK, anti-p38-MAPK, or anti-GAPDH mouse monoclonal antibody (Abcam) overnight while shaking at 4° C. After incubation with anti-rabbit (for pACC) and anti-mouse (for GAPDH, pAMPK, pAkt, pERK, and p-p38-MAPK) horseradish peroxidase-coupled secondary antibody (Santa Cruz Biotechnology®), bands were visualized with SuperSignal West Femto Trial Kit™ (Thermo Scientific®). The immunoblots band densities were analyzed in Image J.

2.6 Quantitative RT-PCR

Total RNA was isolated using the Qiagen RNeasy Kit™, and mRNA was reverse transcribed using the Superscript™ III first strand cDNA synthesis kit (Invitrogen®). All primers were purchased from Real Time Primers, and qRT-PCR was performed using SYBR™ green chemistry and an ABI 7900HT instrument or TAQMAN™ with BIORad CXF384™. Samples were normalized using hypoxanthine-guanine phosphoribosylthransferase (HPRT) as a housekeeping gene. The sequences of the primers are listed below in Table 1. The primers used for titin isoforms were adapted from²⁹. All Q-RT-PCR assays were performed in triplicate on control and experimental groups and firstly normalized to hHPRT level and then normalized against control cardiomyocytes. N=4-5 independent experiments.

TABLE 1
PRIMERS FOR QPCR

| Transcript | Forward Primer Sequence | Reverse Primer Sequence |
|---|---|---|
| HPRT | TGACACTGGCAAAACAATGCA | GGTCCTTTTCACCAGCAAGCT |
| α-MHC | CAAGTTGGAAGACGAGTGCT | ATGGGCCTCTTGTAGAGCTT |
| KCNJ2 | CTTCGGAGAAGCCTAGGCGC | AGCGCTACAGCAGTGTGACA |
| SCN5a | AGCTCTGTCACGATTTGAGG | AGGACTCACACTGGCTCTTG |
| ERRα | CCTCTGTGACCTCTTTGACC | TACTGACATCTGGTCAGACA |
| PPARα | ATTACGGAGTCCACGCGTGTG | TTGTCATACACCAGCTTGAGT or |
| | or | TTCAGGTCCAAGTTTGCGAAGC |
| | CAGAACAAGGAGGCGGAGGTC | |
| TFAM | CGCTCCCCCTTCAGTTTTGT or | CCAACGCTGGGCAATTCTTC or |
| | CCGAGGTGGTTTTCATCTGT | GCATCTGGGTTCTGAGCTTT |
| PGClα | TCTGAGAGGGCCAAGCAAAG or | GTCCCTCAGTTCTGTCCGTG or |
| | GTCACCACCCAAATCCTTAT | ATCTACTGCCTGGAGACCTT |
| FABP | CCGATTGGCAGAGTAGTAG | CAGCAGATGACAGGAAGG |
| PDK4 | AGAGGTGGAGCATTTCTCGC | ATGTTGGCGAGTCTCACAGG |
| Total titin | GTAAAAAGAGCTGCCCCAGTG | GCTAGGTGGCCCAGTGCTACT |
| | A | |
| Titin N2BA | CAGCAGAACTCAGAATCGA | ATCAAAGGACACTTCACACTC |
| Titin N2B | CCAATGAGTATGGCAGTGTCA | TACGTTCCGGAAGTAATTTGC |
| ssTnI | CCCAGCTCCACGAGGACTGAAC | TTTGCGGGAGGCAGTGATCTTG |
| | A | G |
| cTnI | GGAACCTCGCCCTGCACCAG | GCGCGGTAGTTGGAGGAGCG |
| ND1 | GTCAACCTCGCTTCCCCACCCT | TCCTGCGAATAGGCTTCCGGCT |
| LPL | CGAGTCGTCTTTCTCCTGATGA | TTCTGGATTCCAATGCTTCGA |
| | T | |
| HNF4α | GGTGTCCATACGCATCCTTGAC | AGCCGCTTGATCTTCCCTGGAT |
| Rpl13a | CTCAAGGTGTTTGACGGCATCC | TACTTCCAGCCAACCTCGTGAG |
| NeuN | CCGAGTGATGACCAACAAGA | GAATTCAGGCCCGTAGACTG |

| Transcript | Primer1 | Primer2 | Probe |
|---|---|---|---|
| ALB | AAATCCCACTGCA | AGCAGCAGCACGACA | /56-FAM/TG CCT |
| | TTGCCGAAGTG | GAGTAATCA | GCT G/Zen/A CTT |
| | | | GCC TTC ATT AGC |
| | | | T/3IABkFQ/ |
| AFP | CTGCAATTGAGAA | TTCCCTCTTCACTTTG | TTGGAGAAGTACG |
| | ACCCACTG | GCTG | GACATTCAGACTG |
| | | | C |
| SLC10A1 | TGTACAGGAGGAG | ACCTGTCCAATGTCTT | AACCTCAGCATTG |
| | AGGCATC | CAGTC | TGATGACCACCT |
| HNF4 | CTCATAGCTTGAC | GGTGGACAAAGACAA | /56-FAM/tctggacgg |
| | CTTCGAGTG | GAGGAA | /ZEN/CTT |
| | | | CCttatcatgc/3IABkFQ/ |
| PXR | GCACCGGaTTGTTC | GTACAAAGTCAGCAT | AGTCCAAgA/ZEN/G |
| | AAAGTG | GGTTCC | GCcCAGAAgCAAA |
| ApoB | CATTGCCCTTCCTC | CCAGAGACAGAAGAA | /56-FAM/CT GGA |
| | GTCTT | GCCAAG | TAC C/Zen/G TGT |
| | | | ATG GAA ACT GCT |
| | | | CC/3IABkFQ/ |
| ApoH | GCCCATCAACACT | CGCCATTCAGATAAA | ACGGCTCCATTTTC |
| | CTGAAATG | ACCCAG | TAAGATTCCAGCA |
| ASGR | TCCTTTCTGAGCC | TGAAGTCGCTAGAGT | /56- |
| | ATTGCC | CCCAG | FAM/CGTGAAGCA/ |
| | | | ZEN/GTTCGTGTCT |
| | | | GACCT/3IABkFQ/ |
| | GCCTTCACAGCGT | CGAAGATGGCGGAGG | /56- |
| | ACGA | TG | FAM/AGCAGTACC/ |
| | | | ZEN/TGTTTAGCCA |
| | | | CGATGG/3IABkFQ/ |

Mitochondrial DNA (mtDNA) to nuclear DNA (nDNA) ratio was estimated by q-PCR. For this purpose, a mtDNA fragment within the NADH dehydrogenase 1 (ND 1) gene and a region of the nuclear DNA-encoded lipoprotein lipase gene (LPL) were amplified. The primer sequences for ND 1 and LPL were adapted from³⁰. Total DNA was extracted using Qiagen DNeasy Kit™.

2.7 Immunocytochemistry

Cells were fixed in 4% paraformaldehyde for 10 min followed by PBS wash. The fixed cells were blocked with 1.5% normal goat serum for 1 hour at room temperature and incubated overnight at 4° C. with primary antibodies. Mouse anti-α-actinin (Sigma) was used. The samples were rinsed with PBS and incubated with a secondary antibody. Samples subjected to F-actin staining were incubated with TRITC-labeled phalloidin (Sigma) for 5 min at room temperature. Nuclei were stained with Hoechst 33342.

2.8 Imaging and Morphological Analysis

Fluorescent images were acquired using a Zeiss AxioCam™ mounted on a Zeiss AxioObserver Microscope™, and confocal images were processed and quantified using NIS Elements. Each cell was analyzed for cell size and circularity index.

2.9 Contractile Force Assessment

Contractile force was assessed using micropost arrays, following a procedure similar to previously reported studies on cardiomyocytes^{2, 31, 32}. The process used to fabricate the micropost arrays is described in detail elsewhere^{33, 34}. The microposts (6.45 μm in height, 2.3 μm in diameter, 6 μm center-to-center post spacing) were cast onto 25 mm diameter round #1 glass coverslips (VWR®). To enable cell attachment, the tips of these microposts were stamped with 50 μg/ml of mouse laminin (Life Technologies®) via microcontact printing, while the remaining surfaces of the micropost array were fluorescently stained with BSA conjugates with Alexa Fluor 594 and blocked with 0.2% Pluronic F-127 (in PBS)³⁴. Twenty days following differentiation, hiPSC-derived cardiomyocytes were seeded onto the micropost arrays in Attoflour chambers at a density of approximately 500,000 cells per chamber (˜100,000 cells/cm²), in RPMI media supplemented with 5% fetal bovine serum. After 24 hours, the medium was replaced with serum-free RPMI-B27 plus insulin, which then was exchanged every other day. Beginning two days after seeding, half of the substrates were treated with 1 mM AICAR for one week. Prior to live imaging, the media was exchanged to Tyrode's buffer. Individual cardiomyocyte twitch forces were recorded under phase light using high-speed video microscopy in a live cell chamber at 37° C., as previously described³². Only the contractile forces of single cardiomyocytes (no junctions with adjacent cells) with obvious beating activity were assessed. Post deflections were optically measured at approximately 100 frames/sec on a Nikon Ti-E™ upright microscope with a 60× water immersion objective. A custom-written MATLAB™ code was used to compare each time frame of the video to a reference fluorescent image taken at the base of the posts. Contractile forces were subsequently calculated by multiplying the deflection of each microposts by its bending stiffness:

F=kδ (1)

where F is the force at a single micropost, k is the post's bending stiffness (44.54 nN/μm for this study), and δ is the horizontal distance between the centroid of the post's tip and the centroid of the post's base. The total contractile force for each cardiomyocyte was calculated as the sum of the forces at each post beneath it. The passive tension, or baseline force, was defined as the contractile force when a cardiomyocyte was at rest, i.e. in between beats. The total twitch force was defined as the difference between the peak contractile force (achieved during a twitch) and the passive tension. Additionally, the average twitch force per post was analyzed in order to normalize the total twitch force by cell area. The average twitch force per post was calculated as the total twitch force divided by the number of posts underneath a cardiomyocyte. Passive tension, total twitch force, average twitch force per post, and beating frequency were used to compare the independent biological replicates (N=2, with 16-38 cells per independent biological replicate).

3.0 Recombinant Adenoviruses

To confirm the findings with the pharmacological AMPK activator AICAR, the adenoviruses were obtained encoding constitutively active forms of both AMPK α1³⁵ and AMPK α2³⁶. Briefly, AMPKα2-CA was created by truncating a full-length rat AMPKα2 cDNA at residue 312 while cDNA encoding residues 1 to 312 of α1, containing a mutation that alters threonine 172 to an aspartic acid (T172D) was used to construct the recombinant adenovirus Ad-CA-AMPK α1. The viruses were amplified in HEK 293 cells and purified using the standard CsCl₂ protocol. Human PSC-CMs were transfected with 10 plaque-forming units/cell of Ad-GFP or Ad AMPK α1-CA and Ad AMPK α2-CA for 4 hours and the medium were switched back to normal medium.

3.1 Plakophilin 2 iPSC Culture, Cardiomyocyte Differentiation, and Cell Viability Assay

The normal and mutant Plakophilin 2 (PKP2) cells were generated as described previously³⁷. Induced pluripotent cells were cultured with E8 medium in Matrigel® coated plates. Cardiomyocytes were obtained using a protocol based on Lian et al³⁸ that involves Wnt agonist CHIR 99021 in the early stages of differentiation followed by the Wnt antagonist IWP2 under monolayer condition. Before Day 7, cells were cultured in RPMI plus B27 without insulin while on Day 7 and afterwards, EB media (KO-DMEM with 2% FBS, 1 mM NEAAs, 1× GlutaMAX, 1 mM mercaptoethanol, and penicillin/streptomycin) was used. At 30 days of differentiation, beating EBs were treated with three factors (3F) (50 μg/ml insulin, 0.5 μM dexamethasone, and 0.25 mM IBMX) or five factors (5F) (3F plus PPAR-γ agonists 5 μM rosiglitazone and 200 μM indomethacin) media as described earlier or treated with 1 mM AICAR or AICAR plus rosiglitazone and indomethacin for two weeks. TUNEL staining with In Situ Cell Death Detection Kit TMR Red™ (Roche®) was used to assess cell viability after conducting different treatment. TUNEL staining was co-stained with anti-α-actinin antibody so that cardiomyocyte-specific apoptosis could be assessed.

3.2 Methods to Assess Hepatocyte Maturation

The differentiation/maturation of hepatocytes was determined by RT-PCR to detect characteristic mRNAs that are expressed in hepatocytes including HNF4A, ALB, AFP, SLC10A1, APOB, APOH and ASGR.

3.3 Statistics

Data are expressed as mean±SEM. Differences were compared by ANOVA with Student-Newman-Keuls post hoc testing. P<0.05 was considered significantly different.

### 3. Results

3.1 AMPK Activation Enhances Fatty Acid Oxidation Capacity of hPSC-CMs

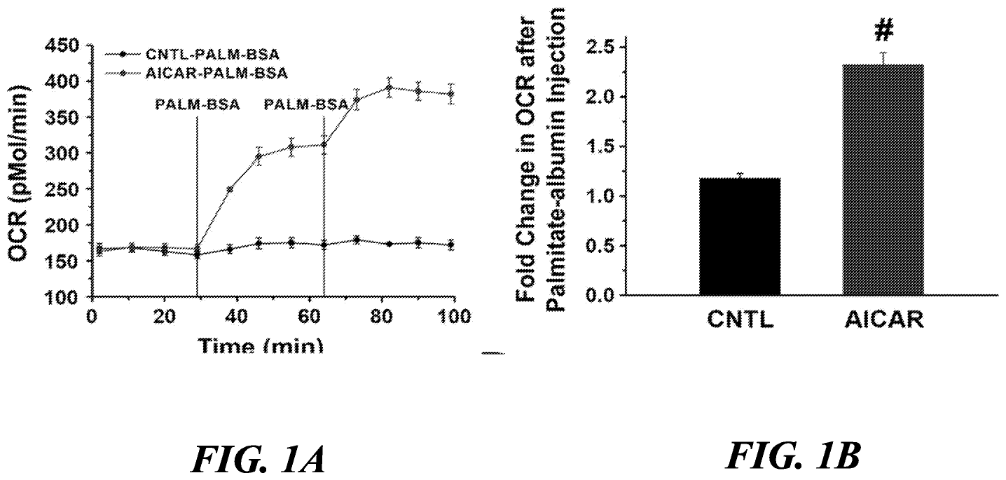

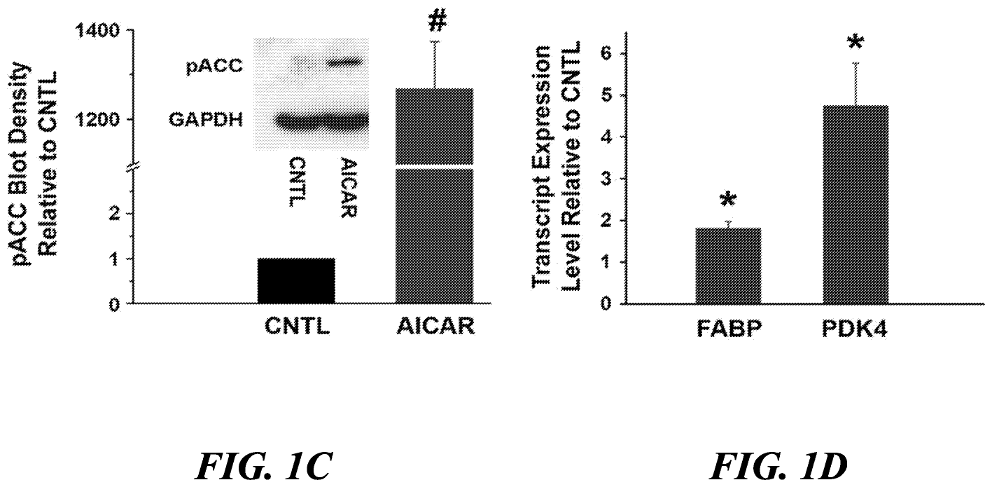

A hallmark during postnatal cardiomyocyte development is the metabolic switch from glucose to fatty acid oxidation for ATP production¹⁵. The immature hPSC-CMs rely heavily on glucose for their energy demands. First, it was investigated whether the untreated immature hPSC-CMs have the capacity to oxidize fatty acids and found that they, indeed, oxidize fatty acids at a small scale: an average 1.18±0.05-fold increase in oxygen consumption rate (OCR) upon palmitate-albumin introduction. After 2 weeks of AICAR-treatment, however, the OCR markedly increased to (2.32±0.12) fold (P<0.0001) (FIG. 1B). Representative traces are shown in FIG. 1A. In addition, the palmitate albumin XF96 assay was also performed in cardiomyocytes derived from the WTC hiPSC and RUES2 cell lines and similar results were observed.

During heart development, AMPK activation facilitates the metabolic transition from glucose to fatty acids by phosphorylating ACC, which then facilitates fatty acid transport into mitochondria and inhibits fatty acid biosynthesis. Thus, immunoblots were performed to detect levels of phospho-ACC (Ser 79). Under same exposure conditions, no detectable pACC expression was observed in the control cells but a more than one thousand-fold increase in pACC level after AICAR treatment (FIG. 1C), suggesting that, similar to what occurs during heart development, AMPK promotes fatty acids oxidation in hPSC-CMs at least partly by phosphorylating ACC.

In addition to phosphorylating ACC, AMPK activation also leads to a significant increase in the expression levels of some critical metabolic genes including fatty acid binding protein (FABP) and pyruvate dehydrogenase kinase-4 (PDK4) (FIG. 1D), in which the latter inhibits glucose and lactate metabolism. In addition, gene expression levels (FABP and PDK4) in cardiomyocytes-derived from RUES2 hESCs showed the same trend of changes.

3.2. Effect of AMPK Activation on Mitochondria Function and Biogenesis

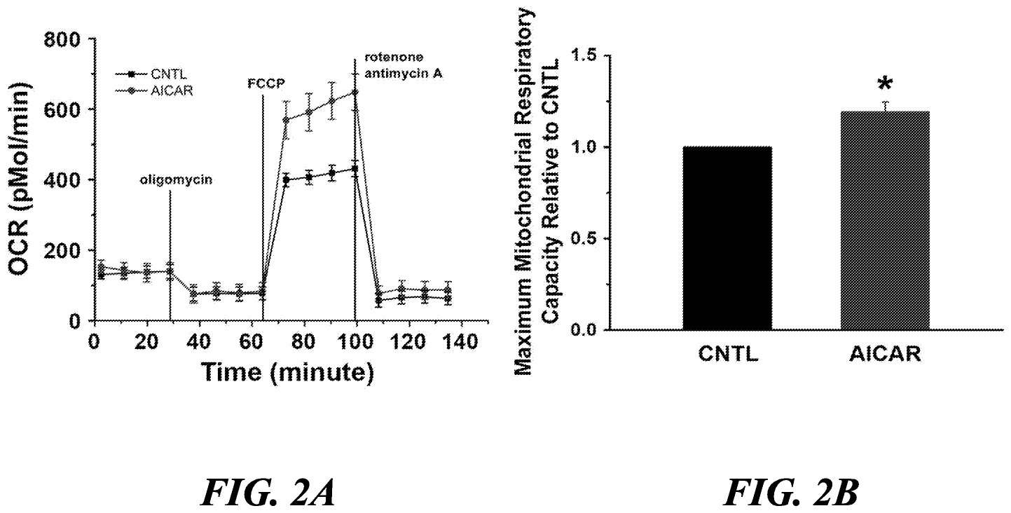

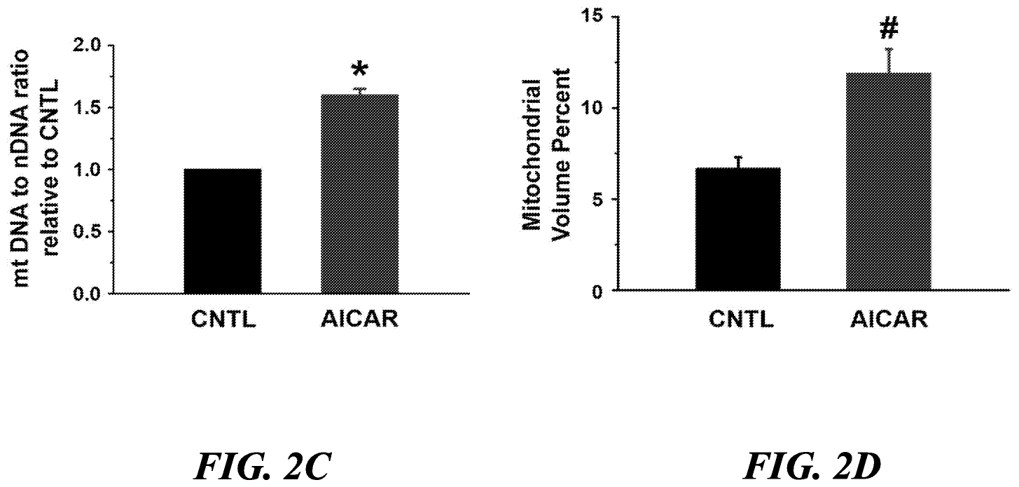

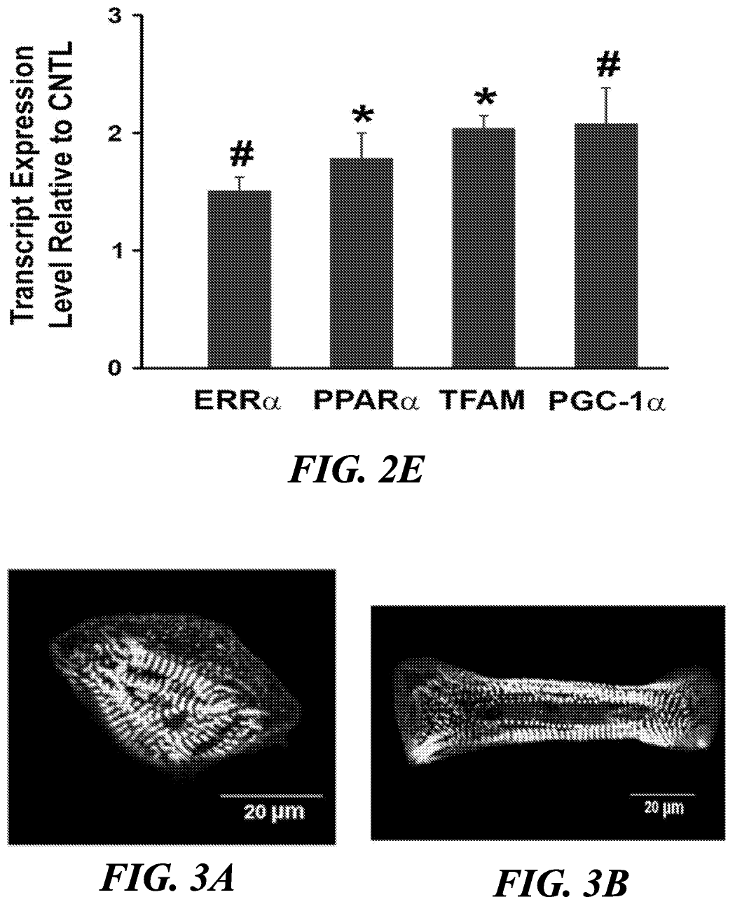

During development, mitochondria evolve both morphologically and functionally-, and it was contemplated that mitochondrial respiration and biogenesis would be stimulated by AMPK activation. Using the Seahorse XF96 extracellular flux analyzer, maximum respiration rate was measured by injecting the ATP synthase inhibitor, oligomycin, and a protonophoric uncoupler, FCCP. FIG. 2A shows representative traces of both control and AICAR-treated hPSC-CMs. AICAR treatment significantly increased maximum mitochondrial respiration capacity (FIG. 2B). AMPK activation also caused a 55% increase in the ratio of mtDNA to nDNA (FIG. 2C). The relative mitochondrial volume was calculated by point counting, in which 20 horizontal and 20 perpendicular lines were drawn in the images and the crosspoints that overlapped with mitochondria was counted and then divided by the total points. The analyses showed that mitochondrial volume fraction almost doubled after AICAR treatment (0.068±0.006 vs. 0.119±0.013, P<0.001)(FIG. 2D). Correspondingly, mRNA levels of transcriptional regulators of mitochondrial biogenesis such as estrogen-related receptor α (ERR α) (1.50±0.12, P<0.01), peroxisome proliferator activated receptor alpha (PPARα) (1.78±0.22, P=0.02), peroxisome proliferator-activated receptor gamma coactivator 1-alpha (PGC-1α) (2.07±0.31, P=0.02) and mitochondrial transcription factor A (TFAM) (2.03±0.11, P<0.0001) were increased by AMPK activation (FIG. 2E).

3.3 AMPK Activation Promotes Maturation of hPSC-CM Structure, Gene Expression and Myofibrillar Protein Isoform Switching

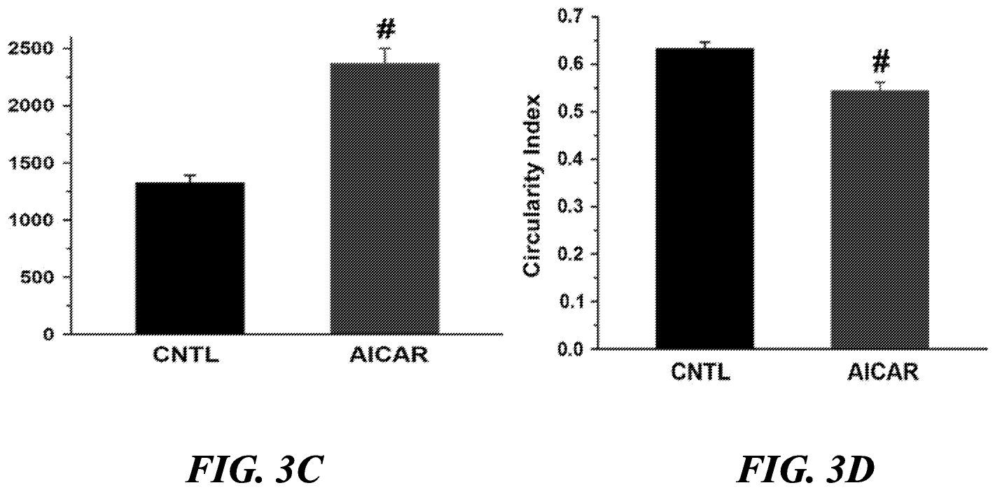

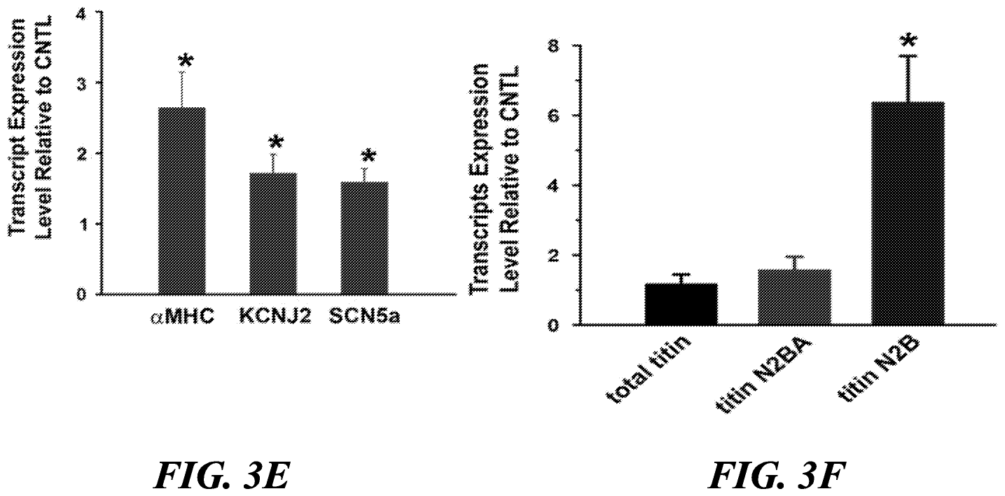

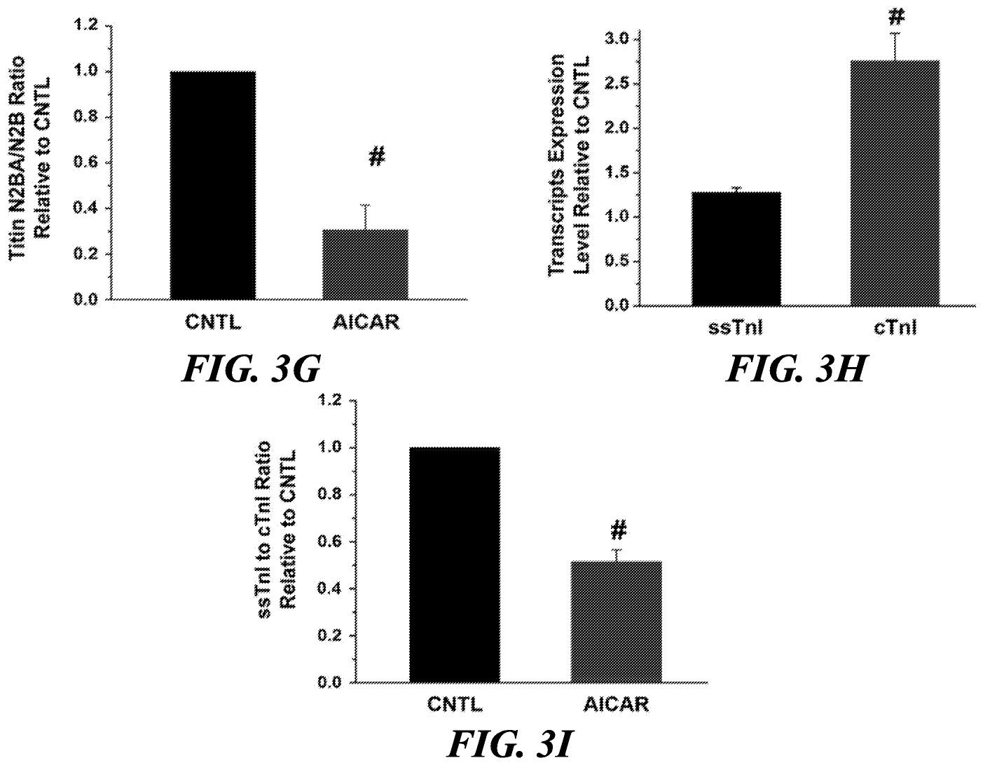

It was further investigated whether AMPK activation leads to changes in cellular morphology, since cardiac maturation leads to an increase in cell size and anisotropy. For these studies, the hPSC-CMs were immunocytochemically co-stained for α-actinin (green), F-actin (red), and DNA (Hoechst 33342). Untreated hiPSC-CMs were small and round to polygonal in shape (represented in FIG. 3A), consistent with previous reports³. A doubling in cell area or more was observed with AICAR treatment (FIG. 3B, C) (1141±40 μm² vs. 2321±120 μm², P<0.00001). To determine cardiomyocyte aspect ratio, the “circularity index” was assessed (Circularity=4π·Area/Perimeter²)⁴⁰. Under this assessment, “0” represents a theoretical minimum for perfect rod-shaped cells (actually a line with no area), with “1” for cells that are perfectly circular. AICAR-treatment resulted in a decreased circularity index (0.51±0.01 vs 0.40±0.02, P<0.00001) (FIG. 3D), indicating that the hPSC-CMs exhibited a more mature morphology.

Quantitative RT-PCR analysis of the expression level of various cardiac genes were also performed on control and AICAR-treated cells. It was observed that AICAR resulted in the up-regulation of α-myosin heavy chain (α-MHC) (2.64±0.51-fold vs. control, P=0.03), KCNJ2 expression (1.71±0.17-fold vs. control, P=0.04), and SCN5a (1.58±0.2-fold vs. control, P=0.02) (FIG. 3F).

During development, several myofibrillar proteins exhibit a functionally-relevant isoform-switch which modulates the contractile function of cardiomyocytes. For instance, Titin is involved in the maintenance of sarcomere integrity and elasticity. Titin shifts from a relatively compliant isoform with a size range˜3200-3700 kDa (designated N2BA) to a stiffer isoform (3000 kDa, designated N2B), which results in an increased passive tension of maturing cardiomyocytes²⁹. In human postnatal left ventricles, N2B is the dominant form. As shown in FIG. 3F, total cardiac titin mRNA levels were very similar to the levels of N2BA mRNA. Much less N2B than N2BA mRNA was detected in both groups. While AICAR treatment did not change the expression level of total titin and titin N2BA, titin N2B expression level was significantly upregulated. As a result, titin N2BA to N2B ratio was significantly down-regulated after AICAR treatment (FIG. 3G), suggesting the AICAR-treated hPSC-CMs may generate more passive tension than the control hPSC-CMs.

Troponin I also undergoes a developmentally regulated isoform switch. Human fetal hearts contain both isoforms: slow skeletal troponin I (ssTnI) and cardiac troponin I (cTnI), whereas adult hearts exclusively express cTnI⁴¹. The troponin complex containing cTnI has decreased Ca²⁺ sensitivity for tension production, compared with complexes containing ssTnI⁴². This ssTnI to cTnI switch has been proposed as a quantitative ratiometric marker for cardiac maturation¹². Q-RT-PCR assay revealed that cTnI expression levels were upregulated after AICAR treatment (FIG. 3H). And the ssTnI to cTnI ratio was significantly downregulated (FIG. 3I).

3.4 AMPK Activation Enhances Contractile Force and Reduces Automaticity

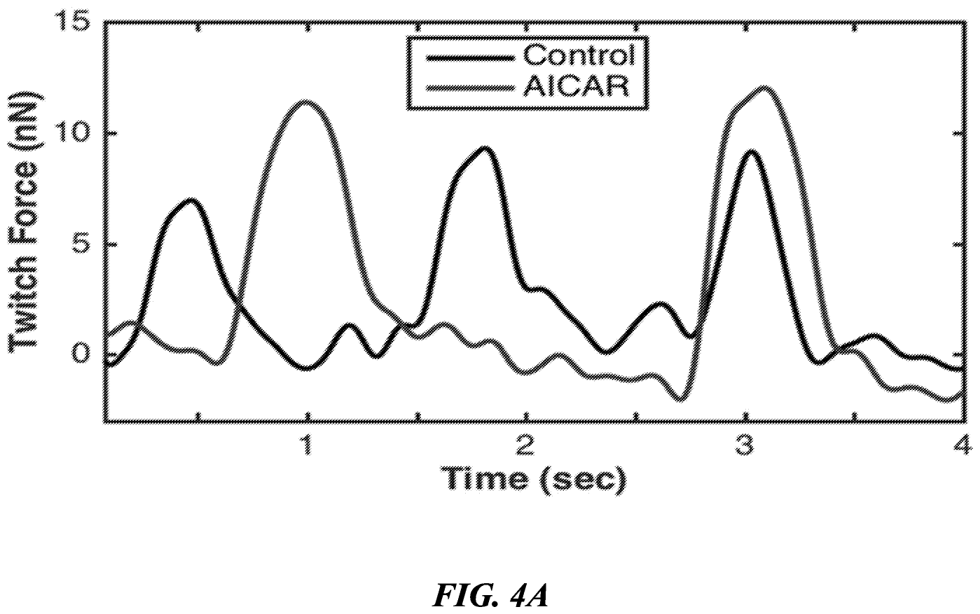

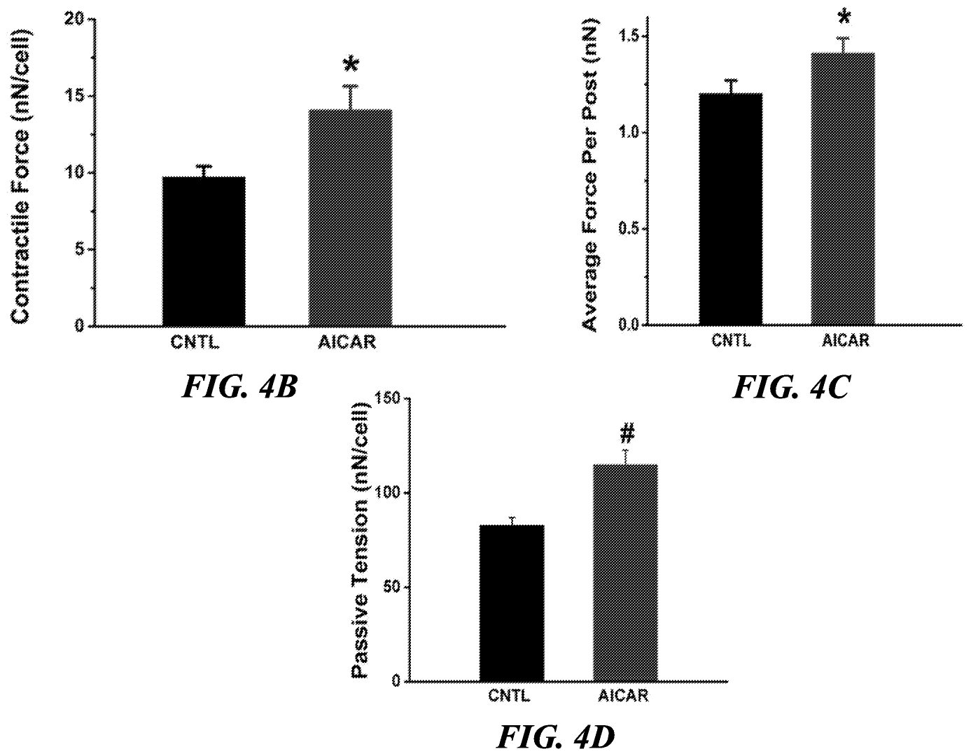

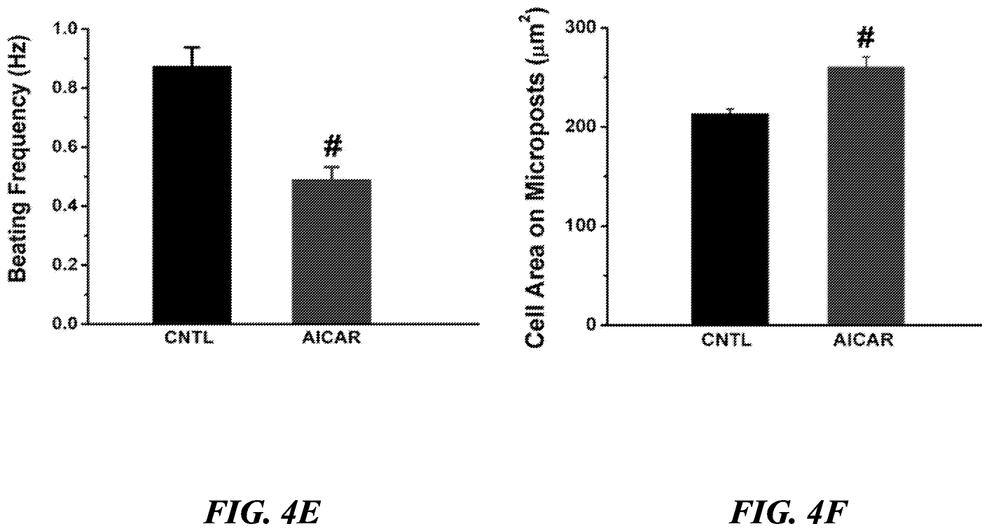

Based on the morphological observations described above, it was contemplated that that AICAR treatment would increase contractile force. To characterize force production on a per-cell basis a micropost array system was used^{2, 31}. For this approach, individual cardiomyocytes were allowed to adhere to elastomeric microposts. As the cardiomyocytes contract, the deflections of the posts underneath a cell were recorded. By modeling each post as a cantilever beam, the forces produced at each adhesion can be calculated from Hooke's law (eq. 1 as shown in session 2.8). The magnitude of the force vectors can be summed to obtain the total contractile force produced by a cell at each time point. FIG. 4A shows representative traces of the total twitch force generated by individual cardiomyocytes from the control and AICAR-treated groups. Control hiPSC-CMs exhibited a twitch force of 9.7±0.7 nN/cell (FIG. 4B). AICAR-treated hiPSC-CMs exhibited a significantly higher twitch force of 14.1±1.6 nN/cell (P=0.014). Similarly, AMPK activation significantly increased the local force generated at each post (FIG. 4C). In addition to the active twitch force, the passive tension generated by the AICAR-treated hPSC-CMs was significantly higher than the controls (82.68±4.35 nN/cell for control cells vs 114.77±7.93 nN/cell after AICAR treatment) (FIG. 4D). The observed increase in passive tension is likely attributed to a significantly lower N2BA to N2B ratio in AICAR-treated hPSC-CMs (FIG. 3G). The beating frequency analysis also showed that AICAR-treated cells beat significantly slower (0.87±0.07 Hz for control vs 0.49±0.04 Hz after AICAR treatment) (FIG. 4E). Lastly, in agreement with the morphological changes after AICAR treatment shown in FIG. 3A, the hPSC-CMs on microposts also displayed a larger cellular size (FIG. 4F) (213±5 μm² vs 260±11 μm², P<0.001). These data not only show that AICAR-treatment results in morphological and molecular changes indicative of maturation, but that functionally relevant parameters, such as those associated with contraction, are also positively regulated.

3.5 AICAR Treatment Activates Multiple Intracellular Signal Pathways

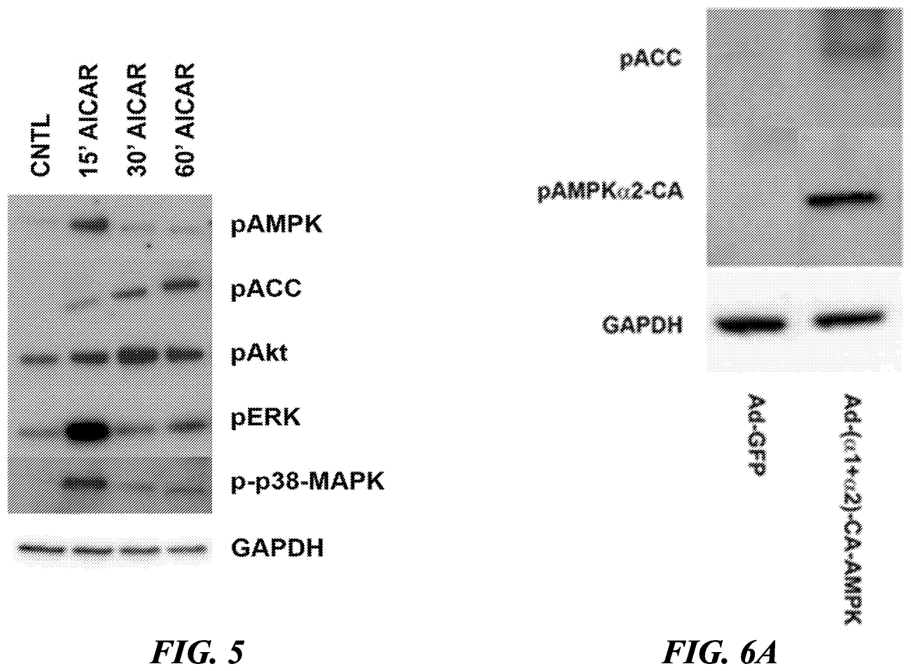

To investigate possible intracellular signal pathways activated by AICAR treatment, immunoblotting analysis with phospho-specific antibodies to multiple kinases were performed. Firstly, it was confirmed that 1 mM AICAR treatment leads to a rapid robust transient phospho-Thr172-AMPK phosphorylation⁴³. Also, treatment with 1 mM AICAR caused a rapid, time-dependent increase in the phosphorylation of Ser79-ACC, a well-characterized substrate of AMPK. These results verified that AICAR treatment activated AMPK in hPSC-CMs. AICAR treatment also increased the phosphorylation of Akt at Ser473. Mitogen-activated protein kinase (MAPK) pathways including c-Jun N-terminal kinase (JNK), extracellular signal-regulated kinase (ERK), and p38 are involved in cellular proliferation and growth. One study⁴⁴ showed that AMPK activation by AICAR in L6 myotube leads to ERK phosphorylation. Transient increases were also observed for phospho-ERK and phopho-p38-MAPK following AICAR treatment (FIG. 5).

3.6 Viral Transfection Confirms Major AICAR Findings in hPSC-CMs

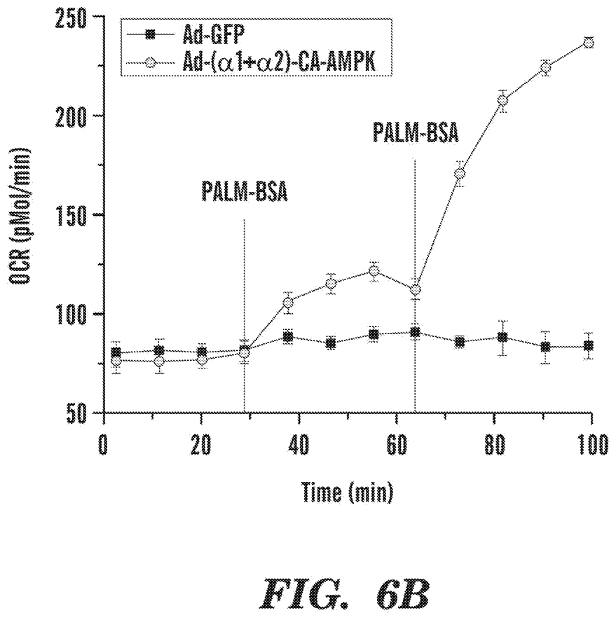

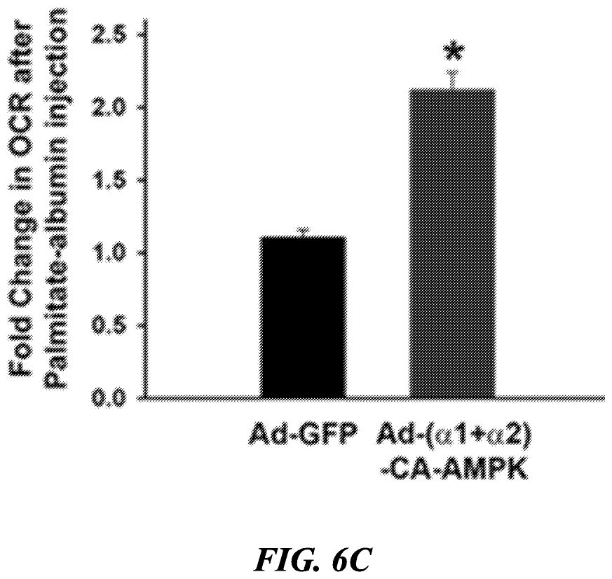

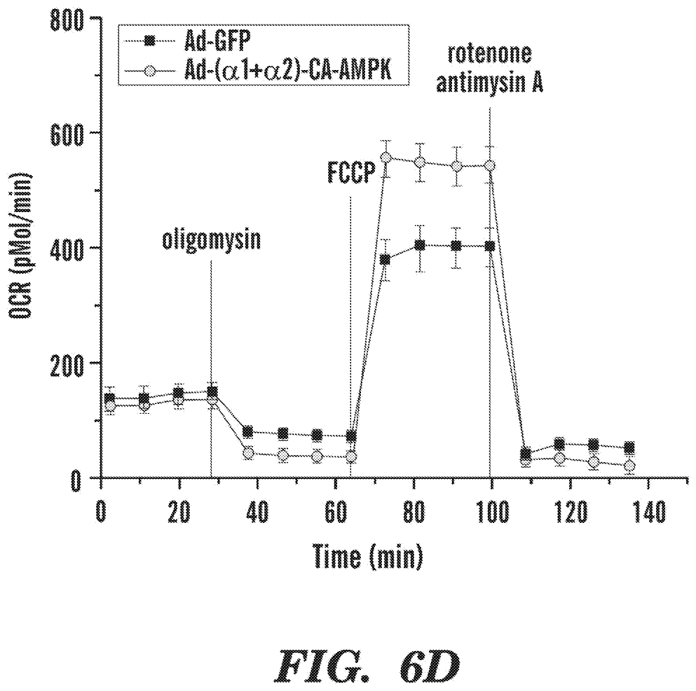

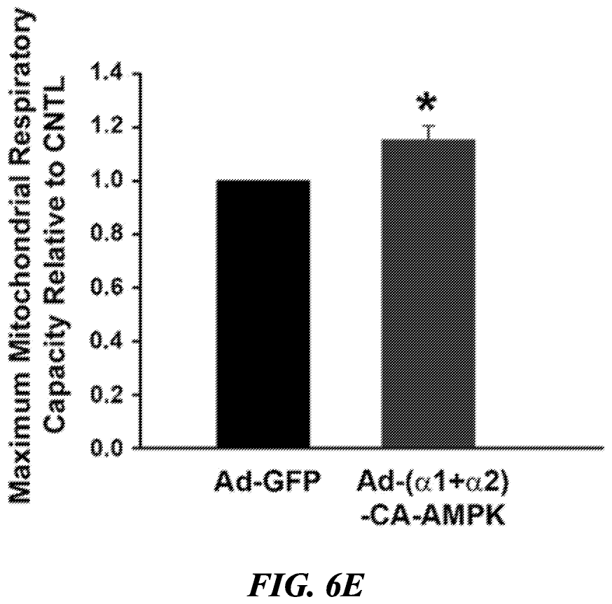

AMPK is a heterotrimeric complex consisting of a catalytic α subunit and regulatory β and γ subunits. The α-subunit exists in two isoforms, α1 and α2, encoded by separate genes, and the α-subunit contains the AMPK serine-threonine kinase domain, which has a critical activating residue within the catalytic cleft (Thr¹⁷²). The phosphorylation status of this amino acid by upstream kinases is essential for AMPK activity, and its phosphorylation status often is used as an indicator of the activation state of the kinase. In cardiac muscle⁴⁵, α2 AMPK complexes counted for 70-80% of total AMPK activity while α1 complexes accounted for the remaining 20-30%. In this study, hPSC-CMs were transduced simultaneously with adenoviruses encoding both constitutive-active AMPKα1 and α2 to verify the major metabolic findings after AICAR treatment. Viral transfection led to an increase in Ser79 ACC phosphorylation and phosphorylation of truncated AMPK-α2 (˜35 kDa), confirming that viruses activated AMPK (FIG. 6A). In the palmitate albumin extracellular 96 flux assay, a significant increase in palmitate oxidation was observed (2.32±0.12 vs. 1.18±0.05) (FIGS. 6B and C) after viral transduction. Lastly, viral transduction also significantly increased maximum mitochondrial respiration capacity (1.16±0.05 times of control) (FIGS. 6D and E). These findings with constitutively active AMPKα1 and α2 subunits confirmed the major metabolic findings with AICAR treatment, indicating that the observed AICAR effects result from AMPK activation rather than from other unspecific pharmacological effects.

3.7 AMPK Activation Induces ARVD Pathologies

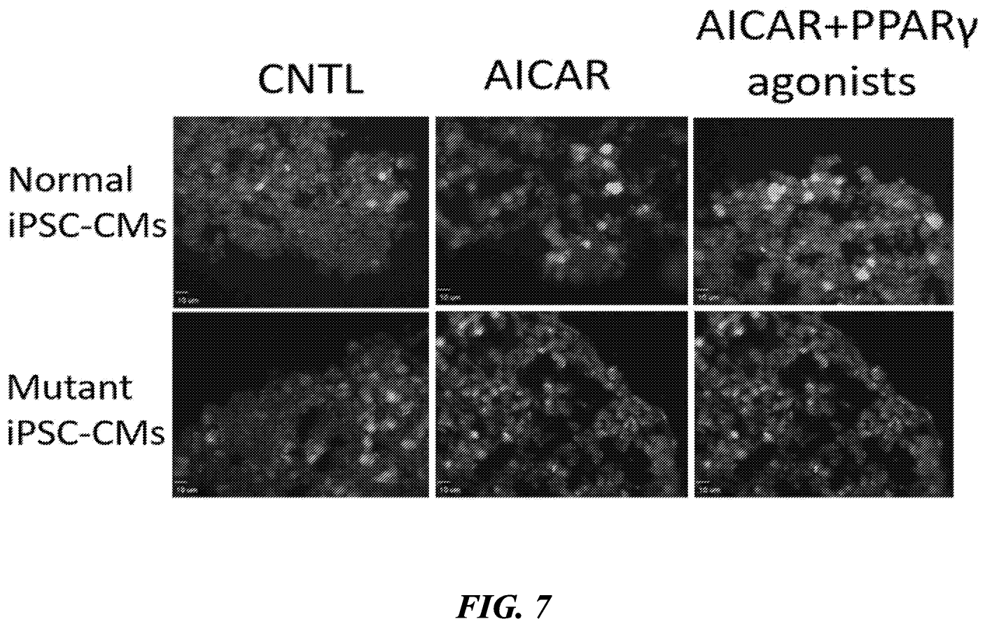

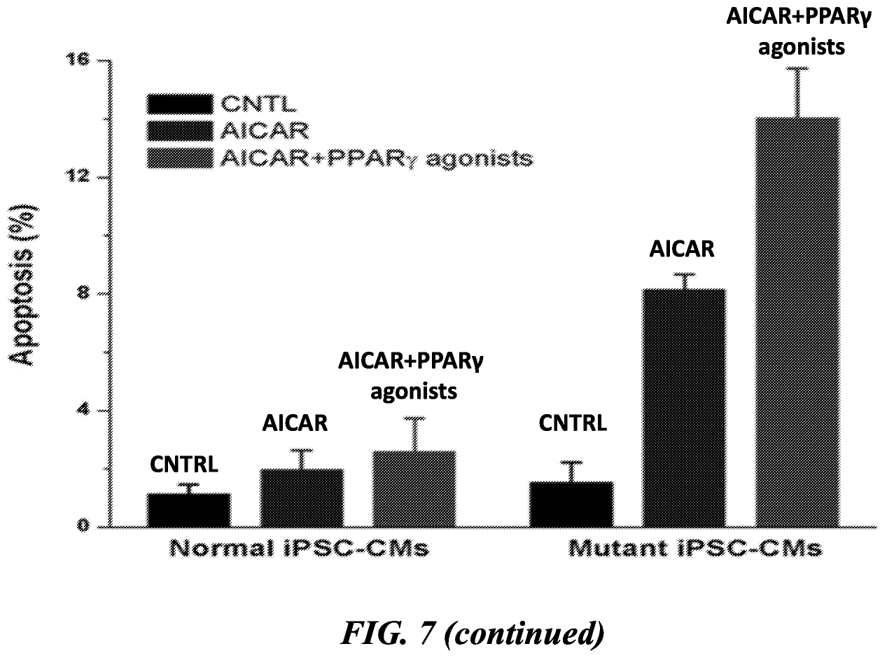

Arrhythmogenic right ventricle dysplasia/cardiomyopathy (ARVD/C) is an inherited heart disease characterized by pathological fatty infiltration and cardiomyocyte loss predominantly in the right ventricle³⁷. It is an adult-onset disease. The cardiomyocytes derived from ARVD/C hiPSCs did not reproduce the pathological phenotypes of ARVD/C unless the cardiomyocytes were induced to switch from an embryonic/glycolytic state to fatty acid oxidation³⁷. Considering the major effects of AMPK activation on fatty acid oxidation, the effect of AICAR on ARVD/C hiPSC-derived cardiomyocytes was explored. Treatments groups included AICAR, and AICAR plus PPAR-gamma agonists (5 μM rosiglitazone and 200 μM indomethacin). None of the treatment groups significantly leads to apoptosis in the control cardiomyocytes. In the cardiomyocytes from ARVD/C hiPSCs, AICAR alone induced significant apoptosis (FIG. 7). The addition of PPAR-gamma agonists leads to further cell death, indicating that there is an additive effect on cell apoptosis.

3.8 AMPK Activation Increases Neuronal Mitochondrial Biogenesis, Promotes Neuronal Maturation, and Reduces Secreted Amyloid Beta Peptides in hiPSC-Derived Neurons.

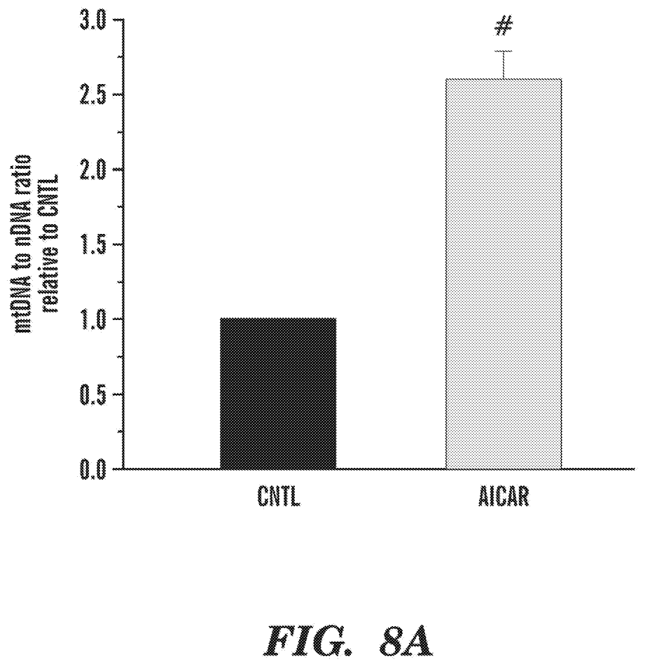

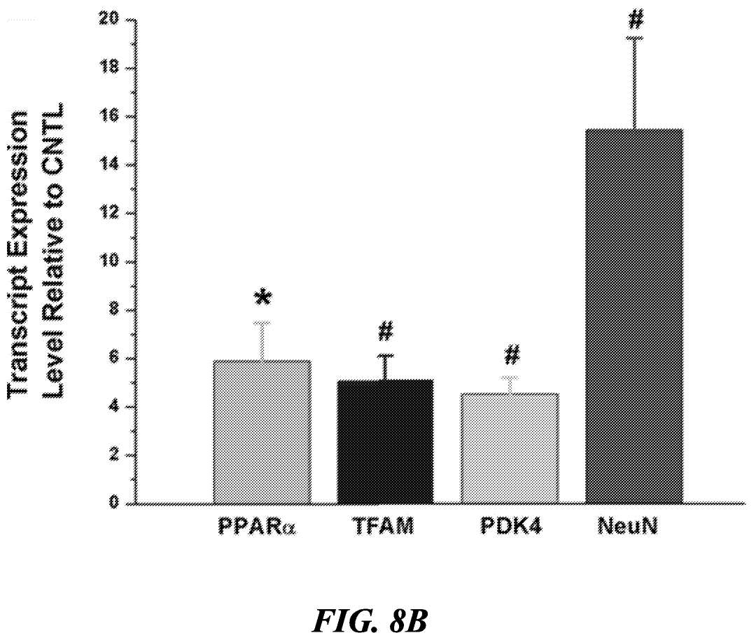

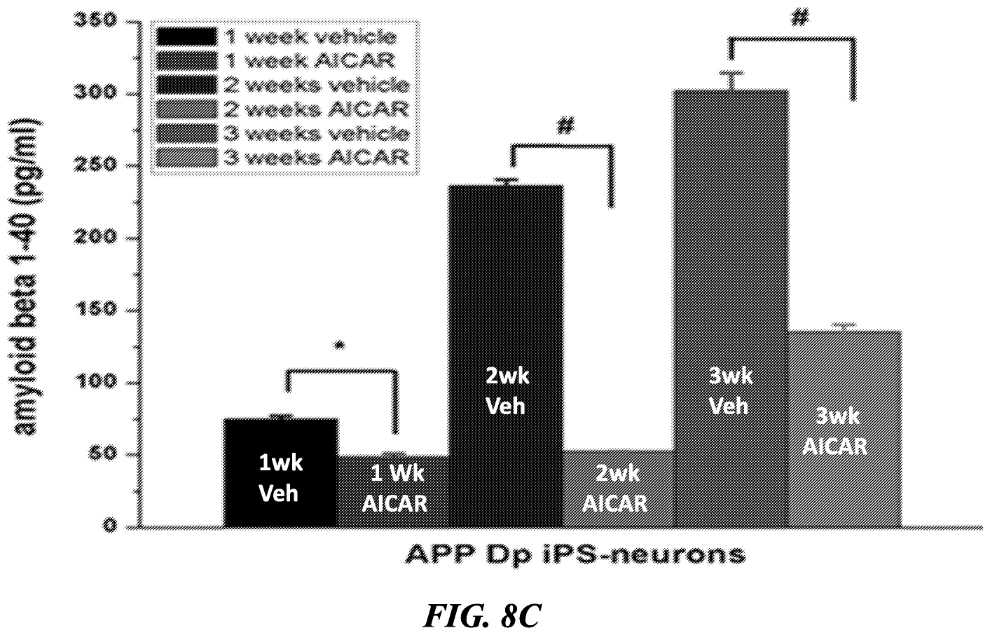

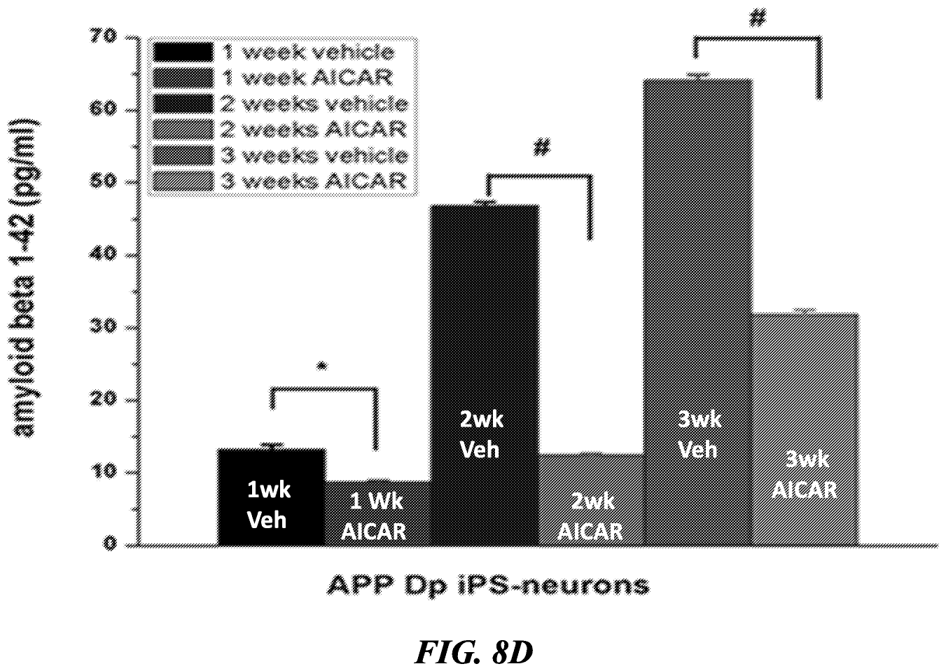

Neurons are highly metabolic cells that rely on oxidative phosphorylation for energy production. However, little is known how this process develops during neuronal maturation. Recent work has demonstrated that hiPSC-derived neurons increase mtDNA and up-regulate metabolic and mitochondrial gene transcription during differentiation process¹⁶. To test whether AMPK activation enhances mitochondrial biogenesis in hiPSC-derived neurons in a similar manner to hiPSC-derived cardiomyocytes, the effect of two weeks of treatment with AICAR every third day was assessed. Consistent with what was observed in hiPSC-derived cardiomyocytes, an increase in mtDNA/nDNA ratio was discovered (FIG. 8A) and in genes regulating mitochondrial biogenesis and metabolism (FIG. 8B). Interestingly, an increase in PDK4 was also observed, which is involved in regulation of glucose and fatty acid metabolism, although previous work reported a decrease in PDK4 during normal neuronal differentiation⁴⁶. This suggests that activating AMPK by AICAR during neuronal differentiation may push cells away from a glycolytic metabolic state. Finally, the levels of NeuN (Neuronal nuclei protein, FOX3) were analyzed which is a marker present in nearly all neuronal types and an indicator of neuronal maturation⁴⁷. A significant increase in NeuN mRNA in neurons treated with AICAR as compared to control neurons was observed, suggesting that activating AMPK may promote maturation in hiPSC-derived neurons.

Human iPSCs-derived neurons are a powerful model for many neurologic diseases including Alzheimer's disease (AD)⁴⁸. The hallmark pathological molecules, Amyloid beta (Aβ) and phosphorylated Tau protein can be detected from hiPSC-derived neurons. Cell lines generated from patients with early-onset, familial AD (FAD) show an increases in these toxic proteins^{27, 49, 50}. Recent work has implicated neuronal metabolic dysfunction with AD pathophysiology^{51, 52}. Therefore, it was tested whether activation of AMPK by AICAR affects cellular phenotypes relevant to AD pathology from a FAD patient cell line harboring a duplication of the amyloid precursor protein (APP gene). This mutation leads to early-onset AD in patients and hiPSC-derived neurons generated from these patients have been previously shown to have elevated levels of secreted Aβ peptides^{27, 53}. These results show that treatment with AICAR during a three-week neuronal differentiation from neural stem cells significantly reduced the levels of Aβ peptides secreted (both Ab₁₋₄₀ and the more pathogenic Ab₁₋₄₂) from FAD neurons at each week of the differentiation. (FIGS. 8C and 8D). Taken together, these data suggest that enhancement of neuronal mitochondrial metabolism and maturation by AMPK activation may be a pathway of interest for AD treatment.

3.9 AMPK Activation on Hepatocyte Maturation

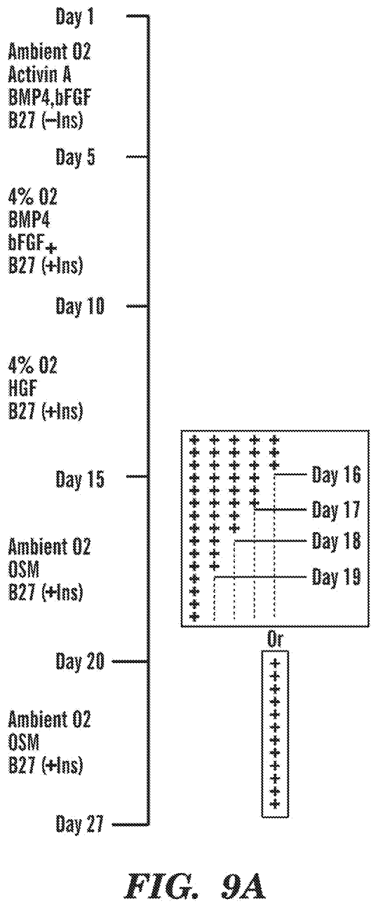

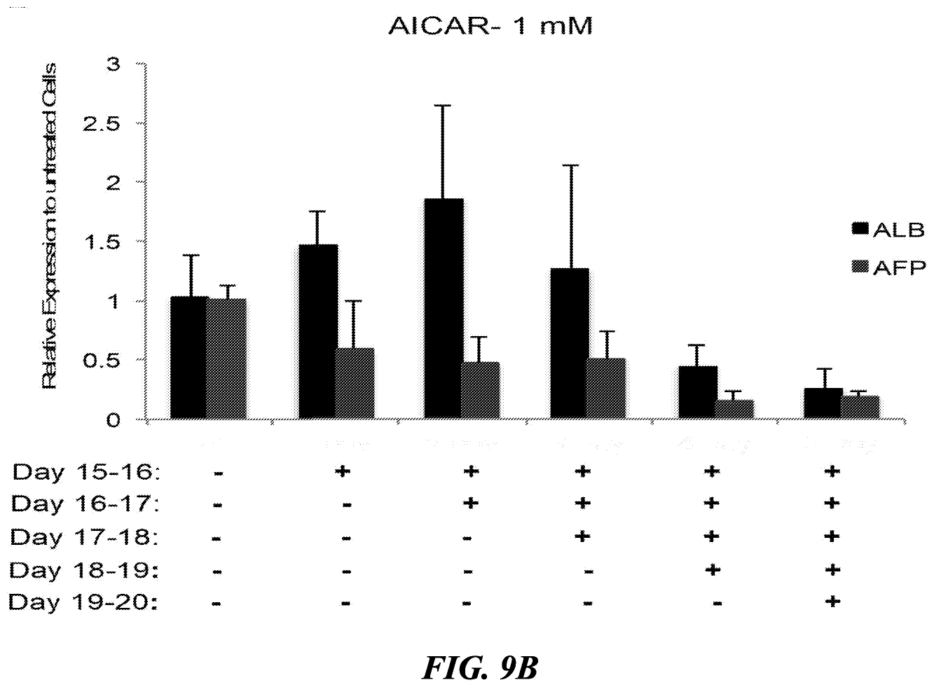

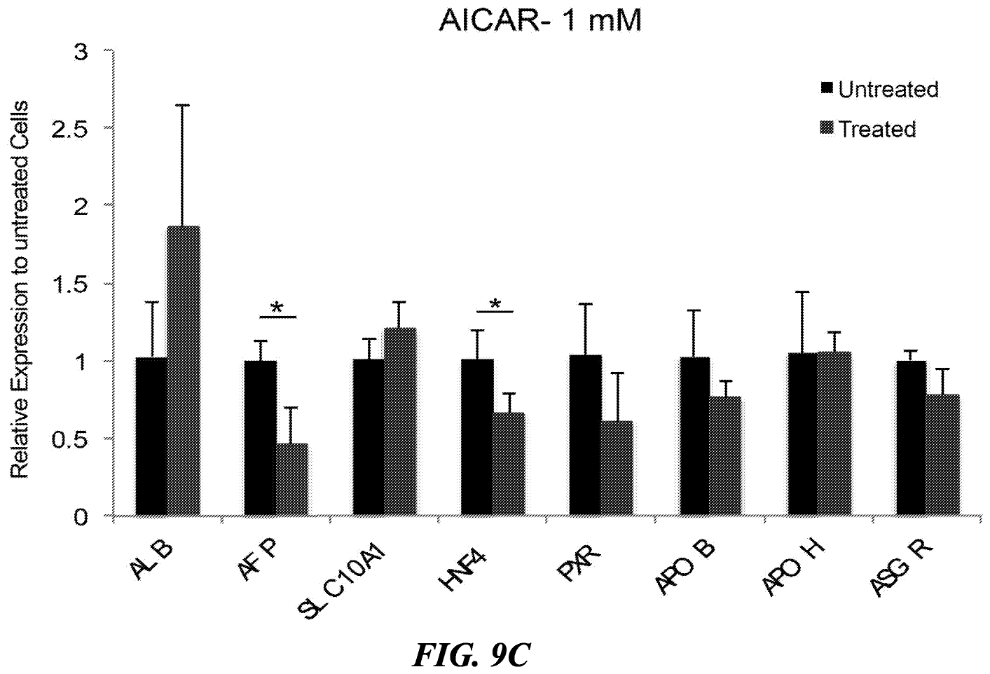

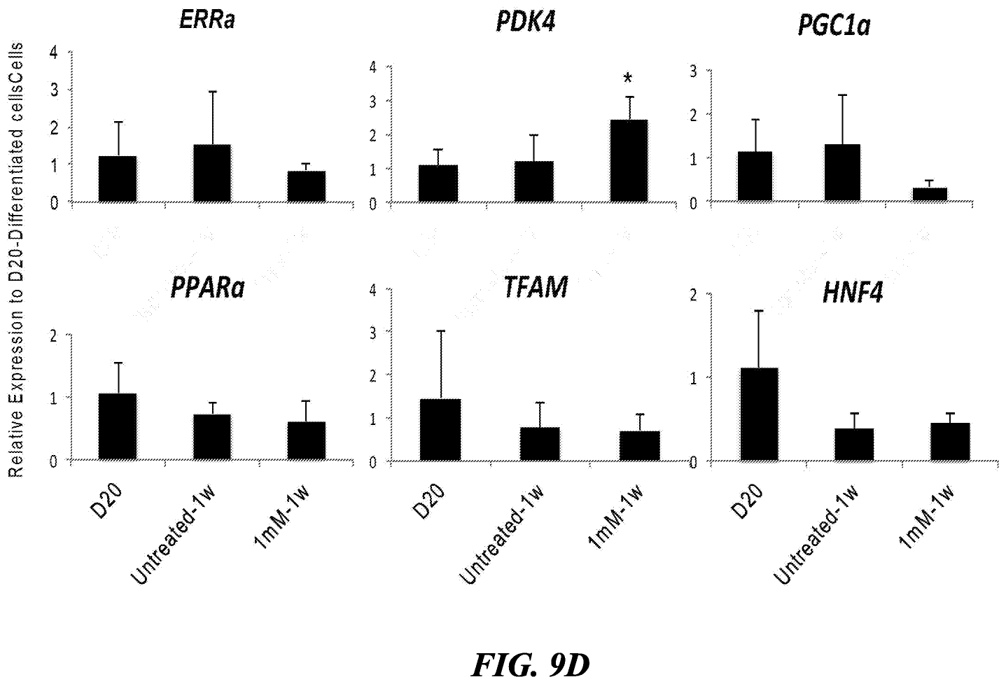

To investigate whether the improved phenotype observed in cardiomyocytes and neurons is applicable to other cell types, the impact of AICAR on the production of hepatocyte-like cells (HCLs) from hiPSCs was examined (FIG. 9A). HLCs are an attractive candidate for AICAR treatment as they do not reach full maturity in vitro, require high levels of ATP production from the mitochondria⁵⁴, and PGC1α, a downstream effector of the AMPK signaling pathway, has previously been shown to regulate the expression of HNF4α, a key hepatic transcription factor during development⁵⁵.

AICAR was added to the medium at a concentration of 1 mM for 1 to 5 days between days 15 and 20 of differentiation and quantitative PCR evaluation of ALB and AFP on day 20 differentiated cells was used to assess hepatic maturation. The relative ratio of Albumin to AFP is a useful indicator of the maturation of hepatocytes because AFP and Albumin expression are generally restricted to fetal and adult hepatocytes, respectively. Endpoint analysis at day 20 showed that longer treatment with AICAR adversely affected ALB expression. Conversely, treatment with AICAR from day 15 to 17 resulted in enhanced ALB and diminished AFP expression; however, this was found to be non-significant (FIG. 9B). Further analysis on this group showed no significant difference in other mature hepatic-related genes (SLC10A1, APOH, ASGR, APOB, and PXR). Moreover, the important hepatic-related transcription factor HNF4α, was significantly down-regulated by AICAR treatment (FIG. 9C). These results suggest that treatment with AICAR during the maturation step of HLCs does not significantly improve hepatic gene expression.

Following differentiation, HLCs, like cardiomyocytes, remain immature, limiting their usage. As AICAR supplementation promotes the mature phenotype of cardiomyocytes, it was likely that HLCs can be impacted through similar mechanisms. Therefore, HLCs were treated at day 20 with 1 mM AICAR for an additional seven days.

As the effect of AICAR in cardiomyocytes is dependent on the up-regulation/maintenance of mitochondrial biogenesis regulators, these same factors were evaluated (PGC1α, ERRa, PDK4, PPARα, and TFAM) in HLCs, alongside HNF4α as a non-mitochondrial indicator of hepatic maturity. These results showed that most of the selected genes, including HNF4α, reduced non-significantly following an additional 7 days of culture and that AICAR treatment had no significant effect. The only gene that significantly increased with AICAR treatment was PDK4, which is a regulator of glucose metabolism. These results indicate that AICAR treatment does not improve the maintenance of the mature HLC phenotype and does not induce the same expression changes in mitochondrial biogenesis genes as observed in cardiomyocytes and neurons (FIG. 2 and FIG. 8).

### Discussion

In the last few years, the development of efficient directed differentiation protocols has enabled the generation of diverse cell types derived from hPSCs. The next-level challenge is to promote the maturation of these hPSC-derived, so as to maximize their impact for regenerative therapies, disease modeling, and drug/toxicity screening. Maturation is a complex trait. For instance, heart development involves structural, biochemical, electrical, and mechanical signals that undergo dynamic changes. Recently, progress has been made in obtaining hPSC-CMs with more adult-like morphology that display better contractile force and more mature calcium handling properties using diverse approaches such as prolonged culture periods^{3,12}, mechanical conditioning¹⁴, thyroid hormone treatment², or microRNA^{2,3, 10-12}. Metabolically, however, hPSC-CMs derived using current methods have only modest mitochondrial capacity and heavily rely on glucose, adult cardiomyocytes, however, obtain the majority of the energy from fatty acid oxidation. In this investigation, promoting hPSC-CMs metabolic maturation by activating what has been considered to be a “super metabolic regulator” AMPK¹⁶ was the focus.

At baseline, control hPSC-CMs showed only a modest increase (˜1.2-fold increase) in OCR upon palmitate-albumin injection. It is likely that this low OCR increase results from hPSC-CMs containing immature mitochondria that are not fully functional and do not possess sufficient levels of enzymes involved in fatty acid oxidation. After AICAR treatment, however, the OCR-induced by palmitate-albumin went up by ˜2.3-fold. The rate-limiting step of fatty acid oxidation (FAO) is the import of long chain fatty acids across the mitochondrial membrane through CPT1. CPT1 activity is strongly inhibited by malonyl CoA, which is formed by the carboxylation of acetyl CoA via ACC. AMPK activity increases during postnatal heart development¹⁵ and its activity augments the phosphorylation of ACC, which subsequently inhibits malonyl CoA production, facilitating the energy substrate transition from carbohydrate to fatty acids. It was shown here that in hPSC-CMs AMPK activation results in robust phosphorylation of ACC at Ser79. This suggests that AMPK increases FAO partly by facilitating fatty acids import through a mechanism involving decreased malonyl CoA production via ACC phosphorylation. It was also found that AMPK activation leads to increased mRNA level of genes involved in FAO, and increased mitochondrial biogenesis. The net results of these changes are enhanced FAO and mitochondrial maximal respiratory capacity.

AICAR treatment increases the twitch force generation from ˜9.7 nN/cell to ˜14.1 nN/cell, as assessed by the established micropost assay^{2, 32}. Since the AICAR-treated cardiomyocytes were significantly larger than the controls, the average twitch force per post to account for the role of hypertrophy in the total increased contractile force were compared. The average twitch force per post was also significantly higher in the AICAR-treated cardiomyocytes (˜1.4 nN/post) compared to the controls (˜1.2 nN/post). Therefore, other factors besides increased cell size must also contribute to the increased force. The decreased beating frequency of the AICAR-treated hPSC-CMs on microposts also suggests a positive role of AMPK activation in maturation. One characteristic property of an immature cardiomyocyte is automaticity, which diminishes during cardiac development. The decreased beating rate may result from the higher expression level of the inwardly rectifying potassium channel (Kir) subunit KCNJ2, which is important in setting resting membrane potential.

Another interesting finding is that AMPK activation facilitates titin and troponin I isoform switching toward an adult phenotype. A molecular spring, titin is a main player in determining passive muscle mechanics. Before birth, when the compliance of the heart is restricted by extracardiac constraint⁵⁶, a N2BA isoform is expressed to keep titin-based passive tension low. At birth, the heart suddenly experiences much-increased filling pressures and significantly reduced extra-cardiac constraint. Therefore, to limit the extensibility and increase titin-based passive tension, N2B isoform expression is quickly upregulated in the newborn heart. Currently, it is not well known how the titin isoform shift is regulated. RNA binding motif 20⁵⁷, a gene for hereditary cardiomyopathy, and thyroid hormone⁵⁸ have been shown to regulate titin splicing. However, no previous work has reported the interaction between AMPK and titin isoform expression levels. In agreement with increased titin N2B expression level, an increased passive tension was generated by AICAR-treated hPSC-CMs.

To assess whether AICAR actually activates AMPK in this study and to explore the possible intracellular signal pathways activated by AICAR treatment, immunoblots against several key phospho-kinases were performed. It was found that AICAR leads to transient AMPK (Thr172) and consistent ACC (Ser79) phosphorylation, demonstrating that AICAR activates AMPK. Immunoblots also showed that AMPK activation leads to Akt phosphorylation and transient ERK and p38-MAPK phosphorylation. A previous study⁵⁹ suggests that Akt phosphorylation leads to AMPK inhibition by modulating cellular energy homeostasis through maintaining cellular ATP levels, which may provide explanation of the transient AMPK phosphorylation after AICAR treatment. Phospho-p38-MAPK may serve as an intermediate kinase between AMPK and PPARα transcriptional activity⁶⁰. The increased p38-MAPK phosphorylation after AICAR treatment may be directly involved in regulating the transcriptional activity of PPARα, which subsequently leads to upregulation in the expression level of genes involved in fatty acid oxidation (such as FABP as shown in FIG. 1D). Also, p38 MAPK is known to phosphorylate PPARα and increase its association with a coactivator PGC-1α⁶¹. These results suggest the presence of multiple pathways that could mediate the effect of AICAR (AMPK activation) on fatty acid metabolism in hPSC-CMs. To further test the specificity of the role of AMPK, key metabolic findings were confirmed using adenoviruses encoding constitutively active forms of AMPKα1 and AMPKα2.

Modeling an adult-onset heart disease with hPSC-CMs remains a significant challenge due to the immature properties of these cells. A recent study by Kim et al highlights the need for more mature hPSC-CMs for disease modeling³⁷. This group derived hPSC-CMs from patients with arrhythmogenic right ventricular dysplasia/cardiomyopathy (ARVD/C), containing mutations in the plakophilin-2 (PKP2) gene. Pathological phenotypes such as exaggerated lipogenesis and apoptosis were observed only after the cells were induced to switch from an embryonic/glycolytic state to fatty acid oxidation. In this study, treatment with over a 4 to 5 weeks period was necessary to achieve the desired phenotype. Considering the profound effect of AMPK activation on cardiomyocyte metabolic maturation, it is possible that activating AMPK in the PKP2 hiPSC-CMs for a shorter amount of time would lead to the observed phenotype. Not surprisingly, two weeks of AMPK activation should also help to improve the modeling of other metabolic-related cardiovascular diseases.

hiPSC-derived neurons have great potential to contribute to disease modeling, therapeutic development, or cell replacement for neurologic disease. However, the generation of mature neurons from hiPSCs is still challenging and variable among different protocols. Long-term culture (up to over one year) or co-culture with astroglial cells are methods that have been successful in obtaining neurons with mature ion current and electrophysiological properties^{72, 73}. For hiPSC-derived neurons, AMPK activation increased mitochondrial biogenesis and metabolism markers as well as a canonical marker of neuronal maturation, NeuN. This data supports previous work describing metabolic reprogramming during neuronal differentiation⁴⁶ and suggests that activation of AMPK during this time may enhance this process. As major producers of cellular energy, mitochondria are essential for neurogenesis, neurite outgrowth, and synaptic plasticity in neurons and multiple signaling molecules regulate both mitochondrial biogenesis and neuroplasticity⁷⁴. Therefore. it is likely that molecules promoting cellular metabolism, such as AICAR, may promote functional neuronal maturation from hiPSCs. This is promising not only in terms of understanding neuronal metabolism during differentiation, but for modeling of age-related neurodegenerative disease where mature neurons may more accurate represent cellular phenotypes present in an adult brain.

In terms of AD, the current failure rate of clinical trials is very high⁷⁵, therefore identification of pathways, such as enhancers of cellular metabolism, using human-specific neurons may provide new avenues for therapeutic development. While specifically targeting Ab has not resulted in an effective therapy for AD, this molecule is central to AD pathology and an established biomarker of the disease. Treatment of stem cell-derived neurons with inhibitors of enzymes involved in APP processing demonstrated markedly reduced levels of Ab²⁷ and neurotrophic molecules also reduce secreted Ab peptides in neurons from AD patients^{27,28}. Recently, the potential of hiPSC-derived neurons as a preclinical model was demonstrated by phenotypic screening that identified modulators of APP processing by the analysis of differing lengths of Ab peptides⁷⁶. Here it was demonstrated that treatment of AD patient derived neurons with AICAR reduces Ab peptides, including the highly pathogenic Ab₁₋₄₂, secreted from hiPSC-derived neurons over the course of a three-week neuronal differentiation. Taken together, this suggests that hiPSC-derived neurons are highly amenable to high-throughput applications to identify small molecules that have significant effects on established disease biomarker.

Taken together, the robust metabolic effect of AMPK as shown herein allows for activators of AMPK to control hPSC-CM and neuronal maturation, and also enhance disease modeling in cardiac and neuronal systems.

### REFERENCES

1. Yang X, Pabon L and Murry C E. Engineering adolescence: maturation of human pluripotent stem cell-derived cardiomyocytes. Circulation research. 2014; 114:511-23.
2. Yang X, Rodriguez M, Pabon L, Fischer K A, Reinecke H, Regnier M, Sniadecki N J, Ruohola-Baker H and Murry C E. Tri-iodo-l-thyronine promotes the maturation of human cardiomyocytes-derived from induced pluripotent stem cells. Journal of molecular and cellular cardiology. 2014; 72:296-304.
3. Lundy S D, Zhu W Z, Regnier M and Laflamme M A. Structural and functional maturation of cardiomyocytes derived from human pluripotent stem cells. Stem cells and development. 2013; 22:1991-2002.
4. Sartiani L, Bettiol E, Stillitano F, Mugelli A, Cerbai E and Jaconi M E. Developmental changes in cardiomyocytes differentiated from human embryonic stem cells: a molecular and electrophysiological approach. Stem cells. 2007; 25:1136-44.
5. Yang X, Rodriguez M, Pabon L, Fischer K A, Reinecke H, Regnier M, Sniadecki N J, Baker H R and Murry C E. Tri-iodo-L-thyronine Promotes the Maturation of Cardiomyocytes-Derived from Human Induced Pluripotent Stem Cells Journal of molecular and cellular cardiology. 2014.
6. Kosmidis G, Bellin M, Ribeiro M C, van Meer B, Ward-van Oostwaard D, Passier R, Tertoolen L G, Mummery C L and Casini S. Altered calcium handling and increased contraction force in human embryonic stem cell derived cardiomyocytes following short term dexamethasone exposure. Biochemical and biophysical research communications. 2015.
7. Birket M J, Ribeiro M C, Kosmidis G, Ward D, Leitoguinho A R, van de Pol V, Dambrot C, Devalla H D, Davis R P, Mastroberardino P G, Atsma D E, Passier R and Mummery C L. Contractile Defect Caused by Mutation in MYBPC3 Revealed under Conditions Optimized for Human PSC-Cardiomyocyte Function. Cell reports. 2015; 13:733-45.
8. Hazeltine L B, Simmons C S, Salick M R, Lian X, Badur M G, Han W, Delgado S M, Wakatsuki T, Crone W C, Pruitt B L and Palecek S P. Effects of substrate mechanics on contractility of cardiomyocytes generated from human pluripotent stem cells. International journal of cell biology. 2012; 2012:508294.
9. Feaster T K, Cadar A G, Wang L, Williams C H, Chun Y W, Hempel J, Bloodworth N, Merryman W D, Lim C C, Wu J C, Knollmann B C and Hong C C. Matrigel Mattress: A Method for the Generation of Single Contracting Human-Induced Pluripotent Stem Cell-Derived Cardiomyocytes. Circulation research. 2015.
10. Lee D S, Chen J H, Lundy D J, Liu C H, Hwang S M, Pabon L, Shieh R C, Chen C C, Wu S N, Yan Y T, Lee S T, Chiang P M, Chien S, Murry C E and Hsieh P C. Defined MicroRNAs Induce Aspects of Maturation in Mouse and Human Embryonic-Stem-Cell-Derived Cardiomyocytes. Cell reports. 2015; 12:1960-7.
11. Kuppusamy K T, Jones D C, Sperber H, Madan A, Fischer K A, Rodriguez M L, Pabon L, Zhu W Z, Tulloch N L, Yang X, Sniadecki N J, Laflamme M A, Ruzzo W L, Murry C E and Ruohola-Baker H. Let-7 family of microRNA is required for maturation and adult-like metabolism in stem cell-derived cardiomyocytes. Proc Natl Acad Sci USA. 2015; 112:E2785-94.
12. Bedada F B, Chan S S, Metzger S K, Zhang L, Zhang J, Garry D J, Kamp T J, Kyba M and Metzger J M. Acquisition of a quantitative, stoichiometrically conserved ratiometric marker of maturation status in stem cell-derived cardiac myocytes. Stem cell reports. 2014; 3:594-605.
13. Tulloch N L, Muskheli V, Razumova M V, Korte F S, Regnier M, Hauch K D, Pabon L, Reinecke H and Murry C E. Growth of engineered human myocardium with mechanical loading and vascular coculture. Circulation research. 2011; 109:47-59.
14. Ruan J L, Tulloch N L, Saiget M, Paige S L, Razumova M V, Regnier M, Tung K C, Keller G, Pabon L, Reinecke H and Murry C E. Mechanical Stress Promotes Maturation of Human Myocardium From Pluripotent Stem Cell-Derived Progenitors. Stem Cells. 2015; 33:2148-57.
15. Makinde A O, Kantor P F and Lopaschuk G D. Maturation of fatty acid and carbohydrate metabolism in the newborn heart. Molecular and cellular biochemistry. 1998; 188:49-56.
16. Zaha V G and Young L H. AMP-activated protein kinase regulation and biological actions in the heart. Circulation research. 2012; 111:800-14.
17. Makinde A O, Gamble J and Lopaschuk G D. Upregulation of 5′-AMP-activated protein kinase is responsible for the increase in myocardial fatty acid oxidation rates following birth in the newborn rabbit. Circulation research. 1997; 80:482-9.
18. Hu X, Xu X, Lu Z, Zhang P, Fassett J, Zhang Y, Xin Y, Hall J L, Viollet B, Bache R J, Huang Y and Chen Y. AMP activated protein kinase-alpha2 regulates expression of estrogen-related receptor-alpha, a metabolic transcription factor related to heart failure development. Hypertension. 2011; 58:696-703.
19. Ventura-Clapier R, Gamier A and Veksler V. Transcriptional control of mitochondrial biogenesis: the central role of PGC-lalpha. Cardiovascular research. 2008; 79:208-17.
20. Terai K, Hiramoto Y, Masaki M, Sugiyama S, Kuroda T, Hori M, Kawase I and Hirota H. AMP-activated protein kinase protects cardiomyocytes against hypoxic injury through attenuation of endoplasmic reticulum stress. Molecular and cellular biology. 2005; 25:9554-75.
21. Stuck B J, Lenski M, Bohm M and Laufs U. Metabolic switch and hypertrophy of cardiomyocytes following treatment with angiotensin II are prevented by AMP-activated protein kinase. The Journal of biological chemistry. 2008; 283:32562-9.
22. Yu J, Vodyanik M A, Smuga-Otto K, Antosiewicz-Bourget J, Frane J L, Tian S, Nie J, Jonsdottir G A, Ruotti V, Stewart R, Slukvin, II and Thomson J A. Induced pluripotent stem cell lines derived from human somatic cells. Science. 2007; 318:1917-20.
23. Miyaoka Y, Chan A H, Judge L M, Yoo J, Huang M, Nguyen T D, Lizarraga P P, So P L and Conklin B R. Isolation of single-base genome-edited human iPS cells without antibiotic selection. Nature methods. 2014; 11:291-3.
24. Si-Tayeb K, Noto F K, Nagaoka M, Li J, Battle M A, Duris C, North P E, Dalton S and Duncan S A. Highly efficient generation of human hepatocyte-like cells from induced pluripotent stem cells. Hepatology. 2010; 51:297-305.
25. Mallanna S K and Duncan S A. Differentiation of hepatocytes from pluripotent stem cells. Curr Protoc Stem Cell Biol. 2013; 26:Unit 1G 4.
26. Yuan S H, Martin J, Elia J, Flippin J, Paramban R I, Hefferan M P, Vidal J G, Mu Y, Killian R L, Israel M A, Emre N, Marsala S, Marsala M, Gage F H, Goldstein L S and Carson C T. Cell-surface marker signatures for the isolation of neural stem cells, glia and neurons derived from human pluripotent stem cells. PloS one. 2011; 6:e17540.
27. Israel M A, Yuan S H, Bardy C, Reyna S M, Mu Y, Herrera C, Hefferan M P, Van Gorp S, Nazor K L, Boscolo F S, Carson C T, Laurent L C, Marsala M, Gage F H, Remes A M, Koo E H and Goldstein L S. Probing sporadic and familial Alzheimer's disease using induced pluripotent stem cells. Nature. 2012; 482:216-20.
28. Young J E, Boulanger-Weill J, Williams D A, WoodruffG, Buen F, Revilla A C, Herrera C, Israel M A, Yuan S H, Edland S D and Goldstein L S. Elucidating molecular phenotypes caused by the SORL1 Alzheimer's disease genetic risk factor using human induced pluripotent stem cells. Cell stem cell. 2015; 16:373-85.
29. Makarenko I, Opitz C A, Leake M C, Neagoe C, Kulke M, Gwathmey J K, del Monte F, Hajjar R J and Linke W A. Passive stiffness changes caused by upregulation of compliant titin isoforms in human dilated cardiomyopathy hearts. Circ Res. 2004; 95:708-16.
30. Rao M, Li L, Demello C, Guo D, Jaber B L, Pereira B J, Balakrishnan V S and Group H S. Mitochondrial DNA injury and mortality in hemodialysis patients. Journal of the American Society of Nephrology: JASN. 2009; 20:189-96.
31. Rodriguez A G, Han S J, Regnier M and Sniadecki N J. Substrate stiffness increases twitch power of neonatal cardiomyocytes in correlation with changes in myofibril structure and intracellular calcium. Biophysical journal. 2011; 101:2455-64.
32. Rodriguez M L, Graham B T, Pabon L M, Han S J, Murry C E and Sniadecki N J. Measuring the Contractile Forces of Human Induced Pluripotent Stem Cell-Derived Cardiomyocytes with Arrays of Microposts. J Biomechanical Engineering. 2014.
33. Tan J L, Tien J, Pirone D M, Gray D S, Bhadriraju K and Chen C S. Cells lying on a bed of microneedles: an approach to isolate mechanical force. Proceedings of the National Academy of Sciences of the United States of America. 2003; 100:1484-9.
34. Sniadecki N J and Chen C S. Microfabricated Silicone Elastomeric Post Arrays for Measuring Traction Forces of Adherent Cells. In: Y. Wang and D. E. Discher, eds. Methods in Cell Biology: Cell Mechanics San Diego, Calif.: Elsevier Inc.; 2007(83): 313-328.
35. Woods A, Azzout-Marniche D, Foretz M, Stein S C, Lemarchand P, Ferre P, Foufelle F and Carling D. Characterization of the role of AMP-activated protein kinase in the regulation of glucose-activated gene expression using constitutively active and dominant negative forms of the kinase. Molecular and cellular biology. 2000; 20:6704-11.
36. Foretz M, Ancellin N, Andreelli F, Saintillan Y, Grondin P, Kahn A, Thorens B, Vaulont S and Viollet B. Short-term overexpression of a constitutively active form of AMP-activated protein kinase in the liver leads to mild hypoglycemia and fatty liver. Diabetes. 2005; 54:1331-9.
37. Kim C, Wong J, Wen J, Wang S, Wang C, Spiering S, Kan N G, Forcales S, Puri P L, Leone T C, Marine J E, Calkins H, Kelly D P, Judge D P and Chen H S. Studying arrhythmogenic right ventricular dysplasia with patient-specific iPSCs. Nature. 2013; 494:105-10.
38. Lian X, Hsiao C, Wilson G, Zhu K, Hazeltine L B, Azarin S M, Raval K K, Zhang J, Kamp T J and Palecek S P. Robust cardiomyocyte differentiation from human pluripotent stem cells via temporal modulation of canonical Wnt signaling. Proc Natl Acad Sci USA. 2012; 109:E1848-57.
39. Anmann T, Varikmaa M, Timohhina N, Tepp K, Shevchuk I, Chekulayev V, Saks V and Kaambre T. Formation of highly organized intracellular structure and energy metabolism in cardiac muscle cells during postnatal development of rat heart. Biochimica et biophysica acta. 2014; 1837:1350-61.
40. Jacot J G, McCulloch A D and Omens J H. Substrate stiffness affects the functional maturation of neonatal rat ventricular myocytes. Biophysical journal. 2008; 95:3479-87.
41. Sasse S, Brand N J, Kyprianou P, Dhoot G K, Wade R, Arai M, Periasamy M, Yacoub M H and Barton P J. Troponin I gene expression during human cardiac development and in end-stage heart failure. Circulation research. 1993; 72:932-8.
42. Siedner S, Kruger M, Schroeter M, Metzler D, Roell W, Fleischmann B K, Hescheler J, Pfitzer G and Stehle R. Developmental changes in contractility and sarcomeric proteins from the early embryonic to the adult stage in the mouse heart. J Physiol. 2003; 548:493-505.
43. Corton J M, Gillespie J G, Hawley S A and Hardie D G. 5-aminoimidazole-4-carboxamide ribonucleoside. A specific method for activating AMP-activated protein kinase in intact cells? European journal of biochemistry/FEBS. 1995; 229:558-65.
44. Sajan M P, Bandyopadhyay G, Miura A, Standaert M L, Nimal S, Longnus S L, Van Obberghen E, Hainault I, Foufelle F, Kahn R, Braun U, Leitges M and Farese R V. AICAR and metformin, but not exercise, increase muscle glucose transport through AMPK-, ERK-, and PDK1-dependent activation of atypical PKC. American journal of physiology Endocrinology and metabolism. 2010; 298:E179-92.
45. Kim M and Tian R. Targeting AMPK for cardiac protection: opportunities and challenges. Journal of molecular and cellular cardiology. 2011; 51:548-53.
46. Zheng X, Boyer L, Jin M, Mertens J, Kim Y, Ma L, Ma L, Hamm M, Gage F H and Hunter T. Metabolic reprogramming during neuronal differentiation from aerobic glycolysis to neuronal oxidative phosphorylation. Elife. 2016; 5.
47. Samat H B. Immunocytochemical markers of neuronal maturation in human diagnostic neuropathology. Cell Tissue Res. 2015; 359:279-94.
48. Poon A, Zhang Y, Chandrasekaran A, Phanthong P, Schmid B, Nielsen T T and Freude K K. Modeling neurodegenerative diseases with patient-derived induced pluripotent cells: Possibilities and challenges. N Biotechnol. 2017.
49. Muratore C R, Rice H C, Srikanth P, Callahan D G, Shin T, Benjamin L N, Walsh D M, Selkoe D J and Young-Pearse T L. The familial Alzheimer's disease APPV717I mutation alters APP processing and Tau expression in iPSC-derived neurons. Human molecular genetics. 2014; 23:3523-36.
50. Yagi T, Ito D, Okada Y, Akamatsu W, Nihei Y, Yoshizaki T, Yamanaka S, Okano H and Suzuki N. Modeling familial Alzheimers disease with induced pluripotent stem cells. Human molecular genetics. 2011; 20:4530-9.
51. Cabezas-Opazo F A, Vergara-Pulgar K, Perez M J, Jara C, Osorio-Fuentealba C and Quintanilla R A. Mitochondrial Dysfunction Contributes to the Pathogenesis of Alzheimers Disease. Oxid Med Cell Longev. 2015; 2015:509654.
52. Salminen A, Haapasalo A, Kauppinen A, Kaarniranta K, Soininen H and Hiltunen M. Impaired mitochondrial energy metabolism in Alzheimer's disease: Impact on pathogenesis via disturbed epigenetic regulation of chromatin landscape. Prog Neurobiol. 2015; 131:1-20.
53. Rovelet-Lecrux A, Hannequin D, Raux G, Le Meur N, Laquerriere A, Vital A, Dumanchin C, Feuillette S, Brice A, Vercelletto M, Dubas F, Frebourg T and Campion D. APP locus duplication causes autosomal dominant early-onset Alzheimer disease with cerebral amyloid angiopathy. Nat Genet. 2006; 38:24-6.
54. Grattagliano I, Russmann S, Diogo C, Bonfrate L, Oliveira P J, Wang D Q and Portincasa P. Mitochondria in chronic liver disease. Curr Drug Targets. 2011; 12:879-93.
55. Finck B N and Kelly D P. PGC-1 coactivators: inducible regulators of energy metabolism in health and disease. The Journal of clinical investigation. 2006; 116:615-22.
56. Grant D A, Fauchere J C, Eede K J, Tyberg J V and Walker A M. Left ventricular stroke volume in the fetal sheep is limited by extracardiac constraint and arterial pressure. The Journal of physiology. 2001; 535:231-9.
57. Guo W, Schafer S, Greaser M L, Radke M H, Liss M, Govindarajan T, Maatz H, Schulz H, Li S, Parrish A M, Dauksaite V, Vakeel P, Klaassen S, Gerull B, Thierfelder L, Regitz-Zagrosek V, Hacker T A, Saupe K W, Dec G W, Ellinor P T, MacRae C A, Spallek B, Fischer R, Perrot A, Ozcelik C, Saar K, Hubner N and Gotthardt M. RBM20, a gene for hereditary cardiomyopathy, regulates titin splicing. Nature medicine. 2012; 18:766-73.
58. Kruger M, Sachse C, Zimmermann W H, Eschenhagen T, Klede S and Linke W A. Thyroid hormone regulates developmental titin isoform transitions via the phosphatidylinositol-3-kinase/AKT pathway. Circ Res. 2008; 102:439-47.
59. Hahn-Windgassen A, Nogueira V, Chen C C, Skeen J E, Sonenberg N and Hay N. Akt activates the mammalian target of rapamycin by regulating cellular ATP level and AMPK activity. The Journal of biological chemistry. 2005; 280:32081-9.
60. Yoon M J, Lee G Y, Chung J J, Ahn Y H, Hong S H and Kim J B. Adiponectin increases fatty acid oxidation in skeletal muscle cells by sequential activation of AMP-activated protein kinase, p38 mitogen-activated protein kinase, and peroxisome proliferator-activated receptor alpha. Diabetes. 2006; 55:2562-70.
61. Barger P M, Browning A C, Gamer A N and Kelly D P. p38 mitogen-activated protein kinase activates peroxisome proliferator-activated receptor alpha: a potential role in the cardiac metabolic stress response. The Journal of biological chemistry. 2001; 276:44495-501.
62. Rog-Zielinska E A, Craig M A, Manning J R, Richardson R V, Gowans G J, Dunbar D R, Gharbi K, Kenyon C J, Holmes M C, Hardie D G, Smith G L and Chapman K E. Glucocorticoids promote structural and functional maturation of foetal cardiomyocytes: a role for PGC-lalpha. Cell death and differentiation. 2014.
63. Montessuit C, Palma T, Viglino C, Pellieux C and Lerch R. Effects of insulin-like growth factor-I on the maturation of metabolism in neonatal rat cardiomyocytes. Pflugers Archiv: European journal of physiology. 2006; 452:380-6.
64. Ito H, Hiroe M, Hirata Y, Tsujino M, Adachi S, Shichiri M, Koike A, Nogami A and Marumo F. Insulin-like growth factor-I induces hypertrophy with enhanced expression of muscle specific genes in cultured rat cardiomyocytes. Circulation. 1993; 87:1715-21.
65. Radisic M, Park H, Shing H, Consi T, Schoen F J, Langer R, Freed L E and Vunjak-Novakovic G. Functional assembly of engineered myocardium by electrical stimulation of cardiac myocytes cultured on scaffolds. Proceedings of the National Academy of Sciences of the United States of America. 2004; 101:18129-34.
66. Xia Y, Buja L M, Scarpulla R C and McMillin J B. Electrical stimulation of neonatal cardiomyocytes results in the sequential activation of nuclear genes governing mitochondrial proliferation and differentiation. Proceedings of the National Academy of Sciences of the United States of America. 1997; 94:11399-404.
67. Chan Y C, Ting S, Lee Y K, Ng K M, Zhang J, Chen Z, Siu C W, Oh S K and Tse H F. Electrical stimulation promotes maturation of cardiomyocytes derived from human embryonic stem cells. Journal of cardiovascular translational research. 2013; 6:989-99.
68. Lasher R A, Pahnke A Q, Johnson J M, Sachse F B and Hitchcock R W. Electrical stimulation directs engineered cardiac tissue to an age-matched native phenotype. Journal of tissue engineering. 2012; 3:2041731412455354.
69. Fu J D, Rushing S N, Lieu D K, Chan C W, Kong C W, Geng L, Wilson K D, Chiamvimonvat N, Boheler K R, Wu J C, Keller G, Hajjar R J and Li R A. Distinct roles of microRNA-1 and -499 in ventricular specification and functional maturation of human embryonic stem cell-derived cardiomyocytes. PloS one. 2011; 6:e27417.
70. Callis T E, Pandya K, Seok H Y, Tang R H, Tatsuguchi M, Huang Z P, Chen J F, Deng Z, Gunn B, Shumate J, Willis M S, Selzman C H and Wang D Z. MicroRNA-208a is a regulator of cardiac hypertrophy and conduction in mice. The Journal of clinical investigation. 2009; 119:2772-86.
71. van Rooij E, Sutherland L B, Qi X, Richardson J A, Hill J and Olson E N. Control of stress-dependent cardiac growth and gene expression by a microRNA. Science. 2007; 316:575-9.
72. Odawara A, Katoh H, Matsuda N and Suzuki I. Physiological maturation and drug responses of human induced pluripotent stem cell-derived cortical neuronal networks in long-term culture. Sci Rep. 2016; 6:26181.
73. Tang X, Zhou L, Wagner A M, Marchetto M C, Muotri A R, Gage F H and Chen G. Astroglial cells regulate the developmental timeline of human neurons differentiated from induced pluripotent stem cells. Stem cell research. 2013; 11:743-57.
74. Cheng A, Hou Y and Mattson M P. Mitochondria and neuroplasticity. ASN Neuro. 2010; 2:e00045.
75. Posner H, Curiel R, Edgar C, Hendrix S, Liu E, Loewenstein D A, Morrison G, Shinobu L, Wesnes K and Harvey P D. Outcomes Assessment in Clinical Trials of Alzheimer's Disease and its Precursors: Readying for Short-term and Long-term Clinical Trial Needs. Innov Clin Neurosci. 2017; 14:22-29.
76. Brownjohn P W, Smith J, Portelius E, Semeels L, Kvartsberg H, De Strooper B, Blennow K, Zetterberg H and Livesey F J. Phenotypic Screening Identifies Modulators of Amyloid Precursor Protein Processing in Human Stem Cell Models of Alzheimer's Disease. Stem cell reports. 2017; 8:870-882.

| SEQUENCES | |
|---|---|
| (5′-AMP-activated protein kinase catalytic subunit alpha-1 isoform 1 | |
| [Homo sapiens]-NP_006242.5) | |
| SEQ ID NO: 1 | |
| 1 | mrrlsswrkm ataekqkhdg rvkighyilg dtlgvgtfgk vkvgkheltg hkvavkilnr |
| 61 | qkirsldvvg kirreignlk lfrhphiikl yqvistpsdi fmvmeyvsgg elfdyickng |
| 121 | rldekesrrl fqqilsgvdy chrhmvvhrd lkpenvllda hmnakiadfg lsnmmsdgef |
| 181 | lrtscgspny aapevisgrl yagpevdiws sgvilyallc gtlpfdddhv ptlfkkicdg |
| 241 | ifytpqylnp svisllkhml qvdpmkrati kdirehewfk qdlpkylfpe dpsysstmid |
| 301 | dealkevcek fecseeevls clynrnhqdp lavayhliid nrrimneakd fylatsppds |
| 361 | flddhhltrp hpervpflva etprarhtld elnpqkskhq gvrkakwhlg irsqsrpndi |
| 421 | maevcraikq ldyewkvvnp yylrvrrknp vtstyskmsl qlyqvdsrty lldfrsidde |
| 481 | iteaksgtat pqrsgsysny rscqrsdsda eaqgkssevs ltssvtslds spvdltprpg |
| 541 | shtieffemc anlikilaq |
| (Homo sapiens protein kinase AMP-activated catalytic subunit alpha 1 | |
| (PRKAA1), transcript variant 1, mRNA- NM_006251.5) | |
| SEQ ID NO: 2 | |
| 1 | agcgccatgc gcagactcag ttcctggaga aagatggcga cagccgagaa gcagaaacac |
| 61 | gacgggcggg tgaagatcgg ccactacatt ctgggtgaca cgctgggggt cggcaccttc |
| 121 | ggcaaagtga aggttggcaa acatgaattg actgggcata aagtagctgt gaagatactc |
| 181 | aatcgacaga agattcggag ccttgatgtg gtaggaaaaa tccgcagaga aattcagaac |
| 241 | ctcaagcttt tcaggcatcc tcatataatt aaactgtacc aggtcatcag tacaccatct |
| 301 | gatattttca tggtgatgga atatgtctca ggaggagagc tatttgatta tatctgtaag |
| 361 | aatggaaggc tggatgaaaa agaaagtcgg cgtctgttcc aacagatcct ttctggtgtg |
| 421 | gattattgtc acaggcatat ggtggtccat agagatttga aacctgaaaa tgtcctgctt |
| 481 | gatgcacaca tgaatgcaaa gatagctgat tttggtcttt caaacatgat gtcagatggt |
| 541 | gaatttttaa gaacaagttg tggctcaccc aactatgctg caccagaagt aatttcagga |
| 601 | agattgtatg caggcccaga ggtagatata tggagcagtg gggttattct ctatgcttta |
| 661 | ttatgtggaa cccttccatt tgatgatgac catgtgccaa ctctttttaa gaagatatgt |
| 721 | gatgggatct tctatacccc tcaatattta aatccttctg tgattagcct tttgaaacat |
| 781 | atgctgcagg tggatcccat gaagagggcc acaatcaaag atatcaggga acatgaatgg |
| 841 | tttaaacagg accttccaaa atatctcttt cctgaggatc catcatatag ttcaaccatg |
| 901 | attgatgatg aagccttaaa agaagtatgt gaaaagtttg agtgctcaga agaggaagtt |
| 961 | ctcagctgtc tttacaacag aaatcaccag gatcctttgg cagttgccta ccatctcata |
| 1021 | atagataaca ggagaataat gaatgaagcc aaagatttct atttggcgac aagcccacct |
| 1081 | gattcttttc ttgatgatca tcacctgact cggccccatc ctgaaagagt accattcttg |
| 1141 | gttgctgaaa caccaagggc acgccatacc cttgatgaat taaatccaca gaaatccaaa |
| 1201 | caccaaggtg taaggaaagc aaaatggcat ttaggaatta gaagtcaaag tcgaccaaat |
| 1261 | gatattatgg cagaagtatg tagagcaatc aaacaattgg attatgaatg gaaggttgta |
| 1321 | aacccatatt atttgcgtgt acgaaggaag aatcctgtga caagcactta ctccaaaatg |
| 1381 | agtctacagt tataccaagt ggatagtaga acttatctac tggatttccg tagtattgat |
| 1441 | gatgaaatta cagaagccaa atcagggact gctactccac agagatcggg atcagttagc |
| 1501 | aactatcgat cttgccaaag gagtgattca gatgctgagg ctcaaggaaa atcctcagaa |
| 1561 | gtttctctta cctcatctgt gacctcactt gactcttctc ctgttgacct aactccaaga |
| 1621 | cctggaagtc acacaataga attttttgag atgtgtgcaa atctaattaa aattcttgca |
| 1681 | caataaacag aaaactttgc ttatttcttt tgcagcaata agcatgcata ataagtcaca |
| 1741 | gccaaatgct tccatttgta atcaagttat acataattat aaccgagggc tggcgttttg |
| 1801 | gaatgcaatt tgcacaggga ttggaacatg atttatagtt aaaagcctaa tatgcagaaa |
| 1861 | tgaattaaga tcattttgtt gttcattgtg cagtatgtat atagcataat atacacagtg |
| 1921 | aattataggt ctcaggctta cttgattttt ggctatttta tatttagtgt acacagggct |
| 1981 | ttgaaatatt aatttacata aaggccttca tatattatta cgtgttatat attacgtgtt |
| 2041 | ataaatttat tcaataaata tttgcctaga attcccaaga cctttatagg tgattttgtt |
| 2101 | ttctgggctc cttaacttca taaatagcta gtatcttcca gcagtagtaa cagtctggat |
| 2161 | aacttcttcc atatccctcc ctctttgttt ttttgagaca gtgtcacttt gtcacccagg |
| 2221 | ctggagtgca atggtgtggt ctcggctcac tgcaacctcc acctcccggg ttcaagtgat |
| 2281 | tctcccgcct cagcttcctg agtagctgga actacaggcg tgtgccacca cacccggcta |
| 2341 | atttttcgta tttttagtgt agacggggtt tcactatgtt gcccaggctg gtctcgaact |
| 2401 | cctgaccgcg tgatccacca cctcagcttc ccaaagtggt gggattacag gcgtgagcca |
| 2461 | ccgcacccgg cctccatatc ccccttttaa aattctgtag tgtatggtaa gtcatatcag |
| 2521 | atatcagacc taatttaaat ttcattttag ctttacaagt ccaaaaacac agaatttata |
| 2581 | tattcagata ctctagcact aattttagtc ttaaaatatt cccacgatat tctgtacaca |
| 2641 | aaatgttctt tttgttacaa gagctgagtt gcatatactg tagataaatc atattatttt |
| 2701 | tgccaatttc acaaattcct ctggcccatc atgtcagtca ttattgagta tatgcacaca |
| 2761 | ttgctactta tttgattatg tatcttttaa attgattcag tgcatagaaa actatctctt |
| 2821 | acaaacttta agtgctctga tatgacttcc cccccaaatt ttattatgaa catttttaaa |
| 2881 | aacagaaaaa ttgaaaaact gtttggtaag cacatgtata tctaccattt agattcagca |
| 2941 | gttgttaatg ttttgtcatt tgttttctct atacctatat atgtatagat acagctagtt |
| 3001 | atgcatatat atgcatatat gtgtttgttt gtgtatgtat atatgctttt ttccccctga |
| 3061 | accatttgga tgttacagac atacttatca ccgtgaaaat acttcaagta tctcctacag |
| 3121 | ataatgacat tctcctaaaa atccgtaata ccattgtaaa agtaataatt ccccaatatc |
| 3181 | atctaatcaa gccatattta aatttctgaa gttaactcca aatttcttta tagctgatta |
| 3241 | tttcaaacta ggatccaatt aaagtttaca tatgacactt ggttataact ctttagttgg |
| 3301 | atataacatt attattattt tgataaaata tggaacaaat caattctatt aataagtggt |
| 3361 | cacatttgtt ttgggcttaa attacttttt aaagatactg gattttccta agatttctga |
| 3421 | tttacactga tatttttttt tgtcattctt aattgcatca cacaatagat gtaaatgaag |
| 3481 | atgtagtcac ctcagataaa attggtatcg tgtatgataa tattgtatca tttatatttg |
| 3541 | ccttatgtta actttaagaa attgattttt ttgtattaat cattttccca ttgcaacaga |
| 3601 | gctatatttt ttctatttta agaatcatat tttaggatta tttttggcaa atacagtgag |
| 3661 | cacttatgta accagatgat aatgaactca aatgtcatga tagcttgcat aaatggtgac |
| 3721 | tctagtagat ttgactcaag cacttctaga atcatgcact gaattcaaaa gaaaaatctt |
| 3781 | gctgcttttt gtccagggct tgttctattc aacttctaat ttgaaagctg tacaaagtaa |
| 3841 | tagaagttcc atttaaatat gagttcaaaa ctgtatttac tttttatgtg gccctctctt |
| 3901 | taggggattc taattttact tagggtctct aagtgcagca taatgttcct gatgttaaca |
| 3961 | gaagactgta tttttaaagt tacaaatttg tatatggaat taagtaatgg cgctatatac |
| 4021 | gctgttgtgg ggagggggga agaaaaggag gaaccaatta aataggacct tttaaaaatt |
| 4081 | gttaattttg taaactttgc ttctcttata agttattgtg attcatttta gttactgtgt |
| 4141 | tttattttga aaatatttaa atattgcact tctataaata gtatgataaa tgcacagaca |
| 4201 | attgcagtaa attctttttt aagctaggat atttgaaatg acaacctttg gttaagtgtg |
| 4261 | tcaaggttgc aacagaattt tcacaatttt tttgttgttt gcaaattgtt actaatattg |
| 4321 | aagaggtaag ggaggcaatg caaatgattt ttaatctttt tttattatct tttcagcagt |
| 4381 | ttatattttt tgtgacttta tgcaaccata tttttacttt gtcttgacaa ctgaaagatg |
| 4441 | tataaggttt tttgccagaa atgtactgta tacatagttt taagtataac agattttact |
| 4501 | gatatgtaaa aattttgcca ttaaaataaa tgatttctca ctgagaggaa cttttctacc |
| 4561 | aggttggggc atatgggagc ttaatatatc atatctaatt taaaataatt tcactgaaat |
| 4621 | aaactccatt gcttttacct aatttttttc ttgagatgct tttgtagttt ttcagagttt |
| 4681 | tagatgattt tatacaaaat cctctgccta gcactgctct ttttgatgtt gtagtgacac |
| 4741 | catttacatt gaattaatgc ttggtagcct ggggctagat gtggaactcc atggatctgt |
| 4801 | gttctgactg gcacctttgg aatgaaagaa aagtgtgtgc tgtccaaatt ttttcccctt |
| 4861 | aattctttcc ctcatcttct cacccataat agaaatttta tttccattgt gagttctgac |
| 4921 | aagaatgaaa ttccacatac aacataactg taaattgttg gtaggtagaa gttaatattt |
| 4981 | gtggttcatg tatattttga ccagagtata tttaagtata taatttcagc ttccttgatt |
| 5041 | tagaaatatg atataataaa gaaaaactcc atttatcatc tgtta |

1. A method of promoting maturation of in vitro-differentiated cardiomyocytes, the method comprising treating in vitro-differentiated cardiomyocytes with an activator of adenosine monophosphate-activated protein kinase (AMPK).

2. The method of claim 1, wherein the activator of AMPK comprises a small molecule, a polypeptide, a nucleic acid encoding a polypeptide or a vector encoding a polypeptide.

3. The method of claim 2, wherein the small molecule is 5-aminoimidizole-4-carboxamide riboside (AICAR) or a derivative thereof that activates AMPK.

4. The method of claim 3, wherein the derivative is 5-aminoimidazole-4-carboxamide-1-β-D-ribofuranosyl-5′-monophosphate (ZMP).

5. The method of claim 2, wherein the polypeptide comprises AMPK.

6. The method of claim 1, wherein the activator comprises a vector encoding an AMPK polypeptide.

7. The method of claim 2, wherein the AMPK polypeptide is a constitutively active polypeptide.

8. The method of claim 2, wherein the nucleic acid encoding the polypeptide or the vector that encodes the polypeptide permits inducible expression of the polypeptide.

9. The method of claim 2, wherein the vector is selected from the group consisting of: a lentiviral vector, an adenoviral vector, an adeno-associated virus vector (AAV), episomal vector, an EBNA1 vector, a minicircle vector, and a Sendai virus vector.

10. The method of claim 1, wherein the in vitro differentiated cardiomyocytes are human.

11. The method of claim 1, wherein the in vitro differentiated cardiomyocytes are differentiated from induced pluripotent stem cells (iPSCs) or from embryonic stem cells.

12. The method of claim 1, wherein the in vitro differentiated cardiomyocytes are derived from a subject having a cardiac disease or disorder.

13. The method of claim 12, wherein the cardiac disease or disorder is selected from the group consisting of: arrhythmogenic right ventricular dysplasia (ARVD), cardiomyopathy, cardiac arrhythmia, cardiomyopathy, long QT syndrome, catecholaminergic polymorphic ventricular tachycardia (CPVT), Barth syndrome, and Duchenne muscular dystrophy-related cardiac disease.

14. The method of claim 1, wherein treatment with an activator of AMPK promotes one or more of electrical maturity, metabolic maturity, and/or contractile maturity of in vitro-differentiated cardiomyocytes.

15. The method of claim 14, wherein electrical maturity is determined by one or more of the following markers as compared to a reference level: increased gene expression of an ion channel gene, increased sodium current density, increased inwardly-rectifying potassium channel current density, decreased action potential frequency, decreased calcium wave frequency, and decreased field potential frequency.

16. (canceled)

17. The method of claim 14, wherein contractile maturity is determined by one or more of the following markers as compared to a reference level: decreased beat frequency, increased contractile force, increased level or activity of α-myosin heavy chain (α-MHC), increased level or activity of sarcomeres, decreased circularity index, increased level or activity of troponin, increased level or activity of titin N2b, increased cell area, and increased aspect ratio.

18. The method of claim 1, further comprising contacting the in vitro-differentiated cardiomyocytes with a nanopatterned substrate.

19. A method of transplanting in vitro-differentiated cardiomyocytes in a subject, the method comprising:
(a) contacting in vitro-differentiated cardiomyocytes with an activator of AMPK; and
(b) transplanting said in vitro-differentiated cardiomyocytes into the subject.

20.-39. (canceled)

40. A method of evaluating toxicity of an agent, the method comprising contacting in vitro-differentiated cardiomyocytes or neurons prepared by the method of claim 1, respectively, with an agent.

41.-43. (canceled)

44. A composition comprising in vitro-differentiated cardiomyocytes made by contacting in vitro-differentiated cardiomyocytes with an activator of adenosine monophosphate-activated protein kinase (AMPK), wherein the cardiomyocytes have a more mature phenotype as compared with in vitro-differentiated cardiomyocytes that were not contacted with an activator of adenosine monophosphate-activated protein kinase (AMPK).

45.-53. (canceled)


SEQUENCE LISTING (75 sequences)

SEQ ID NO: 1 - protein, 559 aa, Homo sapiens
MRRLSSWRKMATAEKQKHDGRVKIGHYILGDTLGVGTFGKVKVGKHELTGHKVAVKILNRQKIRSLDVVGKIRREIQNLKLFRHPHIIKLYQVISTPSDIFMVMEYVSGGELFDYICKNGRLDEKESRRLFQQILSGVDYCHRHMVVHRDLKPENVLLDAHMNAKIADFGLSNMMSDGEFLRTSCGSPNYAAPEVISGRLYAGPEVDIWSSGVILYALLCGTLPFDDDHVPTLFKKICDGIFYTPQYLNPSVISLLKHMLQVDPMKRATIKDIREHEWFKQDLPKYLFPEDPSYSSTMIDDEALKEVCEKFECSEEEVLSCLYNRNHQDPLAVAYHLIIDNRRIMNEAKDFYLATSPPDSFLDDHHLTRPHPERVPFLVAETPRARHTLDELNPQKSKHQGVRKAKWHLGIRSQSRPNDIMAEVCRAIKQLDYEWKVVNPYYLRVRRKNPVTSTYSKMSLQLYQVDSRTYLLDFRSIDDEITEAKSGTATPQRSGSVSNYRSCQRSDSDAEAQGKSSEVSLTSSVTSLDSSPVDLTPRPGSHTIEFFEMCANLIKILAQ

SEQ ID NO: 2 - DNA, 5085 bp, Homo sapiens
agcgccatgcgcagactcagttcctggagaaagatggcgacagccgagaagcagaaacacgacgggcgggtgaagatcggccactacattctgggtgacacgctgggggtcggcaccttcggcaaagtgaaggttggcaaacatgaattgactgggcataaagtagctgtgaagatactcaatcgacagaagattcggagccttgatgtggtaggaaaaatccgcagagaaattcagaacctcaagcttttcaggcatcctcatataattaaactgtaccaggtcatcagtacaccatctgatattttcatggtgatggaatatgtctcaggaggagagctatttgattatatctgtaagaatggaaggctggatgaaaaagaaagtcggcgtctgttccaacagatcctttctggtgtggattattgtcacaggcatatggtggtccatagagatttgaaacctgaaaatgtcctgcttgatgcacacatgaatgcaaagatagctgattttggtctttcaaacatgatgtcagatggtgaatttttaagaacaagttgtggctcacccaactatgctgcaccagaagtaatttcaggaagattgtatgcaggcccagaggtagatatatggagcagtggggttattctctatgctttattatgtggaacccttccatttgatgatgaccatgtgccaactctttttaagaagatatgtgatgggatcttctatacccctcaatatttaaatccttctgtgattagccttttgaaacatatgctgcaggtggatcccatgaagagggccacaatcaaagatatcagggaacatgaatggtttaaacaggaccttccaaaatatctctttcctgaggatccatcatatagttcaaccatgattgatgatgaagccttaaaagaagtatgtgaaaagtttgagtgctcagaagaggaagttctcagctgtctttacaacagaaatcaccaggatcctttggcagttgcctaccatctcataatagataacaggagaataatgaatgaagccaaagatttctatttggcgacaagcccacctgattcttttcttgatgatcatcacctgactcggccccatcctgaaagagtaccattcttggttgctgaaacaccaagggcacgccatacccttgatgaattaaatccacagaaatccaaacaccaaggtgtaaggaaagcaaaatggcatttaggaattagaagtcaaagtcgaccaaatgatattatggcagaagtatgtagagcaatcaaacaattggattatgaatggaaggttgtaaacccatattatttgcgtgtacgaaggaagaatcctgtgacaagcacttactccaaaatgagtctacagttataccaagtggatagtagaacttatctactggatttccgtagtattgatgatgaaattacagaagccaaatcagggactgctactccacagagatcgggatcagttagcaactatcgatcttgccaaaggagtgattcagatgctgaggctcaaggaaaatcctcagaagtttctcttacctcatctgtgacctcacttgactcttctcctgttgacctaactccaagacctggaagtcacacaatagaattttttgagatgtgtgcaaatctaattaaaattcttgcacaataaacagaaaactttgcttatttcttttgcagcaataagcatgcataataagtcacagccaaatgcttccatttgtaatcaagttatacataattataaccgagggctggcgttttggaatgcaatttgcacagggattggaacatgatttatagttaaaagcctaatatgcagaaatgaattaagatcattttgttgttcattgtgcagtatgtatatagcataatatacacagtgaattataggtctcaggcttacttgatttttggctattttatatttagtgtacacagggctttgaaatattaatttacataaaggccttcatatattattacgtgttatatattacgtgttataaatttattcaataaatatttgcctagaattcccaagacctttataggtgattttgttttctgggctccttaacttcataaatagctagtatcttccagcagtagtaacagtctggataacttcttccatatccctccctctttgtttttttgagacagtgtcactttgtcacccaggctggagtgcaatggtgtggtctcggctcactgcaacctccacctcccgggttcaagtgattctcccgcctcagcttcctgagtagctggaactacaggcgtgtgccaccacacccggctaatttttcgtatttttagtgtagacggggtttcactatgttgcccaggctggtctcgaactcctgaccgcgtgatccaccacctcagcttcccaaagtggtgggattacaggcgtgagccaccgcacccggcctccatatcccccttttaaaattctgtagtgtatggtaagtcatatcagatatcagacctaatttaaatttcattttagctttacaagtccaaaaacacagaatttatatattcagatactctagcactaattttagtcttaaaatattcccacgatattctgtacacaaaatgttctttttgttacaagagctgagttgcatatactgtagataaatcatattatttttgccaatttcacaaattcctctggcccatcatgtcagtcattattgagtatatgcacacattgctacttatttgattatgtatcttttaaattgattcagtgcatagaaaactatctcttacaaactttaagtgctctgatatgacttcccccccaaattttattatgaacatttttaaaaacagaaaaattgaaaaactgtttggtaagcacatgtatatctaccatttagattcagcagttgttaatgttttgtcatttgttttctctatacctatatatgtatagatacagctagttatgcatatatatgcatatatgtgtttgtttgtgtatgtatatatgcttttttccccctgaaccatttggatgttacagacatacttatcaccgtgaaaatacttcaagtatctcctacagataatgacattctcctaaaaatccgtaataccattgtaaaagtaataattccccaatatcatctaatcaagccatatttaaatttctgaagttaactccaaatttctttatagctgattatttcaaactaggatccaattaaagtttacatatgacacttggttataactctttagttggatataacattattattattttgataaaatatggaacaaatcaattctattaataagtggtcacatttgttttgggcttaaattactttttaaagatactggattttcctaagatttctgatttacactgatatttttttttgtcattcttaattgcatcacacaatagatgtaaatgaagatgtagtcacctcagataaaattggtatcgtgtatgataatattgtatcatttatatttgccttatgttaactttaagaaattgatttttttgtattaatcattttcccattgcaacagagctatattttttctattttaagaatcatattttaggattatttttggcaaatacagtgagcacttatgtaaccagatgataatgaactcaaatgtcatgatagcttgcataaatggtgactctagtagatttgactcaagcacttctagaatcatgcactgaattcaaaagaaaaatcttgctgctttttgtccagggcttgttctattcaacttctaatttgaaagctgtacaaagtaatagaagttccatttaaatatgagttcaaaactgtatttactttttatgtggccctctctttaggggattctaattttacttagggtctctaagtgcagcataatgttcctgatgttaacagaagactgtatttttaaagttacaaatttgtatatggaattaagtaatggcgctatatacgctgttgtggggaggggggaagaaaaggaggaaccaattaaataggaccttttaaaaattgttaattttgtaaactttgcttctcttataagttattgtgattcattttagttactgtgttttattttgaaaatatttaaatattgcacttctataaatagtatgataaatgcacagacaattgcagtaaattcttttttaagctaggatatttgaaatgacaacctttggttaagtgtgtcaaggttgcaacagaattttcacaatttttttgttgtttgcaaattgttactaatattgaagaggtaagggaggcaatgcaaatgatttttaatctttttttattatcttttcagcagtttatattttttgtgactttatgcaaccatatttttactttgtcttgacaactgaaagatgtataaggttttttgccagaaatgtactgtatacatagttttaagtataacagattttactgatatgtaaaaattttgccattaaaataaatgatttctcactgagaggaacttttctaccaggttggggcatatgggagcttaatatatcatatctaatttaaaataatttcactgaaataaactccattgcttttacctaatttttttcttgagatgcttttgtagtttttcagagttttagatgattttatacaaaatcctctgcctagcactgctctttttgatgttgtagtgacaccatttacattgaattaatgcttggtagcctggggctagatgtggaactccatggatctgtgttctgactggcacctttggaatgaaagaaaagtgtgtgctgtccaaattttttccccttaattctttccctcatcttctcacccataatagaaattttatttccattgtgagttctgacaagaatgaaattccacatacaacataactgtaaattgttggtaggtagaagttaatatttgtggttcatgtatattttgaccagagtatatttaagtatataatttcagcttccttgatttagaaatatgatataataaagaaaaactccatttatcatctgtta

SEQ ID NO: 3 - DNA, 21 bp, Artificial Sequence
  Description of Artificial Sequence Synthetic primer
tgacactggcaaaacaatgca

SEQ ID NO: 4 - DNA, 20 bp, Artificial Sequence
  Description of Artificial Sequence Synthetic primer
caagttggaagacgagtgct

SEQ ID NO: 5 - DNA, 20 bp, Artificial Sequence
  Description of Artificial Sequence Synthetic primer
cttcggagaagcctaggcgc

SEQ ID NO: 6 - DNA, 20 bp, Artificial Sequence
  Description of Artificial Sequence Synthetic primer
agctctgtcacgatttgagg

SEQ ID NO: 7 - DNA, 20 bp, Artificial Sequence
  Description of Artificial Sequence Synthetic primer
cctctgtgacctctttgacc

SEQ ID NO: 8 - DNA, 21 bp, Artificial Sequence
  Description of Artificial Sequence Synthetic primer
attacggagtccacgcgtgtg

SEQ ID NO: 9 - DNA, 21 bp, Artificial Sequence
  Description of Artificial Sequence Synthetic primer
cagaacaaggaggcggaggtc

SEQ ID NO: 10 - DNA, 20 bp, Artificial Sequence
  Description of Artificial Sequence Synthetic primer
cgctcccccttcagttttgt

SEQ ID NO: 11 - DNA, 20 bp, Artificial Sequence
  Description of Artificial Sequence Synthetic primer
ccgaggtggttttcatctgt

SEQ ID NO: 12 - DNA, 20 bp, Artificial Sequence
  Description of Artificial Sequence Synthetic primer
tctgagagggccaagcaaag

SEQ ID NO: 13 - DNA, 20 bp, Artificial Sequence
  Description of Artificial Sequence Synthetic primer
gtcaccacccaaatccttat

SEQ ID NO: 14 - DNA, 19 bp, Artificial Sequence
  Description of Artificial Sequence Synthetic primer
ccgattggcagagtagtag

SEQ ID NO: 15 - DNA, 20 bp, Artificial Sequence
  Description of Artificial Sequence Synthetic primer
agaggtggagcatttctcgc

SEQ ID NO: 16 - DNA, 22 bp, Artificial Sequence
  Description of Artificial Sequence Synthetic primer
gtaaaaagagctgccccagtga

SEQ ID NO: 17 - DNA, 19 bp, Artificial Sequence
  Description of Artificial Sequence Synthetic primer
cagcagaactcagaatcga

SEQ ID NO: 18 - DNA, 21 bp, Artificial Sequence
  Description of Artificial Sequence Synthetic primer
ccaatgagtatggcagtgtca

SEQ ID NO: 19 - DNA, 23 bp, Artificial Sequence
  Description of Artificial Sequence Synthetic primer
cccagctccacgaggactgaaca

SEQ ID NO: 20 - DNA, 20 bp, Artificial Sequence
  Description of Artificial Sequence Synthetic primer
ggaacctcgccctgcaccag

SEQ ID NO: 21 - DNA, 22 bp, Artificial Sequence
  Description of Artificial Sequence Synthetic primer
gtcaacctcgcttccccaccct

SEQ ID NO: 22 - DNA, 23 bp, Artificial Sequence
  Description of Artificial Sequence Synthetic primer
cgagtcgtctttctcctgatgat

SEQ ID NO: 23 - DNA, 22 bp, Artificial Sequence
  Description of Artificial Sequence Synthetic primer
ggtgtccatacgcatccttgac

SEQ ID NO: 24 - DNA, 22 bp, Artificial Sequence
  Description of Artificial Sequence Synthetic primer
ctcaaggtgtttgacggcatcc

SEQ ID NO: 25 - DNA, 20 bp, Artificial Sequence
  Description of Artificial Sequence Synthetic primer
ccgagtgatgaccaacaaga

SEQ ID NO: 26 - DNA, 21 bp, Artificial Sequence
  Description of Artificial Sequence Synthetic primer
ggtccttttcaccagcaagct

SEQ ID NO: 27 - DNA, 20 bp, Artificial Sequence
  Description of Artificial Sequence Synthetic primer
atgggcctcttgtagagctt

SEQ ID NO: 28 - DNA, 20 bp, Artificial Sequence
  Description of Artificial Sequence Synthetic primer
agcgctacagcagtgtgaca

SEQ ID NO: 29 - DNA, 20 bp, Artificial Sequence
  Description of Artificial Sequence Synthetic primer
aggactcacactggctcttg

SEQ ID NO: 30 - DNA, 20 bp, Artificial Sequence
  Description of Artificial Sequence Synthetic primer
tactgacatctggtcagaca

SEQ ID NO: 31 - DNA, 21 bp, Artificial Sequence
  Description of Artificial Sequence Synthetic primer
ttgtcatacaccagcttgagt

SEQ ID NO: 32 - DNA, 22 bp, Artificial Sequence
  Description of Artificial Sequence Synthetic primer
ttcaggtccaagtttgcgaagc

SEQ ID NO: 33 - DNA, 20 bp, Artificial Sequence
  Description of Artificial Sequence Synthetic primer
ccaacgctgggcaattcttc

SEQ ID NO: 34 - DNA, 20 bp, Artificial Sequence
  Description of Artificial Sequence Synthetic primer
gcatctgggttctgagcttt

SEQ ID NO: 35 - DNA, 20 bp, Artificial Sequence
  Description of Artificial Sequence Synthetic primer
gtccctcagttctgtccgtg

SEQ ID NO: 36 - DNA, 20 bp, Artificial Sequence
  Description of Artificial Sequence Synthetic primer
atctactgcctggagacctt

SEQ ID NO: 37 - DNA, 18 bp, Artificial Sequence
  Description of Artificial Sequence Synthetic primer
cagcagatgacaggaagg

SEQ ID NO: 38 - DNA, 20 bp, Artificial Sequence
  Description of Artificial Sequence Synthetic primer
atgttggcgagtctcacagg

SEQ ID NO: 39 - DNA, 21 bp, Artificial Sequence
  Description of Artificial Sequence Synthetic primer
gctaggtggcccagtgctact

SEQ ID NO: 40 - DNA, 21 bp, Artificial Sequence
  Description of Artificial Sequence Synthetic primer
atcaaaggacacttcacactc

SEQ ID NO: 41 - DNA, 21 bp, Artificial Sequence
  Description of Artificial Sequence Synthetic primer
tacgttccggaagtaatttgc

SEQ ID NO: 42 - DNA, 23 bp, Artificial Sequence
  Description of Artificial Sequence Synthetic primer
tttgcgggaggcagtgatcttgg

SEQ ID NO: 43 - DNA, 20 bp, Artificial Sequence
  Description of Artificial Sequence Synthetic primer
gcgcggtagttggaggagcg

SEQ ID NO: 44 - DNA, 22 bp, Artificial Sequence
  Description of Artificial Sequence Synthetic primer
tcctgcgaataggcttccggct

SEQ ID NO: 45 - DNA, 21 bp, Artificial Sequence
  Description of Artificial Sequence Synthetic primer
ttctggattccaatgcttcga

SEQ ID NO: 46 - DNA, 22 bp, Artificial Sequence
  Description of Artificial Sequence Synthetic primer
agccgcttgatcttccctggat

SEQ ID NO: 47 - DNA, 22 bp, Artificial Sequence
  Description of Artificial Sequence Synthetic primer
tacttccagccaacctcgtgag

SEQ ID NO: 48 - DNA, 20 bp, Artificial Sequence
  Description of Artificial Sequence Synthetic primer
gaattcaggcccgtagactg

SEQ ID NO: 49 - DNA, 24 bp, Artificial Sequence
  Description of Artificial Sequence Synthetic primer
aaatcccactgcattgccgaagtg

SEQ ID NO: 50 - DNA, 21 bp, Artificial Sequence
  Description of Artificial Sequence Synthetic primer
ctgcaattgagaaacccactg

SEQ ID NO: 51 - DNA, 20 bp, Artificial Sequence
  Description of Artificial Sequence Synthetic primer
tgtacaggaggagaggcatc

SEQ ID NO: 52 - DNA, 22 bp, Artificial Sequence
  Description of Artificial Sequence Synthetic primer
ctcatagcttgaccttcgagtg

SEQ ID NO: 53 - DNA, 20 bp, Artificial Sequence
  Description of Artificial Sequence Synthetic primer
gcaccggattgttcaaagtg

SEQ ID NO: 54 - DNA, 19 bp, Artificial Sequence
  Description of Artificial Sequence Synthetic primer
cattgcccttcctcgtctt

SEQ ID NO: 55 - DNA, 21 bp, Artificial Sequence
  Description of Artificial Sequence Synthetic primer
gcccatcaacactctgaaatg

SEQ ID NO: 56 - DNA, 19 bp, Artificial Sequence
  Description of Artificial Sequence Synthetic primer
tcctttctgagccattgcc

SEQ ID NO: 57 - DNA, 17 bp, Artificial Sequence
  Description of Artificial Sequence Synthetic primer
gccttcacagcgtacga

SEQ ID NO: 58 - DNA, 24 bp, Artificial Sequence
  Description of Artificial Sequence Synthetic primer
agcagcagcacgacagagtaatca

SEQ ID NO: 59 - DNA, 20 bp, Artificial Sequence
  Description of Artificial Sequence Synthetic primer
ttccctcttcactttggctg

SEQ ID NO: 60 - DNA, 21 bp, Artificial Sequence
  Description of Artificial Sequence Synthetic primer
acctgtccaatgtcttcagtc

SEQ ID NO: 61 - DNA, 21 bp, Artificial Sequence
  Description of Artificial Sequence Synthetic primer
ggtggacaaagacaagaggaa

SEQ ID NO: 62 - DNA, 21 bp, Artificial Sequence
  Description of Artificial Sequence Synthetic primer
gtacaaagtcagcatggttcc

SEQ ID NO: 63 - DNA, 21 bp, Artificial Sequence
  Description of Artificial Sequence Synthetic primer
ccagagacagaagaagccaag

SEQ ID NO: 64 - DNA, 21 bp, Artificial Sequence
  Description of Artificial Sequence Synthetic primer
cgccattcagataaaacccag

SEQ ID NO: 65 - DNA, 20 bp, Artificial Sequence
  Description of Artificial Sequence Synthetic primer
tgaagtcgctagagtcccag

SEQ ID NO: 66 - DNA, 17 bp, Artificial Sequence
  Description of Artificial Sequence Synthetic primer
cgaagatggcggaggtg

SEQ ID NO: 67 - DNA, 17 bp, Artificial Sequence
  Description of Artificial Sequence Synthetic probe
acttgccttcattagct

SEQ ID NO: 68 - DNA, 27 bp, Artificial Sequence
  Description of Artificial Sequence Synthetic probe
ttggagaagtacggacattcagactgc

SEQ ID NO: 69 - DNA, 25 bp, Artificial Sequence
  Description of Artificial Sequence Synthetic probe
aacctcagcattgtgatgaccacct

SEQ ID NO: 70 - DNA, 15 bp, Artificial Sequence
  Description of Artificial Sequence Synthetic probe
cttccttcttcatgc

SEQ ID NO: 71 - DNA, 14 bp, Artificial Sequence
  Description of Artificial Sequence Synthetic probe
ggcccagaagcaaa

SEQ ID NO: 72 - DNA, 18 bp, Artificial Sequence
  Description of Artificial Sequence Synthetic probe
gtgtatggaaactgctcc

SEQ ID NO: 73 - DNA, 27 bp, Artificial Sequence
  Description of Artificial Sequence Synthetic probe
acggctccattttctaagattccagca

SEQ ID NO: 74 - DNA, 15 bp, Artificial Sequence
  Description of Artificial Sequence Synthetic probe
gttcgtgtctgacct

SEQ ID NO: 75 - DNA, 16 bp, Artificial Sequence
  Description of Artificial Sequence Synthetic probe
tgtttagccacgatgg